(19) United States
(12) Patent Application Publication
(10) Pub. No.: US 2022/0267329 A1
(43) Pub. Date: Aug. 25, 2022

(54) Dihydro-Spiro[Indoline-3:1'-Isoquinolin]-2-Ones and Their Analogues and Derivatives and Methods of Treating Cancer and Other Diseases

(71) Applicant: The University of Buea, Buea (CM)

(72) Inventors: Simon Mbua Ngale Efange, Buea (CM); Lobe Maloba Mesembe Mabanyi, Buea (CM)

(21) Appl. No.: 17/436,301

(22) PCT Filed: Mar. 5, 2020
(86) PCT No.: PCT/IB2020/051930

§ 371 Date: Sep. 3, 2021

Related U.S. Application Data
(60) Provisional application No. 62/815,660, filed on Mar. 8, 2019.

(51) Int. Cl. C07D 471/10; A61K 31/438; A61P 35/00; A61K 45/06

(52) U.S. Cl. CPC C07D 471/10; A61K 45/06; A61P 35/00; A61K 31/438

(57) ABSTRACT

The present invention is directed to various 3′,4′-dihydro-2′H-spiro[indoline-3:1′-isoquinolin]-2-one compounds and methods for treating disease states and/or conditions which are mediated through sphingosine-1-phosphate receptor(s). The present invention is also directed to the use of these compounds as anticancer agents and as modulators of sphingosine-1-phosphate receptor function in the treatment of disease states and/or conditions which are mediated through these receptors. In addition, the invention relates to pharmaceutical compositions comprising one or more of these compounds alone or in combination with other therapeutic agents. The invention is also directed to methods of treatment of cancer and/or conditions that may respond to the modulation of sphingosine-1-phosphate receptor function and which employ compounds of the present invention or pharmaceutical compositions comprising one or more of the compounds of this invention.

### RELATED APPLICATIONS

This application application is a United States national phase patent application based upon international patent application No. PCT/IB2020/051930 of international filing date Mar. 5, 2020, which claims the benefit of priority of U.S. provisional application Ser. No. 62/815,660 entitled “DIHYDRO-SPIRO[INDOLINE-3:1′-ISOQUINOLIN]-2-ONES AND THEIR ANALOGUES AND DERIVATIVES”, filed 8 Mar. 2019, the entire contents of said application being incorporated by reference herein.

### FIELD OF THE INVENTION

The present invention is directed to various 3′,4′-dihydro-2′H-spiro[indoline-3:1′-isoquinolin]-2-one compounds and methods for synthesis. The present invention is also directed to the use of these compounds as anticancer agents and as modulators of sphingosine-1-phosphate receptor function. In addition, the invention relates to pharmaceutical compositions comprising one or more of these compounds alone or in combination with other therapeutic agents. The invention is also directed to methods of treatment of cancer and/or conditions that may respond to the modulation of sphingosine-1-phosphate receptor function and which employ compounds of the present invention or pharmaceutical compositions comprising one or more of the compounds of this invention.

### BACKGROUND OF THE INVENTION

### Cancer

Cancer refers to a group of more than one hundred diseases characterized by a common feature of rapid and uncontrolled cell proliferation, invasiveness and metastasis, ultimately leading to death. In 2015, worldwide mortality from cancer alone accounted for 8.8 million deaths, thereby positioning cancer as the number two leading cause of death worldwide. According to statistics from the World Health Organization, five forms of cancer account for the most deaths: Lung (1.69 million deaths); Liver (788 000 deaths); Colorectal (774 000 deaths); Stomach (754 000 deaths); and Breast (571 000 deaths). Two other cancers, ovarian and prostate, which affect parts of the male and female reproductive systems are worthy of note. In the US, ovarian cancer accounted for 2.5% of all female cancers but 5% of all cancer deaths due to low survival rates (Cancer Facts & Figures 2018, American Cancer Society). Prostate cancer is the second most common cancer in men, accounting for an estimated 1.1 million cases and 307,000 deaths in 2012 alone [(GLOBOCAN 2012 (IARC), Section of Cancer Surveillance (Nov. 6, 2018)]. In 2010, the total economic cost of cancer was estimated at US 1.16 trillion dollars (WHO Cancer Fact Sheet, 2017). When combined with the human cost of the disease, there is no disputing that cancer constitutes a major public health problem worldwide that requires continued attention.

Cancer arises from the transformation of normal cells to tumor cells through a multistage process that involves a complex interplay of genetic and environmental factors, including carcinogens of three types: physical (e.g., ultraviolet and ionizing radiation); chemical (such as asbestos and components of tobacco smoke); and biological (such as infections from certain viruses or parasites). Consequently, the management of cancer risk and treatment takes many forms. The clinical management of cancer relies on four main pillars: surgery, hormone therapy, radiotherapy and chemotherapy. While these are often used in combination, there is an overwhelming reliance on chemotherapy. However, the effectiveness of many anticancer agents is compromised by chemoresistance. The toxic side effects of some of these drugs also limit their clinical utility. Consequently, there is a continuing need to develop new agents with novel mechanisms of action that can be deployed in the clinic.

### Sphingosine-1-Phosphate (S1P)

Sphingosine-1-phosphate is a lysophospholipid messenger molecule that has pleiotropic effects including cell differentiation, cell migration, cell proliferation, immune response, trafficking of T and B cells, and vascular stability (Gardell et al., 2006; Huwiler and Pfeilschifter, 2008). S1P signaling is partly mediated by a group of five G-protein coupled receptors, S1P1-S1P5. S1P receptors display distinct and sometimes overlapping expression patterns in various tissues and cell types; consequently, these receptors have been assigned a number of cellular functions. S1P receptors have been found to play critical roles in a number of diseases, including cancer, diabetes, inflammation, neurodegeneration, osteoporosis, autoimmune and cardiovascular diseases. As a result, S1P receptors have become interesting therapeutic targets. Modulation of S1P signaling can be achieved through a variety of means, including modulation of S1P production and interference with S1P receptor mediated signaling. Sphingosine-1-phosphate is obtained from the phosphorylation of sphingosine, a reaction catalyzed by the enzymes sphingosine kinase 1 (SphKI) and sphingosine kinase II (SPhKII). Consequently, inhibition of SphKI or SphKII, results in reduced S1P levels. SphKI inhibitors have already shown promise as anticancer agents (Gao et al., 2015). S1P receptor agonists and antagonists also have considerable promise in the treatment of disease, including cancer (Watters et al., 2011). Fingolimod, one of the earliest entries, is a prodrug which is activated by phosphorylation in vivo to yield Fingolimod phosphate (Fingolimod-P). The drug was approved in 2010 for the treatment of multiple sclerosis (reviewed in Chiba & Adachi, 2012); Vermersch, 2018). The compound may also find application in the treatment of ischemia/reperfusion injury, schizophrenia, stroke and neurodegeneration. Moreover, Fingolimod displays anticancer activity in vitro and in vivo, and also potentiates the activity of some anticancer agents. As a result, the drug has been recommended for re-purposing as an anticancer agent (reviewed in Patmanathan et al., 2015). Other S1P receptor modulators have entered the clinic for a variety of applications: psoriasis, ulcerative colitis and inflammatory bowel syndrome (reviewed in Park & Im, 2017). Modulation of sphingosine-1-phosphate receptors is important in breast cancer progression. In this regard, sphingosine 1-phosphate signaling has been found to be critical for the growth and survival of estrogen receptor positive MCF-7 human breast cancer cells (Maiti et al., 2017). Levels of sphingosine 1-phosphate kinase I (SphK1) in triple negative breast cancer (TNBC) patients are significantly higher than levels in patients with other breast tumors. Furthermore, elevated levels of pSphK1 positively correlate with high expression of sphingosine 1-phosphate (S1P), which in turn promotes metastasis of triple negative breast cancer (Wang et al., 2018). While a number of successes have so far been recorded, the majority of compounds appear to belong to only a few chemical classes. In view of the multiple biological actions of S1P, there is little doubt that the role of S1P receptor modulators in the clinic will continue to grow. The development of new chemical entities as S1P receptor modulators may therefore lead to compounds that display unique pharmacological profiles and are better suited to other clinical indications than the current small pool of agents.

### BRIEF DESCRIPTION OF THE INVENTION

This invention is directed to 3′,4′-dihydro-spiro[indoline-3:1′-isoquinolin]-2-one compounds according to the chemical structure I:

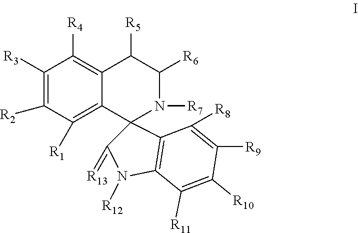

wherein

R₁ is H, OH, C₁-C₆ hydroxyalkyl, halo (F, Cl, Br, I), C₁-C₆ alkoxy (often C₁-C₃ alkoxy, more often OMe), (CH₂)ₙCOOH, (CH₂)ₙC(O)C₀-C₆ alkyl, (CH₂)ₙC(O)OC₁-C₆ alkyl, (CH₂)ₙOC(O)C₀-C₆ alkyl or O(CH₂)ₙaryl,

R₂ and R₃ are each independently H, OH, C₁-C₆ hydroxyalkyl, halo (F, Cl, Br, I), C₁-C₆ alkoxy (often C₁-C₃ alkoxy, more often OMe), (CH₂)ₙCOOH, (CH₂)ₙC(O)C₀-C₆ alkyl, (CH₂)ₙC(O)OC₁-C₆ alkyl, (CH₂)ₙOC(O)C₀-C₆ alkyl, O—(CH₂)ₙaryl, or R₂ and R₃ together form a 5- or 6-membered cycloalkyl or heterocyclic group containing 1, 2 or 3 heteroatoms (O, S, or N), preferably, the heterocyclic group formed is a dioxolanyl (3,4-methylenedioxy), dioxanyl (3,4-ethylenedioxy), dithiolanyl, tetrahydrofuranyl, tetrahydrothiophenyl, pyrrolidinyl, imidazolyl, imidazolinyl, imidazolidinyl, tetrahydropyranyl thianyl, piperidinyl or piperazinyl;

R₄ is H, OH, C₁-C₆ hydroxyalkyl, halo (F, Cl, Br, I), C₁-C₆ alkoxy (often C₁-C₃ alkoxy, more often OMe), (CH₂)ₙCOOH, (CH₂)ₙC(O)C₀-C₆ alkyl, (CH₂)ₙC(O)OC₁-C₆ alkyl, (CH₂)ₙOC(O)C₀-C₆ alkyl, O(CH₂)ₙaryl, (CH₂)ₙAryl (often, phenyl or naphthyl, more often phenyl), (CH₂)ₙHeteroaryl (heteroaryl is preferably pyridyl, thienyl, furyl, pyrrolyl);

R₅ is H, alkyl (preferably C₁-C₆ alkyl), C₁-C₆ alkoxy, (CH₂)ₙAryl (often, phenyl or naphthyl, more often phenyl), (CH₂)ₙHeteroaryl, (aryl is preferably, phenyl, substituted phenyl and heteroaryl is preferably pyridyl, thienyl, furyl, pyrrolyl);

R₆ is H, alkyl (preferably C₁-C₆ alkyl), C₁-C₆ alkoxy, (CH₂)ₙAryl (often, phenyl or naphthyl, more often phenyl), (CH₂)ₙHeteroaryl (often pyridyl, thienyl, furyl, pyrrolyl);

R₇ is alkyl (preferably C₁-C₆ alkyl), (CH₂)ₙAryl (often, phenyl or naphthyl, more often phenyl), (CH₂)ₙC(O)C₀-C₆ alkyl, —(CH₂)ₙR^{N1}N—C(O)—NR^{N2}R^{N3}, (CH₂)ₙ—S(O)₂Aryl, —OC(O)NR^{N1}R^{N2},

R₈ is H, OH, Halo, Nitro, C₁-C₆ hydroxyalkyl, (C₂)ₙNR^{N1}R^{N2}, —(CH₂)ₙ—NR^{N1}—(CH₂)ₙ-Aryl (often, phenyl or naphthyl, more often phenyl), —NR^{N1}SO₂Aryl (often, phenyl or naphthyl, more often phenyl), (CH₂)ₙC₃-C₈cycloalkyl-NR^{N1}R^{N2}, C₁-C₆ alkoxy, O(CH₂)ₙaryl, (C₂)ₙAryl (often, phenyl or naphthyl, more often phenyl), (CH₂)ₙHeteroaryl, C₁-C₆ alkyl, C₂-C₆ vinyl, C₂-C₆ alkynyl, —SO₂NR^{N1}R^{N2}, —OC(O)NR^{N1}R^{N2}, CONR^{N1}R^{N2};

R₉, R₁₀ and R₁₁ are each independently H, OH, Halo, Nitro, C₁-C₆ hydroxyalkyl, (CH₂)ₙNR^{N1}R^{N2}, —(CH₂)ₙ—NR^{N1}—(CH₂)ₙ-Aryl (often, phenyl or naphthyl, more often phenyl), —NR^{N1}SO₂Aryl (often, phenyl or naphthyl, more often phenyl), (CH₂)ₙC₃-C₈cycloalkyl-NR^{N1}R^{N2}, C₁C₆ alkoxy, O(CH₂)ₙaryl, (CH₂)ₙAryl (often, phenyl or naphthyl, more often phenyl), (CH₂)ₙHeteroaryl, C₁-C₆ alkyl, C₂-C₆ vinyl, C₂-C₆ alkynyl, —SO₂NR^{N1}R^{N2}, —OC(O)NR^{N1}R^{N2}, (CH₂)ₙC(O)OC₀-C₆ alkyl or (CH₂)ₙOC(O)C₀-C₆ alkyl, CONR^{N1}R^{N2};

R₁₂ is H, OH, hydroxyalkyl (preferably C₁-C₆ hydroxyalkyl), an optionally substituted (CH₂)ₙAryl (often, phenyl, benzyl or naphthyl, more often benzyl or naphthyl), the Aryl group being optionally substituted with one or two Halo groups, preferably F, Cl or Br, a nitro, CN or a C₁-C₆, preferably a C₁-C₃ alkyl group, preferably R₁₂ is an optionally substituted benzyl group or naphthyl group), (CH₂)ₙC₃-C₈cycloalkyl, (C₂)ₙC(O)NR^{N1} Aryl or (CH₂)ₙ—C(O)C₀-C₆ alkyl;

R₁₃ is O or S;

R^{N1}, R^{N2} and R^{N3} are each independently H or a C₁-C₆ alkyl group which is optionally substituted with one or two hydroxyl groups and up to three halo groups (preferably F);

n is 0-12, preferably 0-6, often 0, 1, 2 or 3, or

a pharmaceutically acceptable salt, stereoisomer, solvate or polymorph thereof.

In embodiments, R₁₂ is a phenyl group, a benzyl group or a naphthyl group, each of which is optionally substituted with a C₁-C₆ alkyl group, a nitro group, a cyano group or one or two halo groups (preferably F, Cl or Br).

In embodiments, the compound is a compound according to the general chemical structures (pharmacores) for compounds 1a-q, 2a-q, 3a-q of FIG. 1A or a compound according to the general chemical structures for compounds 4a-u, 5a-u, and 6a-u of FIG. 1B, wherein R₁ and R₂ are each independently H, halo (preferably F, Cl or Br) or methoxy and R₃ is a phenyl, benzyl or naphthyl group, each of which is optionally substituted with 1 or 2 halo groups (preferably F, Cl or Br), a nitro group, a CN group or a C₁-C₆ alkyl group, preferably a C₁-C₃ group, most often a methyl group.

In embodiments, the compound is one or more compounds set forth in FIGS. 1A and 1B hereof. In embodiments, the compound is a compound or mixture of from 1-3 compounds selected from a group consisting of 2, 3, 4, 5, 6, 7, 8, 9, 10, 11, 12, 13, 14, 15 or more compounds of FIGS. 1A and/or 1B. In embodiments, the compound is a compound or a mixture of 1-3 compounds selected from the group consisting of compounds 1a-i, 1j-q, 2a-i, 2j-q or 3a-q of FIG. 1A. In embodiments, the compound is selected from the group from the group consisting of compounds 4a-u, 5a-u or 6a-u of FIG. 1B. In embodiments the compound is a compound or a mixture of up to 3 compounds selected from the group consisting of compounds 4a-j or 4k-u of FIG. 1B. In embodiments, the compound is a compound or a mixture of up to three compounds selected from the group consisting of compounds 5a-j or 5k-u of FIG. 1B. In embodiments, the compound is a compound or a mixture of up to three compounds selected from the group consisting of compounds 6a-j or 6k-u of FIG. 1B. In embodiments, the compound is a compound or mixture of up to three compounds selected from group consisting of compounds 5a-j or 5k-u or 6a-j or 6k-u. In embodiments, the compound is selected from the group consisting of from 1-7 compounds of the group of compounds 5a-j of FIG. 1B. In embodiments, the compound is selected from the group consisting of compounds 1-7 of compound 5k-u of FIG. 1B. In embodiments, the compound is selected from the group consisting of from 1-7 compounds of the group of compounds 6a-j of FIG. 1B. In embodiments, the compound is selected from the group consisting of compounds 1-7 of compound 6k-u of FIG. 1B. One or more of the compounds of the group 5 compounds (5a-j and 5k-u) is more preferred. Additional preferred compounds include any one or more of compounds 1d, 1f, 1h, 1k, 1l, 1o, 2d, 2l, 2o, 5b and 6d. Further preferred compounds include any one or more of compounds 2f, 2g, 2l, 4e, 5a, 5c, 5a, 5e, 5f and 6b.

In embodiments, the present invention is directed to pharmaceutical compositions comprising an effective amount of a compound as disclosed above, in combination with a pharmaceutically acceptable carrier, additive and/or excipient, optionally in combination with at least one addition bioactive agent, often an additional anticancer agent.

In embodiments, the present invention is directed to a method for modulating (inhibiting, regulating or upregulating as inhibitors, regulators or agonists) sphingosine-1-phosphate receptor function. In embodiments, the modulation of sphingosine-1-phosphate receptor function occurs in a patient or subject, especially a human patient.

In embodiments, the present invention is directed to a method of treating a disease which is mediated through the activity of sphingosine-1-phosphate receptors, the method comprising administering an effective amount of a compound according to the present invention which is a modulator (inhibitor, agonist or regulator) of sphingosine-1-phosphate receptor activity to a patient or subject in need. In embodiments, the present invention is directed to methods of treatment of disease states and/or conditions that may respond to the modulation of sphingosine-1-phosphate receptor function. These disease states and/or conditions include cancer, diabetes, inflammation, neurodegeneration (Alzheimer's disease, Parkinson's disease, Huntington's disease), multiple sclerosis, autoimmune and cardiovascular diseases, including ischemia/reperfusion injury, schizophrenia, stroke, psoriasis, ulcerative colitis and inflammatory bowel syndrome, among others. Additional autoimmune diseases which are treated by the compounds and compositions disclosed herein include for example, rheumatoid arthritis, antiphospholipid antibody syndrome, lupus, chronic urticaria, Sjogren's disease, autoiinmune-related Type 1 diabetes, rheumatoid arthritis (RA), psoriasis/psoriatic arthritis, multiple sclerosis, inflammatory bowel disease (IBD) including Crohn's disease and ulcerative colitis, Addison's disease, Grave's disease, Hashimoto's thyroiditis, Myasthenia gravis, autoimmune vasculitis, pernicious anemia and celiac disease, among others.

In embodiments, the method for synthesizing compounds according to the present invention comprises the combination of a preformed suitably substituted phenethylamine with a suitably substituted 3-oxo-indolin-2-one using any variant of the Pictet Spengler reaction or other cyclodehydrating agents to form the desired spirooxindole. The process involves:
a) Synthesis of a susbstituted isatin, either by i. functionalization through electrophilic aromatic substitution reactions such as halogenation, nitration, sulfonation or other method such as Heck coupling, Suzuki coupling or ii. from a suitably substituted aniline such as p-toluidine, following any of the following methods for the synthesis of isattin and its analogues and derivatives: Sandmeyer, Stolle, Gassman, Martinet, among others.
b) The N-alkylation of the N-unsubstituted isatin by reaction with an alkyl (or arylalkyl) halide preferably in a polar aprotic solvent such as dimethylformamide (DMF) in the presence of a suitable base such as potassium carbonate with heating; and where necessary, the N-acylation of an N-unsubstituted isatin by reaction with an acyl halide preferably in a polar aprotic solvent with a suitable base such as potassium carbonate with minimal heating.
c) Synthesis of a substituted phenethylamine from the aldol condensation of a suitably substituted benzaldehyde with a nitroalkane catalyzed by ammonium acetate, followed by reduction with zinc in hydrochloric acid with heating; and
d) Reaction of the substituted isatin and substituted phenethylamine in polyphosphoric acid at 100° C. to provide the target spirooxindole. In the event where R3 is hydroxyl, the desired spirooxindole is obtained by refluxing the corresponding 3-hydroxyphenethylamine with the substituted isatin in ethanol as previously described by Kametani et al. (1968).

In the case where substitution is needed at the THIQ nitrogen atom (R7 of Structure I), the product obtained from part [d] above may be refluxed in ethanol with the desired precursor such as an alkyl halide or arylalkyl halide in the presence of an organic base (proton scavenger) such as triethylamine. Alternatively, an acyl halide or arylsulfonyl halide may be reacted with the product from [d], in the presence of organic base, such as triethylamine, to yield a carboxamide or sulfonamide, while the reaction of an isocyanate with the said product from [d] can be used to provide the corresponding ureido compound.

### BRIEF DESCRIPTION OF THE FIGURES

FIGS. 1A and 1B show certain preferred compounds of the present invention.

FIG. 2 shows compounds of the prior art.

FIG. 3 illustrates the preparation of compounds of the present invention. Note that step b of the second reaction is the same as step b of the first reaction.

FIG. 4 illustrates the preparation of intermediates used in the synthesis of the compounds of the present invention.

FIG. 5 shows Table 1A shows the percent inhibition of a number of compounds according to the present invention tested against all cancer cell lines in the NCI-60 panel. The specific cell lines affected are indicated in the table. In table 1A, the following abbreviations were used: LEUK: Leukemia; NSCLC: Non-Small Cell Lung Cancer; COL: Colon Cancer; MEL: Melanoma, OVC: Ovarian Cancer; REN: Renal Cancer; PROST: Prostate Cancer; BREAST: Breast Cancer; MDA-MB-468: MDA-MB-468. Note that the asterisk in each of the entries in Table 1A signifies that no antiproliferative activity or antiproliferative activity below thirty percent.

### DETAILED DESCRIPTION OF THE INVENTION

The following terms shall be used throughout the specification to describe the present invention. Where a term is not specifically defined herein, that term shall be understood to be used in a manner consistent with its use by those of ordinary skill in the art.

Where a ranee of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges that may independently be included in the smaller ranges are also encompassed within the invention, subject to any specifically excluded limit in the stated range. Where the stated range includes one or both of the limits, ranges excluding either both of those included limits are also included in the invention. In instances where a substituent is a possibility in one or more Markush groups, it is understood that only those substituents which form stable bonds are to be used.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

It must be noted that as used herein and in the appended claims, the singular forms “a,” “and” and “the” include plural references unless the context clearly dictates otherwise.

Furthermore, the following terms shall have the definitions set out below.

The term “patient” or “subject” is used throughout the specification within context to describe an animal, generally a mammal, especially including a domesticated animal and preferably a human, to whom treatment, including prophylactic treatment (prophylaxis), with the compounds or compositions according to the present invention is provided. For treatment of those infections, conditions or disease states which are specific for a specific animal such as a human patient, the term patient refers to that specific animal. In most instances, the patient or subject of the present invention is a human patient of either or both genders.

The term “effective” is used herein, unless otherwise indicated, to describe an amount of a compound or component which, when used within the context of its use, produces or effects an intended result, whether that result relates to the prophylaxis and/or therapy of an infection and/or disease state within the context of its use or as otherwise described herein. The term effective subsumes all other effective amount or effective concentration terms (including the term “therapeutically effective”) which are otherwise described or used in the present application.

The term “compound” is used herein to describe any specific compound or bioactive agent disclosed herein, including any and all stereoisomers (including diastereomers, individual optical isomers/enantiomers or racemic mixtures and geometric isomers), pharmaceutically acceptable salts and prodrug forms. The term compound herein refers to stable compounds. Within its use in context, the term compound may refer to a single compound or a mixture of compounds as otherwise described herein. It is understood that the choice of substituents or bonds within a Markush or other group of substituents or bonds is provided to form a stable compound from those choices within that Markush or other group.

The term “pharmaceutically acceptable” as used herein means that the compound or composition is suitable for administration to a subject to achieve the treatments described herein, without unduly deleterious side effects in light of the severity of the disease and necessity of the treatment.

“Alkyl” refers to a fully saturated monovalent radical containing carbon and hydrogen, and which may be cyclic, branched or a straight chain. Examples of alkyl groups are methyl, ethyl, n-butyl, n-hexyl, n-heptyl, n-octyl, n-nonyl, n-decyl, isopropyl, 2-methyl-propyl, cyclopropyl, cyclopropylmethyl, cyclobutyl, cyclopentyl, cyclopentylethyl, cyclohexylethyl and cyclohexyl. Preferred alkyl groups are C₀-C₆ alkyl groups (which includes C₀ as H). Even more preferred alkyl groups are C₁-C₆ alkyl groups. “Alkylene” refers to a fully saturated hydrocarbon which is divalent (may be linear, branched or cyclic) and which is optionally substituted. Preferred alkylene groups are C₁-C₆ alkylene groups. Other terms used to indicate substitutuent groups in compounds according to the present invention are as conventionally used in the art.

“Alkylene” refers to a fully saturated hydrocarbon which is divalent (may be linear, branched or cyclic) and which is optionally substituted. Other terms used to indicate substituent groups in compounds according to the present invention are as conventionally used in the art. Thus, the term alkylene aryl includes alkylene phenyl such as a benzyl group or ethylene phenyl group, alkylaryl, includes alkylphenyl such a phenyl group which has alkyl groups as substituents, etc. The bond [symbol] , when used in chemical structures of the present application refers to a single chemical bond, which may be an optional double bond, in context.

The term “aryl” or “aromatic”, in context, refers to a substituted or unsubstituted monovalent aromatic radical having a single ring (e.g., benzene or phenyl) or fused rings (naphthyl). Aromatic heterocycles (which contain 1, 2, 3 or 4 atoms other than carbon (e.g. nitrogen, sulfur, oxytzen, phosphorous or other atoms are heteroaryls in the present application.

The term “heterocycle” or “heterocyclic” shall mean an optionally substituted moiety that is cyclic and contains at least one atom other than a carbon atom, such as a nitrotzen, sulfur, oxygen or other atom. A heterocyclic ring shall contain up to four atoms other than carbon selected from nitrogen, sulfur and oxygen. These rings may be saturated or have unsaturated bonds. As otherwise described, aromatic heterocycles are heteroaryls. Fused rings are also contemplated by the present invention. A heterocycle according to the present invention is an optionally substituted imidazole, a piperazine (including piperazinone), piperidine, furan, pyrrole, imidazole, thiazole, oxazole or isoxazole group, among numerous others. Depending upon its use in context, a heterocyclic ring may be saturated and/or unsaturated.

“Alkoxy” as used herein refers to an alkyl group bound through an ether linkage; that is, an “alkoxy” group may be represented as —O -alkyl where alkyl is as defined above.

“Hydrocarbon” or “hydrocarbyl” refers to any radical containing carbon and hydrogen, which may be straight, branch-chained or cyclic in nature. Hydrocarbons include linear, branched and cyclic hydrocarbons, including alkyl groups, alkylene groups and unsaturated hydrocarbon groups, which may be optionally substituted. Hydrocarbyl groups may be fully saturated or unsaturated, containing one or more double (“ene”) or triple (“yne”) bonds.

The term “bioactive agent” refers to any biologically active compound or drug which may be formulated for use in the present invention. Exemplary bioactive agents include the compounds according to the present invention which are used to treat cancer as well as other disease states and/or conditions which are otherwise described herein.

The terms “treat”, “treating”, and “treatment”, are used synonymously to refer to any action providing a benefit to a patient at risk for or afflicted with a disease, including improvement in the condition through lessening or suppression of at least one symptom, delay in progression of the disease or delay in the onset of the disease, etc. Treatment, as used herein, encompasses prophylactic and therapeutic treatment, depending on the context of the treatment used. Compounds according to the present invention can, for example, be administered prophylactically to a mammal in advance of the occurrence of disease to reduce the likelihood of that disease. Prophylactic administration is effective to reduce or decrease the likelihood of the subsequent occurrence of disease in the mammal or decrease the severity of disease that subsequently occurs. Alternatively, compounds according to the present invention can, for example, be administered therapeutically to a mammal that is already afflicted by disease. In one embodiment of therapeutic administration, administration of the present compounds is effective to eliminate the disease and produce a remission or substantially eliminate the symptoms of a disease state and/or condition; in another embodiment, administration of the compounds according to the present invention is effective to decrease the severity of the disease or lengthen the lifespan of the mammal so afflicted, in the case of cancer, as well as other diseases and conditions that are mediated through sphingosine-1-phosphate receptor activity in a patient or subject in need. In embodiments, the present invention is directed to methods of treatment of disease states and/or conditions that may respond to the modulation of sphingosine-1-phosphate receptor function, which disease states and/or conditions include cancer, diabetes, inflammation, neurodegeneration (Alzheimer's disease, Parkinson's disease, Huntington's disease), multiple sclerosis, autoimmune and cardiovascular diseases, including ischemia/reperfusion injury, schizophrenia, stroke, psoriasis, ulcerative colitis and inflammatory bowel syndrome, among others.

The term “pharmaceutically acceptable” as used herein means that the compound or composition is suitable for administration to a subject to achieve the treatments described herein, without unduly deleterious side effects in light of the severity of the disease and necessity of the treatment.

The term “inhibit” as used herein refers to the partial or complete elimination of a potential effect such as a symptom or a secondary condition of a disease state, while inhibitors are compounds that have the ability to inhibit.

The term “prevention” when used in context shall mean “reducing the likelihood” or preventing a condition or disease state from occurring as a consequence of administration or concurrent administration of one or more compounds or compositions according to the present invention, alone or in combination with another agent. It is noted that prophylaxis will rarely be 100% effective; consequently the terms prevention and reducing the likelihood are used to denote the fact that within a given population of patients of subjects, administration with compounds according to the present invention will reduce the likelihood or inhibit a particular condition or disease state (in particular, the worsening of a disease state such as the growth and/or metastasis of cancer or other accepted indicators of disease progression (e.g., in the case of inflammatory and neurologic diseases) from occurring.

The term “cancer” shall refer to a proliferation of tumor cells having the unique trait of loss of normal controls, resulting in unregulated growth, lack of differentiation, local tissue invasion, and/or metastasis. As used herein, neoplasms include, without limitation, morphological irregularities in cells in tissue of a subject or host, as well as pathologic proliferation of cells in tissue of a subject, as compared with normal proliferation in the same type of tissue. Additionally, neoplasms include benign tumors and malignant tumors (e.g., colon tumors) that are either invasive or noninvasive. Malignant neoplasms are distinguished from benign neoplasms in that the former show a greater degree of anaplasia, or loss of differentiation and orientation of cells, and have the properties of invasion and metastasis. The term cancer also within context, includes drug resistant cancers, including multiple drug resistant cancers, tnetastatic cancers and recurrent cancers. Examples of neoplasms or neoplasias from which the target cell of the present invention may be derived include, without limitation, carcinomas (e.g., squamous-cell carcinomas, adenocarcinomas, hepatocellular carcinomas, and renal cell carcinomas), particularly those of the bladder, bone, bowel, breast, cervix, colon (colorectal), esophagus, head, kidney, liver, lung, nasopharyngeal, neck, ovary, pancreas, prostate, and stomach; leukemias, such as acute myelogenous leukemia, acute lymphocytic leukemia, acute promyelocytic leukemia (APL), acute T-cell lymphoblastic leukemia, adult T-cell leukemia, basophilic leukemia, eosinophilic leukemia, granulocytic leukemia, hairy cell leukemia, leukopenic leukemia, lymphatic leukemia, lymphoblastic leukemia, lymphocytic leukemia, megakaryocytic leukemia, micromyeloblastic leukemia, monocytic leukemia, neutrophilic leukemia and stem cell leukemia; benign and malignant lymphomas, particularly Burkitt's lymphoma, Non-Hodgkin's lymphoma and B-cell lymphoma; benign and malignant melanomas; myeloproliferative diseases; sarcomas, particularly Ewing's sarcoma, hemangiosarcoma, Kaposi's sarcoma, liposarcoma, myosarcomas, peripheral neuroepithelioma, and synovial sarcoma; tumors of the central nervous system (e.g., gliomas, astrocytomas, oligodendrogliomas, ependymomas, gliobastomas, neuroblastomas, ganglioneuromas, ganuliogliomas, medulloblastomas, pineal cell tumors, meningiomas, meningeal sarcomas, neurofibromas, and Schwannomas); germ-line tumors (e.g., bowel cancer, breast cancer, prostate cancer, cervical cancer, uterine cancer, lung cancer (e.g., small cell lung cancer, mixed small cell and non-small cell cancer, pleural mesothelioma, including metastatic pleural mesothelioma small cell lung cancer and non-small cell lung cancer), ovarian cancer, testicular cancer, thyroid cancer, astrocytoma, esophageal cancer, pancreatic cancer, stomach cancer, liver cancer, colon cancer, and melanoma); mixed types of neoplasias, particularly carcinosarcoma and Hodgkin's disease; and tumors of mixed origin, such as Wilms' tumor and teratocarcinomas, among others.

In addition to the treatment of ectopic cancers as described above, the present invention also may be used preferably to treat ectopic cancers such as choriocarcinoma, testicular choriocarcinoma, non-seminomatous germ cell testicular cancer, placental cancer (trophoblastic tumor) and embryonal cancer, among others.

The term “neoplasia” refers to the uncontrolled and progressive multiplication of tumor cells, under conditions that would not elicit, or would cause cessation of, multiplication of normal cells. Neoplasia results in a “neoplasm”, which is defined herein to mean any new and abnormal growth, particularly a new growth of tissue, in which the growth of cells is uncontrolled and progressive. Thus, neoplasia includes “cancer”, which herein refers to a proliferation of tumor cells having the unique trait of loss of normal controls, resulting in unregulated growth, lack of differentiation, local tissue invasion, and/or metastasis or recurrence of cancer.

As used herein, neoplasms include, without limitation, morphological irregularities in cells in tissue of a subject or host, as well as pathologic proliferation of cells in tissue of a subject, as compared with normal proliferation in the same type of tissue. Additionally, neoplasms include benign tumors and malignant tumors (e.g., colon tumors) that are either invasive or noninvasive. Malignant neoplasms are distinguished from benign neoplasms in that the former show a greater degree of anaplasia, or loss of differentiation and orientation of cells, and have the properties of invasion and metastasis. Examples of (many of which are identified above) include neoplasms or neoplasias from which the target cell of the present invention may be derived include, without limitation, carcinomas (e.g., squamous-cell carcinomas, basal cell carcinomas, adenocarcinomas, hepatocellular carcinomas, and renal cell carcinomas), particularly those of the bladder, bowel, breast, cervix, colon, esophagus, head, kidney, liver, lung, neck, ovary, pancreas, prostate, and stomach; leukemias; benign and malignant lymphomas, particularly Burkitt's lymphoma and Non-Hodgkin's lymphoma; benign and malignant melanomas; myeloproliferative diseases; sarcomas, particularly Ewing's sarcoma, hemangiosarcoma, Kaposi's sarcoma, liposarcoma, myosarcomas, peripheral neuroepithelioma, and synovial sarcoma; tumors of the central nervous system (e.g., gliomas, astrocytomas, oligodendrogliomas, ependymomas, glioblastomas, neuroblastomas, ganglioneuromas, gangliogliomas, medulloblastomas, pineal cell tumors, meningiomas, meningeal sarcomas, neurofibromas, and Schwannomas); germ-line tumors (e.g., bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, and melanoma); mixed types of neoplasias, particularly carcinosarcoma and Hodgkin's disease; and tumors of mixed origin, such as Wilms' tumor and teratocarcinomas (Beers and Berkow (eds.), The Merck Manual of Diagnosis and Therapy, 17.sup.th ed. (Whitehouse Station, N.J.: Merck Research Laboratories, 1999) 973-74, 976, 986, 988, 991. Cancers which may be treated pursuant to the present invention include metastatic cancers and recurrent cancers.

The term “additional anti-cancer agent” is used to describe an additional compound which may be coadministered with one or more compounds of the present invention in the treatment of cancer. Such agents include, for example, everolimus, trabectedin, abraxane, TLK 286, AV-299, DN-101, pazopanib, GSK690698, RTA 744, ON 0910.Na, AZD 6244 (ARRY-142886), AMN-107, TK1-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulator, a Bcl-2 inhibitor, an HDAC inhibitor, a c-MET inhibitor, a PARP inhibitor, a Cdk inhibitor, an EGFR TK inhibitor, an IGFR-TK inhibitor, an anti-HGF antibody, a PI3 kinase inhibitors, an AKT inhibitor, a JAK/STAT inhibitor, a checkpoint-1 or 2 inhibitor, a focal adhesion kinase inhibitor, a Map kinase kinase (mek) inhibitor, a VEGF trap antibody, pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-1-TM-601, ALT-110, BIO 140, CC 8490, cilengitide, gimatecan, IL13-PE38QQR, INO 1001, IPdR₁ KRX-0402, lucanthone, LY 317615, neuradiab, vitespan, Rta 744, Sdx 102, talampanel, atrasentan, Xr 311, romidepsin, ADS-100308, sunitinib, 5-fluorouracil, vorinostat, etoposide, gemcitabine, doxorubicin, irinotecan, liposomal doxorubicin, 5′-deoxy-5-fluorouridine, vincristine, temozolomide, ZK-304709, seliciclib, PD0325901, AZD-6244, capecitabine, L-Glutamic acid, N-[4-[2-(2-amino-4,7-dihydro-4-oxo-1 H-pyrrolo[2,3-d]pyrimidin-5-yl)ethyl]benzoyl]-, disodium salt salt, heptahydrate, camptothecin, PEG-labeled irinotecan, tamoxifen, toremifene citrate, anastrazole, exemestane, letrozole, DES(diethylstilbestrol), estradiol, estrogen, conjugated estrogen, bevacizumab, IMC-1C11, CHIR-258,); 3-[5-(methylsulfonylpiperadinemethyl)-indolylj-quinolone, vatalanib, AG-013736, AVE-0005, the acetate salt of [D-Ser(Bu t) 6, Azgly 10] (pyro-Glu-His-Trp-Ser-Tyr-D-Ser(Bu t)-Leu-Arg-Pro-Azgly-NH₂ acetate [C₅₉H₈₄N₁₈Oi₄-(C₂O₄O₂)ₓ where x=1 to 2.4], goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, Ionafamib, BMS-214662, tipifarnib; amifostine, NVP-LAQ824, suberoyl analide hydroxamic acid, valproic acid, trichostatin A, FK-228, SU11248, sorafenib, KRN-951, aminoglutethimide, amsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gemcitabine, gleevac, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, irinotecan, topotecan, doxorubicin, docetaxel, vinorelbine, bevacizutnab (monoclonal antibody) and erbitux, cremophor-free paclitaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, PTK787/ZK 222584, VX-745, PD 184352, rapamycin, 40-O-(2-hydroxyethyl)-rapamycin, temsirolinius, AP-23573, RAD001, ABT-578, BC-210, LY294002, LY292696, LY293684, LY293646, wortmannin, ZM336372, L-779,450, PEG-filarastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, genduzumab, hydrocortisone, interleukin-11, dexrazoxane, alemtuzumab, all-transretinoic acid, ketoconazole, interleukin-2, megestrol, immune globulin, nitrogen mustard, methylprednisolone, ibrituumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa, darbepoetin alfa, ipilumumab, vemurafenib among others among others, including immunotherapy agents such as IDO inhibitors (an inhibitor of indoleamine 2,3-dioxygenase (IDO) pathway) such as Indoximod (NLG-8187), Navoximod (GDC-0919) and NLG802, PDL1 inhibitors (an inhibitor of programmed death-ligand 1) including, for example, nivolumab, durvalumab and atezolizumab, PD1 inhibitors such as pembrolizumab (Merck) and CTLA-4 inhibitors (an inhibitor of cytotoxic T-lymphocyte associated protein 4/cluster of differentiation 152), including ipilimumab and tremelimumab, among others.

The term “co-administration” or “adjunct therapy” shall mean that at least two compounds or compositions are administered to the patient at the same time, such that effective amounts or concentrations of each of the two or more compounds may be found in the patient at a given point in time. Although compounds according to the present invention may be co-administered to a patient at the same time, the term embraces both administration of two or more agents at the same time or at different times, including sequential administration. Preferably, effective concentrations of all co-administered compounds or compositions are found in the subject at a given time. The term co-administration or adjunct therapy also contemplates other bioactive agents being coadministered with pharmaceutical compositions according to the present invention, especially where a cancer has metastasized or is at risk for metastasis.

### The Following Abbreviations apply to the Description of the Present Invention, especially the Examples

EtOAc means Ethyl acetate.
PPA means Polyphosphoric acid.
NSCLC means NonSmall Cell Lung Cancer
CNS means Central Nervous System
S1P means Sphingosine 1-Phosphate

The present invention includes the compositions comprising the pharmaceutically acceptable salt. i.e., the acid or base addition salts of compounds of the present invention and their derivatives. The acids which may be used to prepare the pharmaceutically acceptable acid addition salts of the aforementioned base compounds useful in this invention are those which form non-toxic acid addition salts, i.e., salts containing pharmacologically acceptable anions, such as the hydrochloride, hydrobromide, hydroiodide, nitrate, sulfate, bisulfate, phosphate, acid phosphate, acetate, lactate, citrate, acid citrate, tartrate, bitartrate, succinate, maleate, fumarate, gluconate, saccharate, benzoate, methanesulfonate, ethanesulfonate, benzenesulfonate, p-toluenesulfonate and pamoate [i.e., 1,1′-methylene-bis-(2-hydroxy-3 naphthoate)]salts, among others.

Pharmaceutically acceptable base addition salts may also be used to produce pharmaceutically acceptable salt forms of the compounds according to the present invention. The chemical bases that may be used as reagents to prepare pharmaceutically acceptable base salts of the present compounds that are acidic in nature are those that form non-toxic base salts with such compounds. Such non-toxic base salts include, but are not limited to those derived from such pharmacologically acceptable cations such as alkali metal cations (eg., potassium and sodium) and alkaline earth metal cations (e, calcium and magnesium), ammonium or water-soluble amine addition salts such as N-methylglucamine-(meglumine), and the lower alkanolammonium and other base salts of pharmaceutically acceptable organic amines, among others.

Compounds according to the present invention may be readily formulated into pharmaceutical compositions, useful in the treatment of disease states and/or conditions as otherwise described herein. These disease states and/or conditions include cancer, diabetes, inflammation, neurodegeneration (Alzheimer's disease, Parkinson's disease, Huntington's disease), autoimmune and cardiovascular diseases, including ischemia/reperfusion injury, schizophrenia, stroke, psoriasis, ulcerative colitis and inflammatory bowel syndrome, among others.

Pharmaceutical compositions comprise an effective amount of one or more compounds according to the present invention in combination with a pharmaceutically acceptable carrier, additive or excipient, optionally in combination with at least one additional anticancer agent.

As noted above, the compounds and method of the invention modulate sphingosine-1-phosphate receptor function as otherwise described herein, and are useful for the inhibition (including prophylaxis) and/or treatment of cancer, diabetes, inflammation, neurodegeneration (Alzheimer's disease, Parkinson's disease, Huntington's disease), autoimmune and cardiovascular diseases, including ischemia/reperfusion injury, schizophrenia, stroke, psoriasis, ulcerative colitis and inflammatory bowel syndrome, among others.

In methods according to the present invention, subjects or patients in need are treated with the present compounds, pharmaceutical compositions in order to inhibit, reduce the likelihood or treat a disease state, condition and/or infection as otherwise described herein. The disease states, conditions and infections treated by the present compounds and compositions are readily recognized and diagnosed by those of ordinary skill in the art and treated by administering to the patient an effective amount of one or more compounds according to the present invention.

Regardless of the mechanism, the compounds of the present invention may be used to treat disease states or conditions in patients or subjects who suffer from those conditions or disease states or are at risk for those conditions. In this method a compound in an effective amount is administered to a patient in need of therapy to treat the condition(s) or disease state(s). These disease states and conditions include obesity and diabetes and related disease states and conditions which occur associated with these conditions, such as insulin resistance, metabolic syndrome and the like.

Generally, dosages and routes of administration of the compound are determined according to the size and condition of the subject, according to standard pharmaceutical practices. Dose levels employed can vary widely, and can readily be determined by those of skill in the art. Typically, amounts in the milligram up to gram quantities are employed. The composition may be administered to a subject by various routes, e.g. orally, transdermally, perineurally or parenterally, that is, by intravenous, subcutaneous, intraperitoneal, or intramuscular injection, among others, including buccal, rectal, and transdermal administration. Subjects contemplated for treatment according to the method of the invention include humans, companion animals, laboratory animals, and the like.

Formulations containing the compounds according to the present invention may take the form of solid, semi-solid, lyophilized powder, or liquid dosage forms, such as, for example, tablets, capsules, powders, sustained-release formulations, solutions, suspensions, emulsions, suppositories, creams, ointments, lotions, aerosols, patches or the like, preferably in unit dosage forms suitable for simple administration of precise dosages.

Pharmaceutical compositions according to the present invention typically include a conventional pharmaceutical carrier or excipient and may additionally include other medicinal agents, carriers, adjuvants, additives and the like. Preferably, the composition is about 0.1% to about 85%, about 0.5% to about 75% by weight of a compound or compounds of the invention, with the remainder consisting essentially of suitable pharmaceutical excipients. For oral administration, such excipients include pharmaceutical grades of mannitol, lactose, starch, magnesium stearate, sodium saccharine, talcum, cellulose, glucose, gelatin, sucrose, magnesium carbonate, and the like. If desired, the composition may also contain minor amounts of non-toxic auxiliary substances such as wetting agents, emulsifying agents, or buffers.

Liquid compositions can be prepared by dissolving or dispersing the compounds (about 0.5% to about 20% by weight or more), and optional pharmaceutical adjuvants, in a carrier, such as, for example, aqueous saline, aqueous dextrose, glycerol, or ethanol, to form a solution or suspension. For use in oral liquid preparation, the composition may be prepared as a solution, suspension, emulsion, or syrup, being supplied either in liquid form or a dried form suitable for hydration in water or normal saline.

When the composition is employed in the form of solid preparations for oral administration, the preparations may be tablets, granules, powders, capsules or the like. In a tablet formulation, the composition is typically formulated with additives, e.g. an excipient such as a saccharide or cellulose preparation, a binder such as starch paste or methyl cellulose, a filler, a disintegrator, and other additives typically used in the manufacture of medical preparations.

An injectable composition for parenteral administration will typically contain the compound in a suitable i.v. solution, such as sterile physiological salt solution. The composition may also be formulated as a suspension in a lipid or phospholipid, in a liposomal suspension, or in an aqueous emulsion.

Methods for preparing such dosage forms are known or are apparent to those skilled in the art; for example, see Remington's Pharmaceutical Sciences (17th Ed., Mack Pub. Co., 1985). The composition to be administered will contain a quantity of the selected compound in a pharmaceutically effective amount for modulating GTPase in a subject according to the present invention in a subject.

### EXAMPLES

### Drug Discovery by Combination of Privileged Scaffolds: Spiroheterocycles

The search for novel bioactive molecules has employed many strategies including, random screening, ethnomedically-directed screening, construction of focused compound libraries, the construction of diversity driven libraries, and in silico screening. At the level of molecular optimization, several methods have been adopted such as, the incorporation new pharmacophores into existing molecular scaffolds through the insertion of new functional groups, and the construction of hybrid molecules that combine the scaffolds of two bioactive or privileged scaffolds to form a hybrid scaffold. The latter technique, termed molecular hybridization (Lazar et al., 2004; Viegas-Junior et al, 2007), is the approach used in this invention.

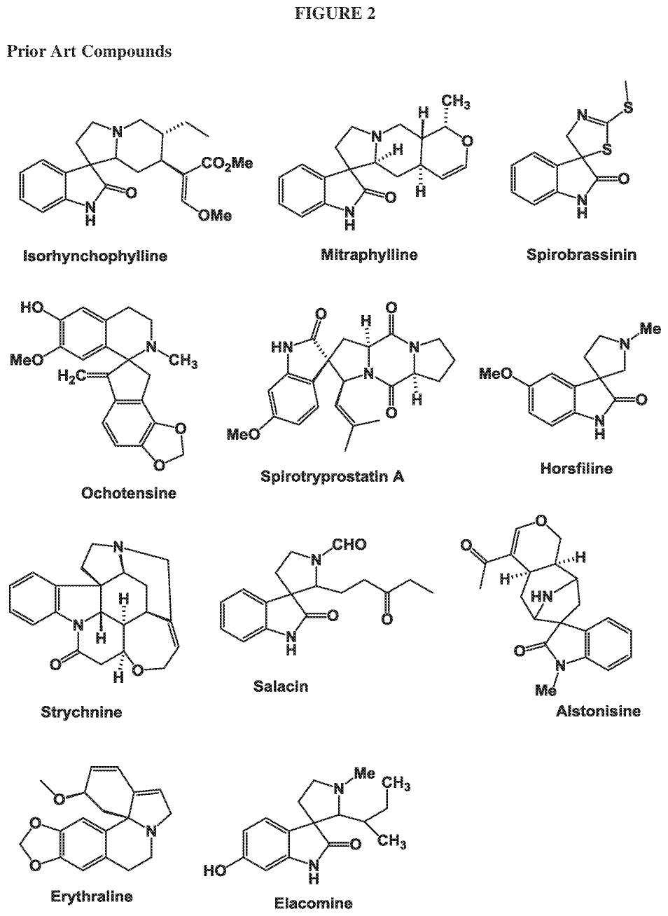

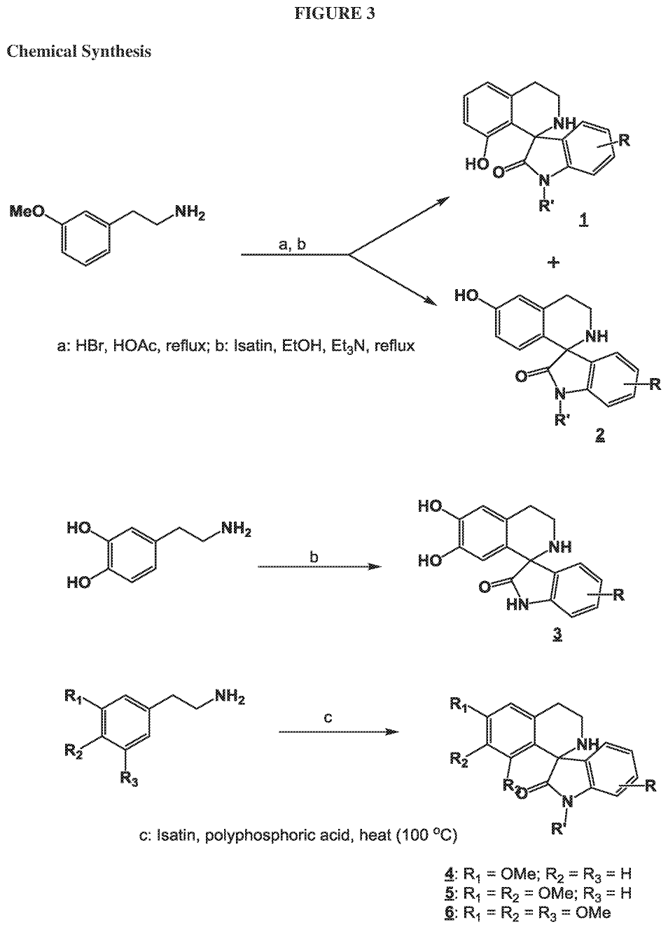

Privileged scaffolds are molecular frameworks that occur frequently in biologically active molecules and are therefore thought to be recognized by many biological targets (Evans et al., 1988; reviewed in Welsch et al., 2010; Zhao & Dietrich, 2015). The combination of two privileged scaffolds can therefore be expected to yield a new scaffold that may serve as a multiple-target drug, interacting with some or all of the biological targets of the constituent fragments, or display pharmacological properties that are separate and distinct from those of the building blocks. Consequently, molecular hybridization has become a favorite tool of medicinal chemists (reviewed in Pawelczyk et al., 2018). The combination of two or more molecular scaffolds can be accomplished either indirectly through the use of linkers or directly by way of single or multiple bonds. In the latter strategy, the two scaffolds can be fused linearly through two atoms or spirally through a single atom. In the case where the two fragments share a single atom, the planes of contributing (cyclic) scaffolds intersect orthogonally. The resulting corkscrew arrangement endows the molecule with a level of three-dimensionality that exceeds that of the planar molecules. These spirocycles are thus presumed to more efficiently project functionalities in three-dimensional space and thereby explore the cognate biological targets more effectively that the planar molecules. Owing to their distinct structural features, spirocycles have attracted great interest as scaffolds for drug discovery in recent years (Zheng & Tice, 2016). The high level of interest in this class of molecules is justified by their occurrence in nature and their diverse pharmacological properties which include anticancer, antimalarial, anticonvulsant, antimicrobial, antimycobacterial, antiviral, and central nervous system activities. Notable examples of naturally occurring spirofused compounds include rhynchophylline (Uncaria tomentosa), mitraphylline (Hallea stipulosa), horsfiline (Horsfieldia superba), strychnine (Strychnos sp), erythraline (Erythrina sp), ibolutine (Voacanga africana), onchotensine (Corydalis ochatensis), ecteinascidin(Ecteinascidia turbinata), spirotryprostatin A (Aspergillus fumigatus), strychnophylline (Strychnos sp), spiro-brassinin (Brassica campestris), dl-salacin (Uncariasalaccensis), elacomine (Elaeagnus commutala), alstonisine (Alstonia Muelleriana) and neosurugatoxin (Babylonia japonica) (FIG. 2). Predictably, synthetic spirocycles similarly display a wide range of pharmacological properties (reviewed in Zheng & Tice, 2016; Ziarani et al., 2016).

Naturally occurring heterocyclic spirocycles or spiroheterocyles fall into a number of structural classes. A large number of these spirofused compounds are found to contain either the 2-oxotryptamine or phenethylamine motifs. The recurrence of these molecular fragments can be attributed to the frequent use of tryptophan (or tryptamine), tyrosine (or dopamine) as starting materials in the biosynthesis of these secondary metabolites, and the ease with which these biogenic arylethylamines undergo cyclization through the intramolecular Mannich and Pictet-Spengler-type reactions under physiological conditions. Tryptamine, its oxidized form 2-oxotryptamine and hydroxyphenethylamines like dopamine are therefore uniquely suited to the facile formation of spirofused compounds. For tryptamine, spirofusion can occur by the Pictet-Spengler reaction with a ketone to yield a 1,1-spirofused tetrahydro-β-carboline (THβC). On the other hand, 2-oxotryptamine, reacts with carbonyl containing compounds through the intramolecular Mannich reaction to yield 3,3-spirofused compounds such as rhynchophylline which are called spirooxindoles. Many of such spirocycles are found in nature. Similar to tryptamine, the phenethylamines react by Pictet-Spengler reaction to yield 1,1-spirofused 1,2,3,4-tetrahydroisoquinolines (THIQs) such as erythraline. The Pictet-Spengler and Mannich reactions can therefore be used to produce, with relative ease, large numbers of new chemotypes (or privileged scaffold hybrids) that couple THIQ (or THβC) with other carbonyl-containing privileged scaffolds through spirofusion. One such carbonyl-containing scaffold is isatin, an endogenous compound obtained from the fruits of Couroupita guianensis and also found in the secretions from the parotid gland of the Bufo frog (reviewed in Khan & Maalik, 2015). The Pictet-Spengler reaction of tryptamine (or analogues thereof) with isatin yields 2′,3′,4′,9′-tetrahydrospiro[indoline-3,1′-pyrido[3,4-b]indol]-2-ones, a class of synthetic spirooxindoles. Formally, these compounds may be regarded as resulting from the hybridization of tetrahydro-β-carboline (THBC) and oxindole. They differ from many of the naturally occurring spirooxindoles, such as rhynchophylline, in that the oxindole fragment is located outside the tryptamine motif This new class of spirooxindoles has generated tremendous interest in drug discovery and provided some interesting compounds.

Although the literature abounds with reports of the isolation, synthesis and pharmacological properties of naturally occurring and synthetic spirooxindoles that are based on the tryptamine or 2-oxotryptamine backbone, there is little information on synthetic spirooxindoles that are based on the phenethylamine scaffold, specifically dihydro-spiro[indoline-3:1′-isoquinolin]-2-one (Structure I), and compounds derived from this scaffold. This molecular framework was first described in 1972 as an unexpected product from the attempted synthesis of 1-benzyl-1,2,3,4-tetrahydroisoquines (Brouwer et al., 1972). Since then, this scaffold has received little attention in drug discovery or other applications.

The dihydro-spiro[indoline-3:1′-isoquinolin]-2-one scaffold combines two well-known privileged scaffolds, oxindole and THIQ. Therefore, this hybrid scaffold may reasonably be expected to produce compounds that display interesting pharmacological profiles including anticancer activity, antiplasmodial activity. A brief review of the pharmacological profiles of THIQs, isatins(3-keto-oxindoles) and oxindoles is presented below.

1,2,3,4-Tetrahydroisoquinolines (THIQ): Naturally occurring and synthetic THIQs display a wide range of biological activities including CNS, cardiovascular, antitumor, antibacterial, antimicrobial, antitubercular, antiviral, antifungal, antileishmanial, antitrypanosomal and antiplasmodial activities (reviewed in Ngo Hanna et al., 2014). These compounds include simple THIQs, naphthylisoquinolines, benzylisoquinolines and bisbenzylisoquinolines and spirofused THIQs. The marine-derived THIQ Trabectidin, is active against prostate cancer and many other tumors. The compound induces apoptosis and increases in caspase-3, -8, -9 and p53 (Acikgoz et al. 2015). In acute myeloid leukemia, berberine is found to downregulate CDK2 and upregulate the tumor suppressor genes p21, p27 and p53 (Mohammadi et al., 2017). Furthermore, apomorphine potently inhibits the MDM2-p53 interaction (Ishiba, 2017). THIQs also reverse multidrug resistance in cancer (Zinzi et al., 2014); inhibit histone deacetylase, an important therapeutic target in cancer (Chen et al., 2014); exhibit antiproliferative active against glioblastoma (Mohler et al., 2006); induce G2/M cell cycle arrest and apoptosis arrest mitosis through the disruption of microtubule dynamics (DeBono et al., 2015); and display antiproliferative against breast cancer cells (Gangaparum et al, 2014). In the continuing search for anticancer agents with novel mechanisms of action, Qing et al. (2017) have reviewed the activity of 379 compounds belonging to 26 classes of isoquinolines. Their survey identified some aporphine, oxoaporphine, isooxoaporphine, bisbenzylisoquinoline, and protoberberine alkaloids as attractive target molecules for anticancer drug discovery (Qing et al., 2017). Finally, the Erythrina alkaloids erythraline, erysodine and erysotrine display modest anticancer activity against Hep-G2 and HEP cell lines (Mohammed et al., 2011). Taken together, the foregoing clearly demonstrates the recurrence of the THIQ scaffold in molecules that display anticancer activity.
Isatin and 3-deoxo-isatin (Oxindole): Isatin is a naturally occurring compound isolated from the fruits of Couroupita guianensis and also found in the secretions from the parotid gland of the Bufo frog. Isatin derivatives, including oxindole, have also been found as secondary, metabolites in marine molluscs, fungi, symbiotic bacteria, plants and urine (reviewed in Vine et al. 2013; Khan & Maalik, 2015)). Originally discovered as a product of the degradation of isatin and its analogues and derivatives demonstrate a broad range of biological properties. Isatin derivatives display antimalarial, antifungal, anticonvulsant, anti-HIV and antibacterial activities (reviewed in Khan & Maalik, 2015; Nesi et al., 2013; Kumar et al., 2013). The 3-deoxo-3-substituted isatins (3-substituted oxindoles) also display a range of activities that largely parallels that of the isatins. The 3-hydroxy-3-arylalkyloxindoles are radical scavengers; these compounds also display antifungal and antibacterial activity (Dandia et al., 2014). The corresponding 3-hydroxy-3-aryloxindoles as display anticancer activity (reviewed in Welsch et al., 2010). As a result, both isatin and oxindole are recognized as privileged scaffolds in anticancer drug discovery. N-(substituted) benzylisatins have been reported to exhibit anticancer activity (Modi et al., 2010; Vine et al., 2013) possibly through the inhibition of caspase (Chu et al., 2005, 2007) and p53 induction (Davidovich et al., 2015). In these studies, it was shown that inhibition of caspase activity was sensitive to substitution on the benzyl fragment and on the isatin backbone itself. The 3-arylidene oxindoles and isatinylchalcones display anticancer activity and 3-arylidenes oxindoles such as sunitinib have since entered the clinic as a treatment for various cancers. The 3-arylidene oxindoles have also been shown to inhibit the tumor suppressor gene p53 (Zheng et al., 2014). Therefore, isatin and oxindole are clearly established as privileged scaffolds for anticancer activity.

Isatin, the precursor used here, is an oxidized indole found in mammalian brain and peripheral tissues (reviewed in a Medvedev et al., 2007). On the other hand, THIQs arise naturally from the Pictet-Spengler type condensation of biogenic amines and aldehydes/ketones; therefore, the hybridization of THIQ and oxindole which occurs by Pictet-Spengler reaction at the C1 position of THIQ may be regarded as the biomimetic combination of compounds arising from two endogenous products. The proposed molecular hybrids may therefore be expected to recognize important biomolecular targets and thus display potentially useful pharmacological activities.

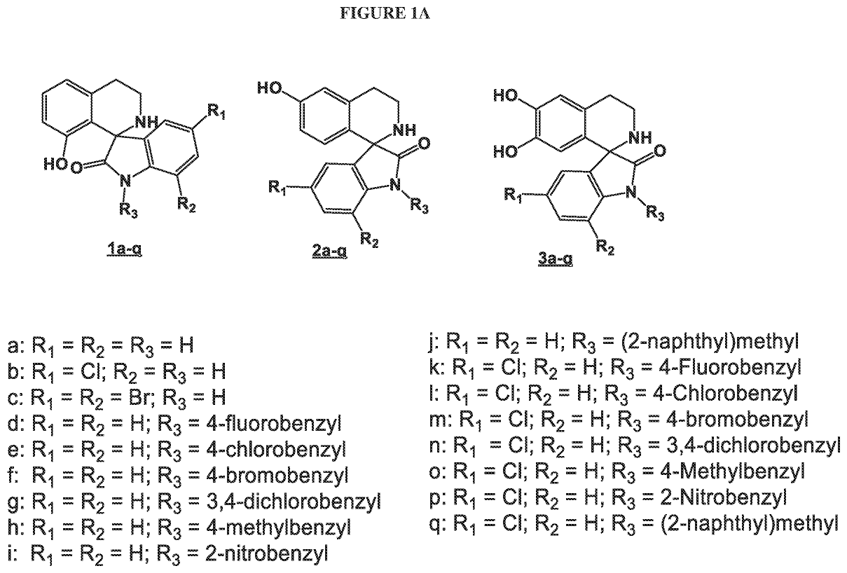

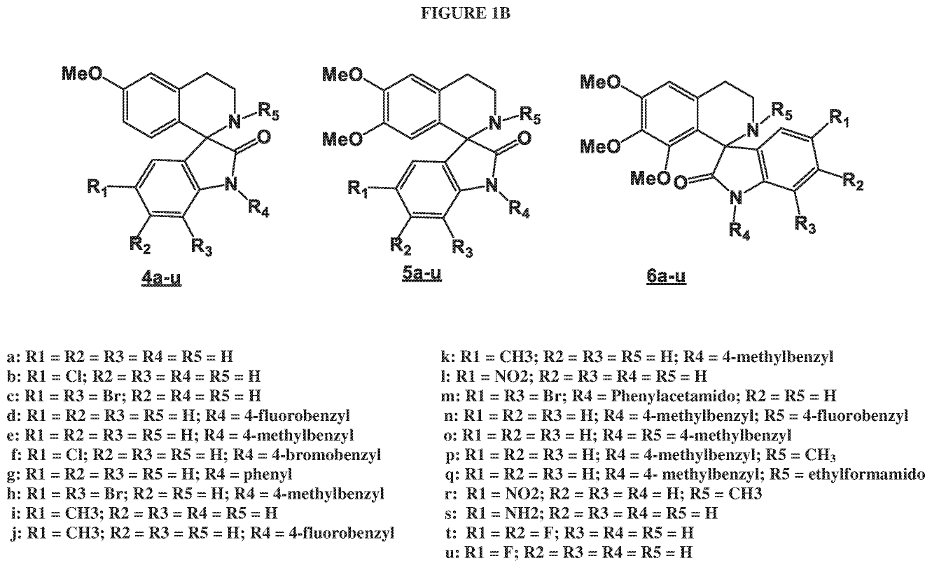

Representative Compounds of this Invention which are Presented in FIG. 1 were tested for 1) Anticancer Activity against a Panel of 60 Cancer Lines; 2) Antiproliferative Activity on EMT Breast Cancer Cells; and 3) for Binding to Sphingosine-1-phosphate (SIP) 1 Receptors.

Screening for Anticancer Activity—The NCI-60 panel: Representative compounds of the invention were subjected to anticancer testing in the National Cancer Institute (NCI) 60 screen. In this screen compounds are tested on a panel of sixty human cancer cell lines chosen from the following cancers: leukemia, nonsmall cell lung cancer (NSCLC), colon, melanoma, central nervous system (CNS), ovarian, renal, prostate and breast. The assay method is found on the NCI website (dtp.cancer.gov/discovery_development/nci-60/methodology.htm). In the NCI-60 assay, test compounds undergo preliminary screening at 10 μM concentration. The data is reported as percent growth relative to “no drug” control; therefore values between 0 and 100% denote growth inhibition. For instance, 65% growth denotes 35% inhibition of growth cell growth.

Weak to moderate antiproliferative activity was observed on a number of cancer cell lines, notably nonsmall cell lung cancer (NSCLC), leukemia, renal cancer, lung cancer, ovarian, prostate and breast cancer. The noteworthy compounds include 1e, 2c,g,h, 4b,c, 5a,c,e,f and 6b; moreover, three compounds 4e, 5e and 5f appeared to be the most potent analogues (Table 1, below). Therefore, anticancer activity resides in the dihydrospiro[indoline-1′:3-isoquinolin]-2-one hybrid skeleton. Representative compounds of the invention were tested on all cell lines in the NCI-60 panel. However, the only cell lines shown are those that were inhibited by at least 30 percent. Table 1, below, presents the data along cancer types while Table 1A identifies the specific cell lines affected.

TABLE 1

| | Percent (%) Inhibition of Cancer Cell Line | | | | | | | |
|---|---|---|---|---|---|---|---|---|
| | | | Renal | | | | | |
| Cpd No. | NSCLC | Leukemia | Cell | CNS | Melanoma | Ovarian | Prostate | Breast |
| 1a | 15 | | 14 | | | 10 | | |
| 1e | 30 | | 35 | | | 14 | 14 | 12 |
| 2b | 17 | | 17 | | | | | |
| 2c | | | 25 | | | 25-37 | | 23 |
| 2g | 25 | 19 | 22-36 | | | | 19 | 13 |
| 2h | 32 | 24 | 25 | | | | | |
| 4a | | | 20 | 20 | | | | |
| 4b | | | 23-30 | | | | | |
| 4e | 25-28 | | 30-46 | | | | | 25-30 |
| 5a | | | 25-37 | | | | | |
| 5b | | | 22 | 32 | | | | |
| 5c | | 24 | 27-35 | 25 | | | | |
| 5e | | 24-48 | 25 | | | | 42 | 22-24 |
| 5f | | 38-55 | | | 44 | 39 | 52 | 27-36 |
| 6a | | 22 | 22 | | | | | |
| 6b | 20 | | 28-31 | | | 20 | | |

Anticancer Screening on EMT6 Cancer Cells

Representative compounds were screened tested for antiproliferative activity on the EMT6 breast cancer cell line. Screening was carried out according to published procedures (Zeng et al., 2014). In this screen, compounds were tested at 100 μM concentration. Out of twenty-two compounds, at least five showed some evidence of antiproliferative activity; and three of them, 2f, 2g and 2l, were found to inhibit cell proliferation by as much as fifty percent.

TABLE 2

| | EMT6 cell proliferation (%) | SD |
|---|---|---|
| no ligand | 100 | 1.13 |
| 1a | 104.22 | 5.32 |
| 1c | 87.56 | 0.81 |
| 1d | 119.54 | 3.03 |
| 1e | 77.91 | 1.55 |
| 1f | 103.29 | 3.20 |
| 1g | 91.07 | 2.82 |
| 1h | 126.83 | 6.07 |
| 1i | 108.20 | 4.91 |
| 2b | 108.69 | 3.62 |
| 2c | 94.93 | 1.87 |
| 2d | 123.61 | 3.85 |
| 2e | 72.16 | 2.46 |
| 2f | 41.42 | 1.29 |
| 2g | 56.49 | 4.34 |
| 2h | 132.08 | 2.67 |
| 2i | 98.47 | 2.33 |
| 2l | 37.24 | 5.88 |
| 2m | 72.26 | 4.68 |
| 3a | 91.17 | 1.67 |
| 4b | 142.25 | 10.05 |
| 2k | 130.94 | 2.75 |

The binding of a representative group of compounds of this invention to sphingosine-1-phosphate receptor 1 (S1PR1) was determined by a published procedure (Rosenberg et al., 2016). In this assay, the test compound at 100 nM concentration was incubated with 0.2 nM radioligand. Active compounds or “hits” are defined as compounds that inhibit radioligand binding by 50% or more. Out of 50 tested, eleven compounds, 1d, 1f, 1h, 1k, 1l, 1o, 2d, 2l, 2o, 5b and 6d were found to inhibit ligand binding by 50% or more. This finding is significant because it establishes the 3′,4′-dihydro-2′H-spiro[indoline-1:3′-isoquinolin]-2-one hybrid scaffold as an entirely new class of ligands for the sphingosine-1-phosphate receptors.

TABLE 3
Inhibition of binding to the SIP1 Receptor

| COMPOUND | % INHIBITION |
|---|---|
| 1d | 57.8 |
| 1f | 55.4 |
| 1h | 54.8 |
| 1i | 35.4 |
| 1k | 50.9 |
| 1l | 60.0 |
| 1o | 50.9 |
| 2d | 59.5 |
| 2l | 56.1 |
| 2m | 52.4 |
| 3b | 34.2 |
| 4a | 36.2 |
| 4c | 36.3 |
| 4d | 41.5 |
| 4e | 38.1 |
| 5a | 39.1 |
| 5b | 50.4 |
| 5c | 37.6 |
| 5d | 40.0 |
| 5e | 32.4 |
| 6a | 38.4 |
| 6b | 41.1 |
| 6c | 36.7 |
| 6d | 50.2 |
| 6e | 31.3 |
| 6f | 36.5 |

The invention will now be illustrated by the following nonlimiting experiments. Unless otherwise stated, the following will apply:

General

All chemicals were purchased from Sigma Aldrich Chemicals Company, St. Louis, Mo., USA and were used as supplied. All solvents were reagent grade. Where necessary, solvents and starting materials were purified using standard procedures. Solvent removal was performed in vacuo using a Buchi rotatory evaporator at temperatures not greater than 60° C. Melting points were measured using the Mel-Temp II apparatus with the use of open capillaries and are uncorrected.

Chromatography

The progress of all reactions was monitored using thin layer chromatography (TLC) on aluminum backed sheets of Sigma Aldrich silica gel 60 F₂₅₄ plates. Compounds were visualized under UV light at 254 nm or in an iodine chamber. Chromatographic purification of compounds was carried out by Medium Pressure Liquid Chromatography (MPLC) using silica gel 60-400 mesh. The solvent mixtures used in specific chromatographic runs are indicated where necessary.

Mass Spectrometry

High resolution Fourier transform mass spectrometry electrospray ionisation (FTMS-ESI) mass spectra were carried out on an LTQ Orbitrap XL mass spectrometer from Thermo Fisher Scientific (Bremen, Germany). A heated electrospray interface (H-ESI) was operated for ionization of the molecules at a spray voltage of 5 kV, whereas capillary voltage and tube lens voltages were adjusted to 20 and 100 V, respectively. The vaporizer temperature was set at 250° C. and the ion transfer capillary temperature to 200° C. Measurements were carried out in the positive ion mode in a mass range of m/z 100-600 at a mass resolution of 60,000 at m/z 200. MS/MS experiments were performed using argon as collision gas in collision-induced dissociation (CM) mode, collision energies were measured at 15 eV, 25 eV and 35 eV.

Nuclear Magnetic Resonance (NMR) Spectroscopy

Nuclear magnetic resonance spectra were obtained using a Brucker Avance III spectrometer operating at 600 MHz (H¹) and 150 MHz (¹³C). Spectra were recorded in deuterated solvents as indicated in brackets and referenced to residual solvent signals. Chemical shifts (δ) were measured in parts per million (ppm). Hydrogen and carbon peak assignments were performed using gradient correlation spectroscopy (gCOSY), heteronuclear single quantum correlation (gHSQC) spectroscopy, and heteronuclear multiple bond correlation (gHMBC) techniques. For most of the compounds, signals of protons attached to oxygen and nitrogen were not observed and were attributed to exchange with solvent protons. Signal multiplicities are reported as singlet (s), doublet (d), doublet of doublets (dd), doublet of triplets (dt), triplet (t), triplet of doublets (td) and multiplet (m). Coupling constants (J) are reported in Hertz (Hz) units.

### EXAMPLES

### Example 1

Synthesis of 3-hydroxyphenethylamine

This compound was synthesized following the method of Ngo-Hanna et al., 2014. In a typical experiment, a mixture of 3-methoxyphenethylamine (1.0 g, 6.6 mmol), hydrobromic acid (10 mL) and acetic acid (10 mL) was stirred and heated under reflux for six (6) hours. The cooled mixture was concentrated under reduced pressure to obtain a residue which was used in subsequent reactions without further purification; crude yield, 97%.

### Example 2

General Method for the Synthesis of Substituted N-benzylisatins (Method B)

This synthesis was performed using the method of Vine et al., 2007 with some modifications. The desired isatin (1 equiv) was taken up in anhydrous acetonitrile (1 mL per 0.1 mmol of isatin). Solid K₂CO₃ (1.2 equiv) was added in one portion, and the dark colored suspension was stirred at room temperature for 1 h. The appropriate benzyl halide (1.2 equiv) and KI (0.2 equiv) were added, and the reaction mixture was stirred at 80° C. for 5-18 h, until the isatin starting material had been consumed as revealed on TLC. The reaction mixture was decanted into HCl (0.5 M, 50 mL) and extracted with methylene chloride (20 mL×2), dried over anhydrous sodium sulfate and the solvent removed under reduced pressure. The crude product obtained was purified by flash chromatography using isocratic elution with hexane:ethyl acetate giving yellow to red crystals. Yields were between 80 to 95%.

### Example 3

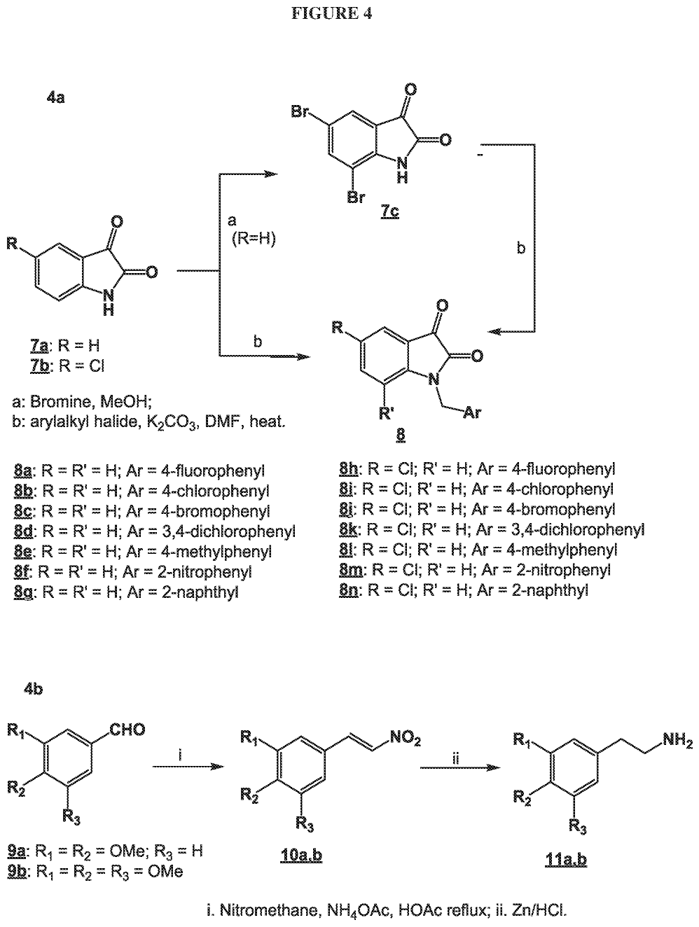

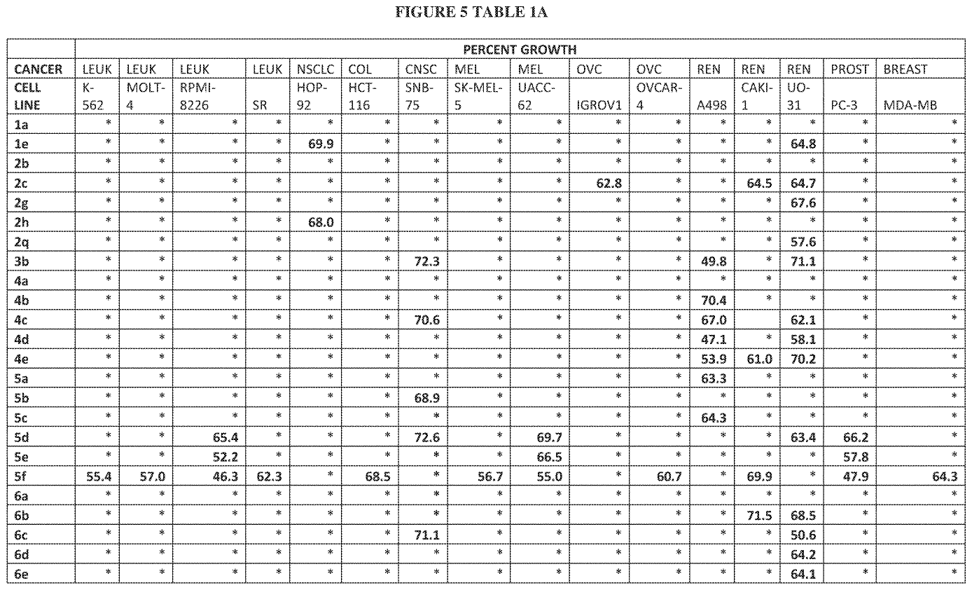

Synthesis of 5,7-Dibromoisatin (7c, FIG. 4).

The synthesis of 5,7-dibromoisatin was based on the method of Kumar et al., 2013. Isatin (9.0 g, 61.2 mmol, 1 equiv) was warmed in ethanol (95%, 100 mL) with stirring until it dissolved. Bromine (3.0 equiv, 16.3 g, 183.6 mmol, 9.4 mL) was added dropwise to the stirred isatin solution while maintaining the temperature of the reaction mixture between 70° C. and 75° C. The solution was cooled to room temperature and placed on ice for 30 min. The resulting precipitate was washed with water and cold ethanol and then recrystallized from ethanol to yield bright orange-red crystals of 5,7-dibromoisatin (66%), m.p. 248-250 (lit. 248-250° C.).

### Example 4

General Method for the Synthesis of 6′-hydroxy-3′,4′-dihydro-2′H-spiro[indoline-3,1′-isoquinolin]-2-ones (1a-q, FIG. 1) and 8′-hydroxy-3′,4′-dihydro-2′H-spiro[indoline-3,1′-isoquinolin]-2-ones (2a-q, FIG. 1). (Method C)

The compounds were synthesized by the phenolic Pictet-Spengler reaction reported by Kametani et al. (1968) and modified by Ngo-Hanna et al., (2014). In a typical experiment, crude 3-hydroxyphenethylamine (0.9 g, 6.6 mmol, 1 equiv), obtained as described in Example 1 above, and the appropriate isatin (1 equiv, 6.6 mmol) were dissolved in absolute ethanol (10 mL)and triethylamine (1 mL) was added. The reaction mixture was stirred and heated under reflux for 7 to 10 hours. Upon completion the reaction was concentrated under reduced pressure to remove the solvent. Distilled water was added to the viscous mass and the product which precipitated out was extracted into ethyl acetate (3×30 mL). The combined organic extracts were dried over anhydrous sodium sulphate and concentrated to minimum volume. The crude product was further purified by column chromatography using the appropriate solvent system. The final product obtained was recrystallized from absolute ethanol. In most cases, the reaction provided two regioisomers as earlier documented by Kametani et al. (1968), the 8-hydroxy compound which had a shorter retention time, and the 6′-hydroxy isomer. The combined yields ranged between 40 and 99%.

### Example 5

8′-Hydroxy-3′,4′-dihydro-2′H-spiro[indoline-3,1′-isoquinolin]-2-one (1a).

Method C. Synthesized from 3-hydroxyphenethylamine (Example 1) (0.9 g, 6.61 mmol, 1 equiv) and isatin (0.96 g, 6.61 mmol, 1 equiv). The crude product was purified on flash chromatography (hexane: ethyl acetate—60:40) and recrystallized from methanol. Yield 0.83 g, 45% (white solid). M.p. 256-258° C.

¹H NMR (DMSO-d₆, 700 MHz): δppm 2.80 (t, J=5.8 Hz, 2H, H4′a, H4′b), 3.07 dt, J=12.9, 6.3 Hz, 1H, H3′a), 3.13-3.16 (m, 1H, H3′b), 6.42 (dd, J=8.0, 1.2 Hz, 1H, H7′), 6.63 (dd, J=7.60, 1.1 Hz, 1H, H5′), 6.80 (ddd, J=6.0, 1.3 Hz, 2H, H5, H7), 6.88 (m, 1H, H4), 6.98 (t, J=7.7 Hz, 1H, H6′), 7.13 (td, J=7.6, 1.3 Hz, 1H, H6), 9.12 (s, 1H, 8′-OH), 10.19 (s, 1H, H1). ¹³C NMR (DMSO-d₆, 175 MHz): δppm 28.8 (C4′), 38.4 (C3′), 61.9 (C3/C1′), 108.8 (C7), 112.3 (C7′), 119. 6 (C5′), 120.6 (C5), 122.5 (C8′a), 123.1 (C4), 127.2 (C6′), 127.6 (C6), 135.6 (C3a), 138.4 (C4′a), 142.8 (C7a), 153.9 (C8′), 179.3 (C2). FTMS+cESI: m/z 267.11 [M+1]⁺

### Example 6

5-Chloro-6′-hydroxy-3′,4′-dihydro-2′H-spiro[indoline-3,1′-isoquinolin]-2-one (2b).

Method C. Prepared from 3-hydroxyphenethylamine (Example 1) (1.2 g, 6.61 mmol) and 5-chloroisatin (0.9 g, 6.61 mmol). The crude product was purified by chromatography on a short column (hexane: ethyl acetate—60:40) and recrystallized from methanol. Yield 1.0 g, 48% (white solid). M.p. 253-255° C.

¹H NMR (DMSO-d₆, 700 MHz): δppm 2.65 (dt, J=16.0, 3.4 Hz, 1H, H4′a), 2.86 (ddd, J=15.6, 9.3, 5.4 Hz, 1H, H4′b), 2.97 (dt, J=12.1, 4.7 Hz, 1H, H3′a), 3.05 (br s, 1H, H2′), 3.60 (ddd, J=12.8, 9.6, 4.1 Hz, 1H, H3′b), 6.24 (d, J=8.4 Hz, 1H, H8′), 6.42 (dd, J=8.4, 2.6 Hz, 1H, H7′), 6.55 (d, J−2.6 Hz, 1H, H5′), 6.89 (d, J=8.3 Hz, 1H, H7), 7.02 (d, J=2.2, 1H, H4), 7.26 (dd, J=8.3, 2.2 Hz, 1H, H6), 9.29 (s, 1H, 6′-OH), 10.37 (s, 1H, H1).

¹³C NMR (DMSO-d₆, 175 MHz): δppm 28.8 (C4′), 38.0 (C3′), 63.3 (C3/C1′), 110.9 (C7), 113.7 (C7′), 115.3 (C5′), 124.5 (C8′a), 125.0 (C4), 125.7 (C5), 126.9 (C8′), 128.3 (C6), 137.6 (C4′a), 138.1 (C3a), 141.1 (C7a). 156.0 (C6′), 180.0 (C2)

FTMS+cESI: m/z 301.07 [M+1]⁺

### Example 7

5,7-Dibromo-8′-hydroxy-3′,4′-dihydro-2′H-spiro[indoline-3,1′-isoquinolin]-2-one (1c).

Method C. Synthesized from 7c (1.5 g, 5 mmol, 1 equiv) and 3-hydroxyphenethylamine (Example 1)(0.81 g, 6 mmol). The product was separated from its 6′-OH regioisomer by column chromatography (hexane: ethyl acetate—80:20). Yield, 0.33 g, 16% (brown solid). M.p. 235-238° C.

¹H NMR (CD₃OD 600 MHz): δppm 2.95-2.92 (m, 2H, H4′a, H4′b), 3.16-3.22 (m, 1H, H3′a), 3.31 (d, J=5.2 Hz, 1H, H3′b), 6.53-6.55 (m, 1H, H7′), 6.74 (d, J=7.7 Hz, 1H, H5′), 7.06 -7.11 (m, 2H, H4, H6′), 7.56 (d, J−1.8, Hz, 1H, H6).

¹³C NMR (DMSO-d₆, 175 MHz): δppm 28.1 (C4′), 38.5 (C3′), 63.5 (C3/C1′), 102.5 (C7), 112.3 (C7′), 113.9 (C5), 120 (C5′), 120.2 (C8′a), 125.3 (C4), 128.2 (C6′), 132.8 (C6), 137.9 (C3a), 138.0 (C4′a), 141.4 (C7a), 154.1 (C8′), 179.9 (C2).

FTMS+cESI: m/z calcd for C₁₆H₁₃O₂N₂ ⁸¹Br₂ [M+1]⁺:426.9303; found 426.9303.

### Example 8

1-(4-Fluorobenzyl)-8′-hydroxy-3′,4′-dihydro-2H-spiro[indoline-3,1′-isoquinolin]-2-one (1d) and 1-(4-Fluorobenzyl)-6′-hydroxy-3′,4′-dihydro-2′H-spiro[indolline-3,1′-isoquinolin]-2-one (2d).

Method C. Prepared from 1-(4-fluorobenzyl)indoline-2,3-dione, 8a, (0.4 g, 1.6 mmol, 1 equiv) and 3-hydroxyphenethylamine (0.2 g, 1.6 mmol). The reaction afforded compounds 1d and 2d that were separated by column chromatography (hexane: ethyl acetate—80:20).

1d. Yield, 0.2 g, 33% (Brown solid). M.p. 119-121° C.

¹H NMR (CD₃OD, 600 MHz): δppm 2.93-3.06 (m, 2H, H4′a, H4′b), 3.32-3.37 (m, 2H, H3′a, H3′b), 4.84 (d, J=15.9 Hz, 1H, —CH₂—Ar), 5.16 (d, J=15.9 Hz, 1H, CH₂—Ar), 6.54 (dd, J=8.1, 1.1 Hz, 1H, H7′), 6.75-6.80 (m, 2H, H7, H5′), 6.97 (td, J=7.6, 1.0 Hz, 1H, H5), 7.07 (ddt, J=7.8, 4.0, 2.3 Hz, 4H, H4, H6′, H3″), 7.18 (td, J=7.7, 1.3 Hz, 1H, H6), 7.49-7.53 (m, 2H, H2″).

¹³C NMR (CD₃OD, 150 MHz): δppm 28.3 (C4′), 38.7 (C3′), 42.8 (—CH₂—Ar), 62.3 (C3/C1′), 108.9 (C7), 112.3 (C7′), 114.9 (2C, C3″), 120.8 (C5′), 120.8 (C8′a), 122.2 (C5), 123.2 (C4), 127.9 (2C, C6, C6′), 129.0 (2C, C2″), 132.1 (C1″), 133.7 (C3a), 138.0 (C4′a), 143.1 (C7a), 154.0 (C8′), 163.2 (C4″), 179.0 (C2).

FTMS+cESI: m/z 375.15 [M+1]⁺

2d. Yield, 0.2 g, 33% (brown solid). M.p. 220-222° C.

¹H NMR (CD₃OD, 600 MHz): δppm 2.88 (dt, J=16.4, 4.5, 1H, H4′a), 3.04 (ddd, J=15.2, 9.1, 5.5, 1H, H4′b), 3.18-3.23 (m, 1H, H3′a), 3.83-3.88 (m, 1H, H3′b), 4.90 (d, J=15.6 Hz, 1H, —CH₂—Ar), 5.01 (d, J=15.6 Hz, 1H, CH₂—Ar), 6.22 (d, J=8.5 Hz, 1H, H8′), 6.45 (dd, J=8.5, 2.6 Hz, 1H, H7′), 6.65 (d, J=2.6, 1H, H5′), 6.97 (d, J=7.9, 1H, H7), 7.06 (td, J=7.6, 1.0 Hz, 1H, H5), 7.07-7.11 (m, 2H, H2″, H6″), 7.19 (dd, J=7.5, 1.3 Hz, 1H, H4), 7.28 (td, J=7.8, 1.3 Hz, 1H, H6), 7.42-7.47 (m, 2H, H3″, H5″).

¹³C NMR (CD₃OD, 150 MHz): δppm 28.3 (C4′), 38.3 (C3′), 42.4 (—CH₂—Ar), 63.3 (C3/C1′), 109.2 (C7), 113.6 (C7′), 115.1 (2C, C3″, C5″), 115.2 (C5′), 123.2 (C5), 124.3 (C4), 124.9 (C8′a), 127.0 (C8′), 128.8 (C6), 129.2 (2C, C2″, C6″), 132.2 (C1″), 134.6 (C3a), 137.4 (C4′a), 142.5 (C7a), 156.4 (C6′), 163.2 (C4″), 179.2 (C2). FTMS+cESI: m/z 375.15 [M+1]⁺

### Example 9

1-(4-Chlorobenzyl)-8′-hydroxy-3′,4°-dihydro-2′H-spiro[indoline-3,1′-isoquinolin]-2-one (1e) and 1-(4-Chlorobenzyl)-6′-hydroxy-3′,4′-dihydro-2′H-spiro[indoline-3,1′-isoquinolin]-2-one (2e).

Method C. Prepared from 1-(4-chlorobenzyl)indoline-2,3-dione, 8b, (0.8 g, 3.0 mmol) and 3-hydroxyphenethylamine (0.6 g, 4.4 mmol). The regioisomeric products 1e and 2e were separated by column chromatography (hexane: ethyl acetate—80:20).

1e. Yield, 0.4 g, 24% (brown solid). M.p, 95-98° C. ¹H NMR (CD₃OD, 600 MHz): δppm 2.84 (dt, J=16.7, 4.9 Hz, 1H, H4′a), 2.90 (dt, J=16.7, 4.9 Hz, 1H, H4′b), 3.22 (m, 2H, H3′a, H3′b), 4.71 (d, J=16.1 Hz, 1H, —CH₂—Ar), 5.04 (d, J=16.1 Hz, 1H, C₂—Ar), 6.42 (dd, J=8.0, 1.1 Hz, 1H, H7′), 6.63-6.66 (m, 2H, H7, H5′), 6.85 (td, J=7.5, 1.0 Hz, 1H, H5), 6.93-6.97 (m, 2H, H4, H6), 7.06 (td, J=7.8, 1.3 Hz, 1H, H6′), 7.22-7.24 (m, 2H, H3″), 7.36 (d, J=8.4, 2H, H2″). ¹³C NMR (CD₃OD, 150 MHz): δppm 28.3 (C4′), 38.7 (C3′), 42.8 (—CH₂—Ar), 62.3 (C3/C1′), 109.0 (C7), 120.0 (2C, C5′, C7′), 120.7 (C8′a), 122.3 (C4), 122.3 (C5), 128.0 (C6′), 128.1 (C6), 128.3 (2C, C3″), 128.7 (2C, C2″), 132.8 (C4″), 133.6 (C3a), 135.0 (C1″) 137.9 (C4′a), 143.0 (C7a), 154.0 (C8′), 178.8 (C2). FTMS+cESI: m/z 391.12 [M+1]⁺

2e. Yield, 0.8 g, 47% (white solid). M.p. 218-220° C. ¹H NMR (CD₃OD, 600 MHz): δppm 2.88 (dt, J=16.4, 4.5, 1H, H4′a), 3.04(ddd, J=15.3, 9.2, 5.5 Hz 1H, H4′b), 3.18-3.24 (m, 1H, H3′a), 3.85 (ddd, J=12.7, 9.1, 4.6 Hz 1H, H3′b), 4.92 (d, J=15.8 Hz, 1H, —CH₂—Ar), 5.00 (d, J=15.8 Hz, 1H, CH₂—Ar), 6.23 (d, J=8.5 Hz, 1H, H8′), 6.46 (dd, J=8.5, 2.6 Hz, 1H, H7′), 6.66 (d, J=2.6, 1H, H5′), 6.94 (dd, J=7.9, 1H, H7), 7.06 (td, J=7.5, 1.0 Hz, 1H, H5), 7.19 (dd, J=7.5, 1.2 Hz, 1H, H4), 7.28 (td, J=7.7, 1.3 Hz, 1H, H6), 7.3-7.36 (m, 2H, H2″, H6″), 7.40 (m, 2H, H3″, H5″). ¹³C NMR (CD₃OD, 150 MHz): δppm 28.3 (C4′), 38.3 (C3′), 42.4 (—CH₂—Ar), 63.3 (C3/C1′), 109.2 (C7), 113.6 (C7′), 115.2 (C5′), 123.3 (C5), 124.3 (C4), 124.8 (C8′a), 127.0 (C8′), 128.5 (2C, C2″, C6″), 128.8 (C6), 129.1 (2C, C3″, C5″), 133.2 (C4″), 134.6 (C3a), 134.9 (C1″), 137.4 (C4′a), 142.5 (C7a), 156.4 (C6′), 179.2 (C2). FTMS+cESI: m/z 391.12 [M+1]⁺

### Example 10

Synthesis of 1-(4-bromobenzyl)-8′-hydroxy-3′,4′-dihydro-2′H-spiro[indoline-3,1′-isoquinolin]-2-one (1f) and 1-(4-bromobenzyl)-6′-hydroxy-3′,4′-dihydro-2′H-spiro[indoline-3,1′-isoquinolin]-2-one (2f).

Method C. Synthesized from 3-hydroxyphenethylamine (Example 1) (1.0 7g, 7.1 mmol) and 1-(4-bromobenzyl)indoline-2,3-dione, 8c, (1.5 g, 4.7 mmol). The regioisomers 1f and 2f were separated by column chromatography (hexane: ethyl acetate—80:20).

1f. Yield, 0.3 g, 15% (brown solid). M.p. 81-83° C.

¹H NMR (CD₃OD, 600 MHz): δppm 2.97-3.01 (m, 2H, H4′a, H4′b), 3.34 (m, 2H, H3′a, H3′b), 4.81 (d, J=16.3 Hz, 1H, —CH₂—Ar), 5.14 (d, J=16.3 Hz, 1H, CH₂—Ar), 6.54 (dd, J=8.0, 1.1 Hz, 1H, H7′), 6.75-6.78 (m, 2H, H7, H5′), 6.97 (td, J=7.5, 1.0 Hz, 1H, H5), 7.07-7.09 (m, 1H, H4), 7.19 (td, J=7.8, 1.3 Hz, 1H, H6′), 7.29-7.31 (m, H6), 7.41-7.44 (m, 2H, H3″), 7.50 (m, 2H, H2″). ¹³C NMR (CD₃OD, 150 MHz): δppm 28.2 (C4′), 38.7 (C3′), 42.8 (—CH₂—Ar), 62.3 (C3/C1′), 109.0 (C7), 112.3 (C7′), 114.6 (C4″), 120.0 (C5′), 120.7 (C8′a), 122.3 (C5), 123.3 (C4), 128.1 (C6′), 128.7 (C6), 129.0 (2C, C3″), 131.3 (2C, C2″), 133.9 (C1″), 135.5 (C3a), 137.9 (C4′a), 143.0 (C7a), 154.0 (C8′), 178.8 (C2).

FTMS+cESI: m/z 435.07 [M+1]⁺

2f. Yield, 0.82 g, 40% (white solid). M.p. 207-210° C.

¹H NMR (CD₃OD, 600 MHz): δppm 2.88 (dt, J=16.4, 4.5, 1H, H4′a), 3.04 (ddd, J=15.2, 9.2, 5.5 Hz 1H, H4′b), 3.17-3.24 (m, 1H, H3′a), 3.82-3.89 (m, 1H, H3′b), 4.90 (d, J=15.8 Hz, 1H, —CH₂—Ar), 4.98 (d, J=15.8 Hz, 1H, CH₂—Ar), 6.24 (d, J=8.5 Hz, 1H, H8′), 6.46 (dd, J=8.5, 2.6 Hz, 1H, H7′), 6.66 (d, J=2.6, 1H, H5′), 6.94 (dd, J=7.8, 1H, H7), 7.06 (td, J=7.5, 1.0 Hz, 1H, H5), 7.19 (dd, J=7.5, 1.3 Hz, 1H, H4), 7.28 (td, J=7.8, 1.3 Hz, 1H, H6), 7.33-7.36 (m, 2H, H2″, H6″), 7.50-7.54 (m, 2H, H3″, H5″).

¹³C NMR (CD₃OD, 150 MHz): δppm 28.3 (C4′), 38.3 (C3′), 42.4 (—CH₂—Ar), 63.3 (C3/C1′), 109.2 (C7), 113.6 (C7′), 115.2 (C5′), 121.1 (C4″), 123.3 (C5), 124.3 (C4), 124.8 (C8′a), 127.1 (C8′), 128.8 (C6), 129.1 (2C, C3″, C5″), 131.5 (2C, C2″, C6″), 134,6 (C3a), 134.6 (C1″), 137.4 (C4′a), 142.5 (C7a), 156.4 (C6′), 179.2 (C2).

FTMS+cESI: m/z 435.07 [M+1]⁺

### Example 11

1-(3,4-Dichlorobenzyl)-8′-hydroxy-3′,4′-dihydro-2′H-spiro[indoline-3,1′-isoquinolin]-2-one (1g) and 1-(3,4-Dichlorobenzyl)-6′-hydroxy-3′,4′-dihydro-2′H-spiro[indoline-3,1′-isoquinolin]-2-one (2g).

Method C. Prepared from 3-hydroxyphenethylamine (Example 1) (0.82 g, 6.0 mmol) and 1-(3,4-dichlorobenzyl)indoline-2,3-dione, 8d, (1.22 g, 4.0 mmol). The reaction afforded compounds 1g and 2g that were separated by column chromatography (hexane: ethyl acetate—80:20).

1g. Yield, 0.4 g, 24% (brown solid). M.p. 109-113° C.

¹H NMR (CD₃OD, 600 MHz): δppm 2.93-3.06 (m, 2H, H4′a, H4′b), 3.27-3.37 (m. 2H, H3′a, H3′b), 4.85 (d, J=16.3 Hz, 1H, —CH₂—Ar), 5.12 (d, J=16.3 Hz, 1H, CH₂—Ar), 6.54 (dd, J=8.0, 1.1 Hz, 1H, H7′), 6.76 (dd, J=7.6, 1.2 Hz, 1H, H5′), 6.81 (d, J=7.8 Hz, 1H, H7), 6.99 (td, J=7.5, 1.0 Hz, 1H, H5), 7.05-7.10 (m, 2H, H4, H6′), 7.22 (td, J=7.7, 1.3 Hz, H6), 7.42 (dd, J=8.3, 2.1 Hz, 1H, H6″), 7.49 (d, J=8.3, 1H H5″), 7.70 (d, J=2.1 Hz, 1H, H2″).

¹³C NMR (CD₃OD, 150 MHz): δppm 28.3 (C4′), 38.7 (C3′), 42.4 (—CH₂—Ar), 62.2 (C3/C1′), 108.8 (C7), 114.4 (C7′), 120.0 (C5′), 120.8 (C8′a), 122.4 (C5), 123.3 (C4), 127.0 (C6″), 127.9 (C6′), 128.1 (C6), 129.3 (C2″), 130.3 (C5″), 130.9 (C3′), 132.1 (C4″), 133.8 (C3a), 137.2 (C1″), 138.0 (C4′a), 142.9 (C7a), 154.0 (C8′), 179.0 (C2).

FTMS+cESI: m/z 425.08 [M+1]⁺

2g. Yield, 0.9 g, 53% (white solid). M.p. 197-199° C.

¹H NMR (CD₃OD, 600 MHz): δppm 2.88 (dt, J=16.4, 4.4, 1H, H4′a), 3.00-3.08 (m, 1H, H4′b), 3.20 (ddd, J=12.8, 5.5, 4.3 Hz 1H, H3′a), 3.86 (ddd, J=12.8, 9.3. 4.6 Hz 1H, H3′b), 4.90 (d, J=16.0 Hz, 1H, —CH₂—Ar), 5.00 (d, J=16.0 Hz, 1H, CH₂—Ar), 6.23 (d, J=8.5 Hz, 1H, H8′), 6.47 (dd, J=8.5. 2.6 Hz, 1H, H7′), 6.66 (d, J=2.6, 1H, H5′), 6.97 (d, J=7.9, 1H, H7), 7.08 (td, J=7.5 , 1.0 Hz, 1H, H5), 7.21 (dd, J=7.5, 1.3 Hz, 1H, H4), 7.28 (td, J=7.8, 1.3 Hz, 1H, H6), 7.35 (dd, J=8.3, 2.1 Hz, 1H, H6″) 7.52 (d, J=8.3, 1H, H5″), 7.59 (d, J=2.1, 1H, H2″),

¹³C NMR (CD₃OD, 150 MHz): δppm 28.3 (C4′), 38.3 (C3′), 41.9 (—CH₂—Ar), 63.2 (C3/C1′), 109.0 (C7), 113.7 (C7′), 115.2 (C5′), 123.4 (C5), 124.4 (C8′a), 124.8 (C4), 12.7 (C8′), 127.1 (C6″), 128.9 (C6), 129.2 (C2″), 130.5 (C5″), 131.2 (C4″), 132.3 (C1′), 134.5 (C3a), 137.1 (C3″), 137.4 (C4′a), 142.3 (C7a), 156.4 (C6′), 179.2 (C2). FTMS+cESI: m/z 425.08 [M+1]⁺

### Example 12

8′-Hydroxy-1-(4-methylbenzyl)-3′,4′-dihydro-2′H-spiro[indoline-3,1′-isoquinolin]-2-one (1h) and 6′-Hydroxy-1-(4-methylbenzyl)-3′,4′-dihydro-2′H-spiro[indoline-3,1′-isoquinolin]-2-one (2h).

Method C. Prepared from 1-(4-methylbenzyl)indoline-2,3-dione, 8e, (053 g, 2.0 mmol) and 3-hydroxyphenethylamine (Example 1) (0.4 g, 2.8 mmol). The products 1h and 2h were separated by column chromatography (hexane: ethyl acetate 80:20).

1h. Yield, 0.1 g, 12% (brown solid). M.p. 110-112° C. ¹H NMR (CD₃OD, 600 MHz): δppm 2.33 (s, Ar—CH₃), 2.96 (dt, J=16.4, 4.7 Hz, 1H, H4′a), 3.03 (dt, J=16.4, 4.9 Hz, 1H, H4′b), 3.34 (m, 2H, H3′a, H3′b), 4.83 (d, J=15.8 Hz, 1H, —CH₂—Ar), 5.1 (d, J=15.8 Hz, 1H, CH₂—Ar), 6.54 (dd, J=8.0, 1.1 Hz, 1H, H7′), 6.74-6.78 (m, 2H, H7, H5′), 6.95 (td, J=7.5, 1.0 Hz, 1H, H5), 7.05-7.09 (m, 2H, H4, H6′), 7.16-7.18 (m, 3H, H6, 2H2″). ¹³C NMR (CD₃OD, 150 MHz): δppm 19.8 (Ar—CH₃), 28.3 (C4′), 38.7 (C3′), 43.4 (—CH₂—Ar), 62.3 (C3/C1′), 109.2 (C7), 112.3 (C7′), 119.9 (2C, C5′), 120.9 (C8′a), 122.1 (C5), 123.1 (C4), 127.0 (2C, C2″), 127.9 (2C, C6,C6′), 128.8 (2C, C3″), 1330 (C1″), 133.7 (C3a), 136.7 (C4″), 137.9 (C4′a), 143.3 (C7a), 154.1 (C8′), 179.0 (C2). FTMS+cESI: m/z 371.17 [M+1]⁺

2h. Yield, 0.5 g, 62%. M.p. 190-193° C. (White solid). ¹H NMR (CD₃OD, 600 MHz): δppm 2.33 (s, 3H, Ar—CH₃), 2.88 (dt, J=16.4, 4.6, 1H, H4′a), 3.03 (ddd, J=16.4, 9.0, 5.5 Hz 1H, H4′b), 3.20 (dt, J=12.8, 5.1 Hz H3′a), 3.84 (ddd, J=12.8, 9.1, 4.6 Hz 1H, H3′b), 4.82 (d, J=15.6 Hz, 1H, —CH₂—Ar), 5.01 (d, J=15.6 Hz, 1H, CH₂—Ar), 6.24 (d, J=8.5 Hz, 1H, H8′), 6.45 (dd, J=8.5, 2.6 Hz, 1H, H7′), 6.65 (d, J=2.6, 1H, H5′), 6.93 (dd, J=7.9, 1H, H7), 7.03 (td, J=7.5, 1.0 Hz, 1H, H5), 7.17 (m, 3H, H4, H3″, H5″), 7.25 (td, J=7.8, 1.3 Hz, 1H, H6), 7.28 (d, 2H, J=8.0, H2″, H6″). ¹³C NMR (CD₃OD, 150 MHz): δppm 28.3 (C4′), 38.3 (C3′), 42.9 (—CH₂—Ar), 63.3 (C3/C1′), 109.4 (C7), 113.6 (C7′), 115.1 (C5′), 123.1 (C5), 124.2 (C4), 124.9 (C8′a), 127.1 (3C, C8′, C2″, C6″), 128.7 (C6), 129.0 (2C, C3″, C5″), 133.0 (C1″), 134.6 (C3a), 137.2 (C4″), 137.3 (C4′a), 142.7 (C7a), 156.3 (C6′), 179.2 (C2). FTMS+cESI: m/z 371.17 [M+1]⁺

### Example 13

8-Hydroxy-1-(2-nitrobenzyl)-3′,4′-dihydro-2′H-spiro[indoline-3,1′-isoquinolin]-2-one (1i) and 6′-Hydroxy-1-(2-nitrobenzyl)-3′,4′-dihydro-2′H-spiro[indoline-3,1′-isoquinolin]-2-one (2i).

Method C. Synthesized from (2-nitrobenzyl)indoline-2,3-dione, 8f, (0.8 g, 3.0 mmol) and 3-hydroxyphenethylamine (0.6 g, 4.3 mmol). The reaction afforded two regioisoniers, 1i and 2i that were separated by column chromatography (hexane: ethyl acetate—80:20).

1i. Yield, 0.1 g, 8% (brown solid). M.p. 122-124° C. ¹H NMR (CD₃OD, 600 MHz): δppm 2.94-3.04 (m, 2H, H4′a, H4′b), 3.36 (m, 2H, H3′a, H3′b), 5.27 (d, J=18.0 Hz, 1H, —CH₂—Ar), 5.49 (d, J=18.0 Hz, 1H, CH₂—Ar), 6.57 (dd, J=8.0, 1.1 Hz, 1H, H7′), 6.70 (d, J=7.9 Hz, 1H, H7), 6.78 (dd, J=7.6 Hz, 1H, H5′), 7.02 (td, J=7.5, 1.0 Hz, 1H, H5), 7.09 (td, J=7.8 Hz, 1H, H6′), 7.13 (dd, J=7.4, 1.3 Hz, 1H, H4), 7.21 (td, J=7.8, 1.3 Hz, 1H, H6), 7.55 (td, J=7.7, 1.4 Hz, 1H, H6′), 7.65 (td, J=7.6, 1.3 Hz, 1H, H6″), 8.22 (dd, J=8.2, 1.3 Hz, 1H, H3″). ¹³C NMR (CD₃OD, 150 MHz): δppm 28.3 (C4′), 38.6 (C3′), 41.5 (—CH₂—Ar), 62.3 (C3/C1′), 108.7 (C7), 112.3 (C7′), 120.1 (C5′), 120.7 (C8′a), 122.6 (C5), 123.4 (C4), 124.9 (C3″), 128.0 (C6′), 128.1 (C4″), 128.2 (2C, C6, C6″), 131.8 (C1″), 133.6 (C3a), 133.7 (C5″), 138.1 (C4′a), 142.9 (C7a), 148.0 (C2″), 153.9 (C8′), 179.3 (C2). FTMS+cESI: m/z 402.14 [M+1]⁺

2i. Yield, 0.6 g, 50% (reddish brown solid). M.p. 115-118° C. ¹H NMR (CD₃OD, 600 MHz): δppm 2.88 (dt, J=16.5, 4.4, 1H, H4′a), 3.06 (ddd, J=15.3, 9.3, 5.6 Hz 1H, H4′b), 3.22 (ddd, J=12.8, 9.3, 4.6 Hz 1H, 1H, H3′a), 3.87 (ddd, J=12.8, 9.3, 4.6 Hz 1H, H3′b), 5.30-5.39 (m 2H, —CH₂—Ar), 6.39 (d, J=8.5 Hz, 1H, H8′), 6.51 (dd, J=8.5, 2.6 Hz, 1H, H7′), 6.66 (d, J=2.6, 1H, H5′), 6.83 (dd, J=7.9, 1H, H7), 7.10 (td, J=7.5, 1.0 Hz, 1H, H5), 7.25 (dd, J=7.5, 1.2 Hz, 1H, H4), 7.28 (td, J=7.8, 1.3 Hz, 1H, H6), 7.55 (td, J=7.8, 1.4 Hz, 1H H4″), 7.65 (td, J=7.6, 1.4 Hz, 1H, H5″), 8.19 (dd, J=8.20, 1.4 Hz, 1H, H3″). ¹³C NMR (CD₃OD, 150 MHz): δppm 28.2 (C4′), 38.3 (C3′), 40.8 (—CH₂—Ar), 63.4 (C3/C1′), 109.0 (C7), 113.7 (C7′), 115.2 (C5′), 123.3 (C5), 124.5 (C4), 124.7 (C8′a), 124.5 (C3″), 127.2 (C8′), 127.6 (C6″), 128.3 (C4″), 129.1 (C6), 131.2 (C1″), 134.4 (C3a), 133.7 (C5″), 137.4 (C4′a), 142.5 (C7a), 148.3 (C2″), 156.5 (C6′), 179.4 (C2). FTMS+cESI: m/z 402.14 [M+1]⁺

### Example 14

8′-Hydroxy-1-(naphthalen-2-ylmethyl)-3′,4′-dihydro-2′H-spiro[indoline-3,1′-isoquinolin]-2-one (1j) and 6′-Hydroxy-1-(naphthalen-2-ylmethyl)-3′,4′-dihydro-2′H-spiro[indoline-3,1′-isoquinolin]-2-one (2j).

Method C. Prepared from 1-(naphthalen-2-ylmethyl)indoline-2,3-dione, 8g, (2.7 g, 9.4 mmol) and 3-hydroxyphenethylamine (1.54 g, 11.3 mmol). The reaction afforded compounds, 1j and 2j, that were separated by column chromatography (hexane: ethyl acetate—80:20).

1j. Yield, 0.1 g, 16% (brown solid). M.p. 127-129° C. ¹H NMR (CD₃OD 600 MHz): δppm 2.97 (dt, J=16.6, 4.8 Hz, 1H, H4′a,), 3.0-3.07 (m, 1H, H4′b), 3.36 (m, 2H, H3′a, H3′b), 5.05-5.08 (m, 1H, —CH₂—Ar), 5.29 (dd, J=16.0 Hz, 1H, CH₂—Ar), 6.58 (dd, J=8.0, 1.1 Hz, 1H, H7′), 6.77 (dd, J=7.6, 1.1 Hz, 1H, H5′), 6.82 (dd, J=7.9, 1.1 Hz, 1H, H7), 6.95 (td, J=7.5, 1.0 Hz, 1H, H5), 7.08-7.11 (m, 2H, H4, H6′), 7.13-7.16 (m, 1H, H6), 7.47(td, J=7.4, 6.7, 3.4 Hz, 2H, H6″, H7″), 7.60 (dd, J=8.5 Hz, 1H, H3″), 7.83-7.90 (m, 3H, H4″, H5″, H8″), 7.97 (s, 1H, H2″). ¹³C NMR (CD₃OD, 150 MHz): δppm 28.2 (C4′), 38.7 (C3′), 43.8 (—CH₂—Ar), 62.4 (C3/C1′), 109.4 (C7), 112.2 (C7′), 120.0 (C5′), 122.2 (C8′a), 122.6 (C5), 123.3 (C4), 125.1 (C3″), 125.5 (C6″), 125.6(C1″) 125.8 (C7″), 127.2 (C8″), 127.5 (C5″), 128.0 (C4″), 128.1 (C67), 128.1(C8″a), 129.3 (C6), 132.9 (C4″a), 133.5 (C2″), 133.6 (C3a), 137.8 (C4′a), 143.4 (C7a), 154.1 (C8′), 179.0 (C2). FTMS+cESI: m/z 407.18 [M+1]⁺

2j. Yield, 1.5 g, 36% (brown solid). Mp 148-150° C. ¹H NMR (CD₃OD, 600 MHz): δppm 2.90 (dt, J=16.4, 4.6 Hz, 1H, H4′a), 3.05 (ddd, J=15.1, 9.0, 5.5 Hz, 1H, H4′b), 3.23 (dt, J=12.8, 5.0 Hz, 1H, H3′a), 3.89 (ddd, J=13.2, 9.0, 4.6 Hz, 1H, H3′b), 5.05 (d, J=15.7 Hz, 1H, —CH₂—Ar), 5.21 (d, J=15.7 Hz, 1H, CH₂—Ar), 6.30 (d, J=8.5 Hz, 1H, H8′), 6.47 (dd, J=8.5, 2.6 Hz, 1H, H7′), 6.67 (d, J=2.6, 1H, H5′), 6.99 (d, J=7.9, 1H, H7), 7.20 (dd, J=7,4, 1.2, 1H, H4), 7.23 (td, J=7.8, 1.2 Hz, 1H, H6) 7.45-7.52 (m, 3H, H3″, H6″, H7″), 7.82-7.87 (m, 3H, H4″, H5″, H8″), 7.89 (d, J=1.8 Hz, 1H, H1″). ¹³C NMR (CD₃OD, 150 MHz): δppm 28.3 (C4″), 38.4 (C3′), 43.3 (—CH₂—Ar), 63.4 (C3/C1′), 109.4 (C7), 115.2 (C5′), 123.2 (C5), 124.3 (C4), 124.9 (C8′a), 125.0 (C7″), 125.7 (C6″), 125.9 (C1″), 126.0 (C3″), 127.1 (C8′), 127.3 (C8″), 127.4 (C5″), 128.1 (C8″a), 128.8 (C6), 133.0 (C3a), 133.5 (C2″), 134.6 (C4″a), 137.4 (C4′a), 142.7 (C7a), 156.4 (C6′), 179.3 (C2). FTMS+cESI: m/z 407.18 [M+1]⁺

### Example 15

5-Chloro-1-(4-fluorobenzyl)-8′-hydroxy-3′,4′-dihydro-2′H-spiro[indoline-3,1′-isoquinolin]-2-one (1k) and 5-Chloro-1-(4-fluorobenzyl)-6′-hydroxy-3′,4′-dihydro-2′H-spiro[indoline-3,1′-isoquinolin]-2-one (2k).

Method C. Synthesized from 5-chloro-1-(4-fluorobenzyl)indoline-2,3-dione, 8h, (1.46 g, 5.0 mmol, 1 equiv) and 3-hydroxyphenethylamine (0.7 g, 5.0 mmol). The reaction products 1k and 2k were separated by column chromatography (hexane: ethyl acetate—80:20).

1k. Yield, 0.7 g, 35% (brown solid). M.p. 120-123° C. ¹H NMR (CD₃OD, 600 MHz): δppm 2.97 (m, 2H, H4′a, H4′b), 3.21-3.28 (m, 1H, H3′a), 3.36 (dd, J=13.0, 5.3 Hz, 1H, H3′b), 4.81 (d, J=15.9 Hz, 1H, —CH₂—Ar), 5.14 (d, J=15.9 Hz, 1H, CH₂—Ar), 6.56 (dd, J=8.1, 1.1 Hz, 1H, H7′), 6.75 (d, J=8.4 Hz, 1H, H7), 6.77 (dd, J=7.6, 1.1 Hz, 1H, H5′), 7.04 (d, J=2.12 Hz, 1H, H4), 7.09 (ddd, J=11.3, 9.1, 7.0 Hz, 3H, H6′, H3″), 7.18 (dd, J=8.4, 2.1 Hz, 1H, H6), 7.47-7.51(m, 2H, H2″). ¹³C NMR (CD₃OD, 150 MHz): δppm 28.3 (C4′), 38.7 (C3′), 42.8 (—CH₂—Ar), 62.3 (C3/C1′), 110.0 (C7), 112.3 (C7′), 114.9/115.0 (2C, C3″), 120.1 (C5′), 123.4 (C4), 127.4 (C8′a), 127.7(C6), 128.2 (C6′), 129.0 (3C, C3a, C2″), 131.7 (C1″), 135.8 (C5), 138.2 (C4′a), 141.8 (C7a), 154.0 (C8′) 163.2 (C4″), 178.6 (C2). FTMS+cESI: m/z 409.11 [M+1]⁺

2k. Yield, 1.5 g, 74% (brown solid). M.p. 208-210° C. ¹H NMR (CD₃OD, 600 MHz): δppm 2.86 (dt, J=16.4, 4.4, 1H, H4′a), 2.99-3.07 (m, 1H, H4′b), 3.18 (ddd, J=12.7, 5.1, 4.2 Hz, 1H, H3′a), 3.85 (ddd, J=12.7, 9.3, 4.5 Hz 1H, H3′b), 4.86 (d, J=15.7 Hz, 1H, —CH₂—Ar), 4.99 (d, J=15.7 Hz, 1H, CH₂—Ar), 6.23 (d, J=8.5 Hz, 1H, H8′), 6.48 (dd, J=8.5, 2.6 Hz, 1H, H7′), 6.67 (d, J=2.6, 1H, H5′), 6.94 (d, J=8.4, 1H, H7), 7.07-7.12 (m, 2H, H3″, H5″), 7.19 (d, J=2.1 Hz, 1H, H4), 7.28 (dd, J=8.4, 2.3 Hz, 1H, H6), 7.40-7.44 (m, 2H, H2″, H6″). ¹³C NMR (CD₃OD, 150 MHz): δppm 28.2 (C4′), 38.3 (C3′), 42.4 (—CH₂—Ar), 63.3 (C3/C1′), 110.4 (C7), 113.8 (C7′), 115.1 (2C, C3″, C5″), 115.3 (C5′), 124.2 (C8′a), 124.7 (C4), 126.9 (C8′), 128.4 (C3a), 128.6 (C6), 129.2 (2C, C2″, C6″), 131.9 (C1″), 136.5 (C5), 137.4 (C4′a), 141.3 (C7a), 156.5 (C6′), 163.2 (C4″), 178.8 (C2). FTMS+cESI: m/z 409.11 [M+1]⁺

### Example 16

Synthesis of 5-Chloro-1-(4-chlorobenzyl)-8′-hydroxy-3′,4′-dihydro-2′H-spiro[indoline-3,1′-isoquinolin]-2-one (1l) and 5-Chloro-1-(4-chlorobenzyl)-6′-hydroxy-3′,4′-dihydro-2′H-spiro[indoline-3,1′-isoquinolin]-2-one (2l).

Method C. Prepared from 5-chloro-1-(4-chlorobenzyl)indoline-2,3-dione, 8i, (2.4 g, 9.5 mmol) and 3-hydroxyphenethylamine (1.3 g, 5.0 mmol). The reaction products 1l and 2l were separated by column chromatography (hexane: ethyl acetate—80:20).

Yield, 1.1 g, 35% (brown solid). M.p. 122-124° C. ¹H NMR (CD₃OD, 600 MHz): δppm 2.97 (m, H4′a), 3.25 (m, 1H, H4′b), 3.36 (dt, J=13.2, 5.3 Hz, 2H, H3′a, H3′b), 4.80 (d, J=16.1 Hz, 1.11, —CH₂—), 5.15 (d, J=16.1 Hz, 1H, CH₂—Ar), 6.56 (dd, J=8.0, 1.1 Hz, 1H, H7′), 6.74 (d, J=8.4 Hz, 1H, H7), 6.77 (dd, J=7.6, 1.1 Hz, 1H, H5′), 7.05 (d, J=2.12 Hz, 1H, H4), 7.10 (t, J=7.8 Hz, 1H, H6′), 7.18 (dd, J=8.4, 2.1 Hz, 1H, H6), 7.35-7.37 (d, J=8.5 Hz, 2H, H3″), 7.45-7.47(m, 2H, H2″). ¹³C NMR (CD₃OD, 150 MHz): δppm 28.3 (C4′), 38.7 (C3′), 42.9 (—CH₂—Ar), 62.3 (C3/C1′), 110.0 (C7), 112.3 (C7′), 120.1 (C5′), 123.4 (C4), 127.4 (C8′a), 127.8 (C6), 128.2 (C6′), 128.3 (2C, C3″), 128.7 (C2″), 132.9 (C4″), 134.7 (C1″), 135.7 (C5), 138.2 (C4′a), 141.8 (C7a), 154.0 (C8′) 178.6 (C2). FTMS+cESI: m/z 425.08 [M+1]⁺

2l. Yield, 2.0 g, 62% (brown solid). M.p. 208-210° C. ¹C NMR (CD₃OD, 600 MHz): δppm 2.86 (dt, J=16.4, 4.4, 1H, H4′a), 3.01-3.07 (m, 1H, H4′b), 3.18 (ddd, J=12.7, 5.5, 4.2 Hz, 1H, H3′a), 3.85 (ddd, J=12.7, 9.4, 4.2 Hz 1H, H3′b), 4.98 (d, J=15.8 Hz, 1H, —CH₂—Ar), 4.98 (d, J=15.8 Hz, 1H, CH₂—Ar), 6.25 (d, J=8.5 Hz, 1H, H8′), 6.49 (dd, J=8.5, 2.6 Hz, 1H, H7′), 6.67 (d, J=2.6, 1H, H5′), 6.92 (d, J=8.5. 1H, H7), 7.19 (d, J=2.1 Hz, 1H, H4), 7.28 (dd, J=8.4, 2.3 Hz, 1H, H6), 7.36-7.40 (m, 4H, H2″, H3″, H5″, H6″). ¹³C NMR (CD₃OD, 150 MHz): δppm 28.2 (C4′), 38.2 (C3′), 42.5 (—CH₂—Ar), 63.3 (C3/C1′), 110.4 (C7), 113.8 (C7′), 115.3 (C5′), 124.2 (C8′a), 124.7 (C4), 126.9 (C8′), 128.5 (C3a), 128.6 (C6), 128.7 (2C, C2″, C6″), 128.8 (2C, C3″, C5″), 133.2 (C4″), 134.6 (C1″), 136.5 (C5), 137.4 (C4′a), 141.3 (C7a), 156.6 (C6′), 178.8 (C2). FTMS+cESI: m/z 425.08 [M+1]⁺

### Example 17

Synthesis of 5-Chloro-6′-hydroxy-1-(4-methylbenzyl)-3′,4′-dihydro-2′ spiro[indoline-3,1′-isoquinolin]-2-one (2m).

Method C. Prepared from 5-chloro-1-(4-methylbenzyl)indoline-2,3-dione (2 g, 7.0 mmol, 1 equiv) and 3-hyroxyphenethylamine (1.4 g, 10. mmol, 1.5 equiv) in the presence of cataytic amount of TEA. The reaction afforded compounds SE/UB-030/108A and SE/UB-030/108B. The two isomers were separated by column chromatography (hexane: ethyl acetate—80:20).

2o. Yield, 0.7 g, 25% (brown solid). Mp103-105° C. ¹H NMR (CD₃OD, 600 MHz): δppm 2.33 (s, 3H, Ar—CH₃), 2.86 (dt, J=16.4, 4.4, 1H, H4′a), 3.03 (ddd, 1(ddd, J=15.3, 9.2, 5.5 Hz 1H, H4′b), 3.15-3.20 (m, 1H, H3′a), 3.84 (ddd, J=12.8, 9.2, 4.5 Hz 1H, H3′b), 4.79 (d, J=15.6 Hz, 1H, —CH₂—Ar), 4.99 (d, J=15.6 Hz, 1H, CH₂—Ar), 6.24 (d, J=8.5 Hz, 1H, H8′), 6.48 (dd, J=8.5, 2.6 Hz, 1H, H7′), 6.66 (d, J=2.6, 1H, H5′), 6.90 (d, J=8.5, 1H, H7), 7.16-7.19 (m, 3H, H4, H3″; H5″), 7.23-7.28 (m, 3H, H6, H2″, H6″). ¹³C NMR (CD₃OD, 150 MHz): δppm 19.8 (Ar—CH₃), 28.3 (C4′), 38.3 (C3′), 42.9 (—CH₂—Ar), 63.4 (C3/C1′), 110.6 (C7), 113.8 (C7′), 115.1 (C5′), 124.3 (C8′a), 124.6 (C4), 127.0 (C8′), 127.2 (2C, C2″, C6″), 128.3 (C3a), 128.6 (C6), 129.1 (2C, C3″, C5″), 132.7 (C1″), 136.5 (C5), 137.4 (C4″), 137.4 (C4′a), 141.5 (C7a), 156.5 (C6′), 178.8 (C2). FTMS+cESI: m/z 405.14 [M+1]⁺

### Example 18

General Method for the Synthesis of 6′,7′-dihydroxy-3′,4′-dihydro-2′H-spiro[indoline-3,1′-isoquinolin]-2-ones (3,b). Method D.

Dopamine hydrochloride (1 equiv) and the appropriate isatin (1 equiv) were dissolved in ethanol (10 mL) and triethylamine (1 mL) added. The reaction mixture was allowed to stir for 72 hr at room temperature. At the end of the reaction, the solvent was removed in vacuo and distilled water added to the viscous mass. The precipitate obtained was filtered, washed repeatedly with water and dried. The yields from this reaction were over 98%.

### Example 19

6′,7′-Dihydroxy-3′,4′-dihydro-2′H-spiro[indoline-3,1′-isoquinolin]-2-one (3a).

Method D. From isatin (0.4 g, 2.6 mmol) and dopamine (0.5 g, 2.6 mmol). Yield, 0.35 g, 50% (back solid). M.p. 235-237° C.

¹H NMR (CD₃OD, 600 MHz): δppm 2.79 (dt, J=16.2, 4.9 Hz, 1H, H4′a), 2.93 (ddd, J=16.2, 8.6, 5.3 Hz, 1H, H4′b), 3.19-3.23 (m, 1H, H3′a), 3.78 (ddd, J=13.1, 8.6, 4.8 Hz, 1H, H3′b), 5.98 (s, 1H, H8′), 6.61 (s, 1H, H5′), 7.00 (d, J=7.8 Hz, H7), 7.03 (td, J=7.6, 1.1 Hz, 1H, H5), 7.18 (dd, J=7.6, 1.2 Hz, 1H, H4), 7.29 (td, J=7.7, 1.3 Hz, 1H, H6). ¹³C NMR (CD₃OD, 150 MHz): δppm 27.1 (C4′), 38.5 (C3′), 63,6 (C3/C1′), 109.8 (C7), 112.2 (C8′), 115.2 (C5″), 122.7 (C5), 124.2 (C8′a), 124.6 (C4), 126.9 (C4′a), 129.0 (C6), 134.8 (C3a), 141.8 (C7a), 143.8 (C7′), 144.8 (C6′), 180.9 (C2). FTMS+cESI: m/z 283.11 [M+1]⁺

### Example 20

5-Chloro-6′,7′-dibydroxy-3′,4′-dihydro-2′H-spiro[indoline-3,1′-isoquinolin]-2-one (3b).

Method D. Prepared from 5-chloroisatin (0.5 g, 2.6 mmol) and dopamine (0.5 g, 2.6 mmol). Yield, 0.82 g, 98% (black solid). M.p. 210-213° C.

¹H NMR (CD₃OD, 600 MHz): δppm 2.76 (dt, J=16.1, 4.7 Hz, 1H, H4′a), 2.92 (ddd, J=16.2, 8.9, 5.4 Hz, 1H, H4′b), 3.15 (dd, J=12.7, 5.1 Hz, 1H, H3′a), 3.76 (ddd, J=13.1, 8.9, 4.7 Hz, 1H, H3′b), 5.97 (s, 1H, H8′), 6.62 (s, 1H, H5′), 6.97 (d, J=8.3 Hz, H7), 7.17 (dd, J=1.2 Hz, 1H, H4), 7.29 (dd, J=8.3, 2.2 Hz, 1H, H6). ¹³C NMR (CD₃OD, 150 MHz): δppm 29.2 (C4′), 38.5 (C3′), 63.8 (C3/C1′), 110.9 (C7), 112.1 (C8′), 115.3 (C5′), 123.8 (C8′a), 124.9 (C4), 127.1 (C4′a), 127.1 (C5), 128.7 (C6), 137.0 (C3a), 140.6 (C7a), 143.8 (C7′), 145.0 (C6′), 180.6 (C2). FTMS+cESI: m/z 317.07 [M+1]⁺

### Example 21

5,7-Dibromo-6′,7′-dihydroxy-3′,4′-dihydro-2′H-spiro[indoline-3,1′-isoquinolin]-2-one (3c).

Method D. Prepared from 5,7-dibromoisatin (1.56 g, 5.1 mmol), dopamine (1 g, 5.1 mmol). Yield, 1.7 g, 76% (brown solid). M.p. 256-258° C. (HCl salt).

¹H NMR (CD₃OD, 700 MHz): δppm 2.74 (di, J=16.1, 4.6 Hz, 1H, H4′a), 2.90 (ddd, J=15.9, 5.4 Hz, 1H, H4′b), 3.10-3.15 (m, H3′a), 3.71-3.77 (m, H3′b), 5.91 (s, 1H, H8′), 6.62 (s, 1H, H5′), 7.27 (d, J=1.8 Hz, 1H, H4), 7.64 (d, J=1.8 Hz, 1H, H6). ¹³C NMR (CD₃OD, 175 MHz): δppm 27.2 (C4′), 38.4 (C3′), 64.7 (C3/C1′), 102.8 (C7), 111.9 (C8′), 114.9 (C5), 115.4 (C5′), 123.5 (C 8′a), 126.7 (C4), 127.3 (C4′a), 133.6 (C6), 138.7 (C3a), 140.9 (C7a), 143.8 (C7′), 145.0 (C6′), 180.1 (C2). FTMS+cESI: m/z 440.92 M^{+.}

### Example 22

Synthesis of Substituted Beta-Nitrostyrenes (10a,b). (Method E)

The method of Maresh et al. (2014) was employed here with some modification. A solution of substituted benzaldehyde (1 equiv), nitromethane (2 equiv), and anhydrous ammonium acetate (0.1 equiv) in acetic acid was refluxed for six hours. The reaction mixture was then diluted slowly while stirring, with 200 mL water. During this time, a heavy yellow crystalline mass was formed. This was removed by filtration, washed with water, and sucked as dry as possible, recrystallized from boiling methanol and air dried, yielding the β-nitrostyrene as bright yellow crystals in over 95% yield. The compound was used without further purification.

### Example 23

Synthesis of Methoxyphenethylamines (11a,b). Method F

This reaction carried out following the method of Maresh et al. (2014). Typically, 1.0 mmol of nitrostyrene required 2 mL of methanol, 800 mg of zinc dust (12 mmol), and 2 mL of 37% HCl (24 mmol). Methanol was stirred vigorously in an ice bath maintained <0° C. (ice/NaCl). Conc. HCl, zinc dust, and the appropriate nitrostyrene were slowly added over the course of 30 minutes in small portions while ensuring that the temperature did not rise above 0° C. After complete addition of all starting materials, any solids on the side were washed into the solution with a small amount of methanol. The reaction mixture was allowed to further stir for 1 hour until the disappearance of the initial yellow colour of the nitrostyrene was observed. The reaction was further stirred for 4-6 hours after the yellow colour had disappeared. Thereafter, the reaction mixture was placed in a 4° C. refrigerator overnight. Upon completion of the reaction, the excess solid zinc was removed by filtration through filter paper. The solution was made basic by dropwise addition of saturated sodium hydroxide in methanol, while maintaining the temperature below 5° C., until the pH was greater than 11 as indicated by pH paper. The product was then extracted into dichloromethane and the solution was dried over anhydrous sodium sulphate. The insoluble zinc hydroxide which precipitates out during the addition of sodium hydroxide solution was extracted many more times with dichloromethane and filtered. The combined organic extracts were evaporated in vacuo to yield the corresponding phenethylamine as a yellow viscous oil in about 70% yield.

### Example 24

General method for the synthesis of 6′-methoxy-3′,4′-dihydro-2′H-spiro[indoline-3,1′-isoquinolin]-2-ones(4a-c, 5a-c, 6a-c) and 1-(substitutedbenzyl)-6′-methoxy-3′,4′-dihydro-2′H-spiro[indoline-3,1′-isoquinolin]-2-ones (4d-f, 5d-f, 6d-f). Method G.

A mixture of the appropriate isatin (1 equiv), 3-methoxyphenethylamine (1.2 equiv) and polyphosphoric acid (PPA) (2 g) was heated in an oil bath (bath temperature at 100° C.), while stirring mechanically for 5 hours. Upon completion of the reaction, as revealed by TLC, the reaction mixture was allowed to cool to about 50° C. and quenched by slow addition of water. To this mixture was added a saturated solution of sodium carbonate to make the mixture basic to pH 11. The floating product obtained was extracted into ethyl acetate (3×30 mL). The combined organic extracts were dried over anhydrous sodium sulphate, and concentrated under reduced pressure to obtain the crude product. This crude product was purified by flash chromatography on silica gel using suitable solvent systems. Yields ranged between 70 and 98%.

### Example 25

6′-Methoxy-3′,4′-dihydro-2′H-spiro[indoline-3,1′-isoquinolin]-2-one (4a).

Method G. Prepared frotnisatin (0.81 g, 5.5 mmol), 3-methoxyphenethylamine (1 g, 6.6 mmol) and polyphosphoric acid (PPA) (3 g). The crude product was purified by flash chromatography (hexane: ethyl acetate—60:40), Yield. 1.1 g, 73% (Yellow oil).

¹H NMR (CD₃OD, 600 MHz): δppm 2.91 (dt, J=16.6, 4.8 Hz, 1H, H4′a), 3.05 (ddd, J=16.6, 8.7, 5.4 Hz, 1H, H4′b), 3.20 (dt, J=12.9. 5.2 Hz, 1H, H3′a), 3.76 (m, 4H, OCH₃, H3′b), 6.45 (d, J=8.6 Hz, 1H, H8′), 6.61 (dd, J=8.6, 2.7 Hz, 1H, H7′), 6.77 (d, J=2.7 Hz, 1H, H5′), 6.98-7.03 (m, 2H, H5, H7), 7.12 -7.14 (m, 1H, H4), 7.29 (td, J=7.7, 1.3 Hz, 1H, H6). ¹³C NMR (CD₃OD, 150 MHz): δppm 28.4 (C4′), 38.2 (C3′), 54.2 (OCH₃), 63.7 (C3/C1′), 109.7 (C7), 112.6 (C7′), 113.4 (C5′), 122.6 (C5), 124.4 (C4), 126.1 (C8′a), 127.2 (C8′), 128.9 (C6), 135.2 (C3a), 137.3 (C4′a), 141.8 (C7a), 158.8 (C6′), 181.1 (C2). FTMS+cESI: m/z 281.13 [M+1]⁺

### Example 26

5-Chloro-6′-methoxy-3′,4′-dihydro-2′H-spiro[indoline-3,1′-isoquinolin]-2-one (4b).

Method G. Prepared from 5-chloroisatin (1 g, 5.5 mmol) and 3-methoxyphenethylamine (1 g, 6.6 mmol) in polyphosphoric acid (PPA) (5 g). The crude product was purified by flash chromatography (hexane:ethyl acetate, 50:50). Yield, 0.6 g, 35% (green solid). M.p. 114-116° C.

¹H NMR (CD₃OD, 600 MHz): δppm 2.90 (dt, J=16.6, 4.6 Hz, 1H, H4′a), 3.05 (ddd, J=16.5, 8.9, 5.4 Hz, 1H, H4′b), 3.17 (dt, J=12.7, 5.0 Hz, 1H, H3′a), 3.77 (m, 4H, OCH₃, H3′b), 6.46 (d, J=8.6 Hz, 1H, H8′), 6.65 (dd, J=8.7, 2.7 Hz, 1H, H7′), 6.78 (d, J=2.7 Hz, 1H, H5′), 6.97 (d, J=8.3 Hz, 1H, H7), 7.13 (d, J=2.1 Hz, 1H, H4), 7.30 (dd, J=8.3, 2.2 Hz, 1H, H6), ¹³C NMR (CD₃OD, 150 MHz): δppm 28.4 (C4′), 38.2 (C3′), 54.3 (OCH₃), 63.8 (C3/C1′), 110.9 (C7), 112.7 (C7′), 113.5 (C5′), 124.8 (C4), 125.5 (C8′), 127.0 (C8′), 127.6 (C3a), 128.7 (C6), 137.1 (C5), 137.4 (C4′a), 140.6 (C7a), 159.0 (C6′), 180.7 (C2). FTMS+cESI: m/z 315.09 [M+1]+

### Example 27

5,7-Dibromo-6′-methoxy-3′,4′-dihydro-2′H-spiro[indoline-3,1′-isoquinolin]-2-one (4e).

Method G. Prepared from 5,7-dibromoisatin, 7c,(2.02 g, 6-6 mmol) and 3-ethoxyphenethylamine (1 g, 6.6 mmol) in PPA (3 g). The crude product was purified by flash chromatography (hexane: ethyl acetate—90:10). Yield 2.03 g, 70% (brown solid). M.p. 255-257° C. ¹H NMR (CD₃OD, 600 MHz); δppm 3.52 (dt, J=16.1, 3.9 Hz, 1H, H4′a), 3.73 (ddd, J=15.4, 9.5, 5.5 Hz, 1H, H4′b), 3.79 (ddd, J=12.2, 5.5, 3.6 Hz, 1H, H3′a), 4.40 (m, 1H, H3′b), 4.52 (s, 3H, OCH₃), 7.43 (dd, J=8.6, 2.8 Hz, 1H, H7′), 7.56 (d, J=2.8 Hz, 1H, H5′), 7.94 (d, J=1.8 1H, H4), 7.18 (d, J=8.6 Hz, 1H, H8′), 8.48 (d, J=1.9 Hz, 1H, H6), 11.57 (br. s, 1H, H1). ¹³C NMR (CD₃OD, 150 MHz): δppm 30.1 (C4′), 39.1 (C3′), 56.3 (OCH₃), 65.6 (C3/C1′), 104.1 (C7), 114.1 (C7′), 115.0 (C5′), 115.2 (C5), 127.4 (C8′a), 127.7 (C4), 128.3 (C8′), 134.5 (C6), 139.1 (C4′a), 140.7 (C3a), 142.7 (C7a), 159.3 (C6′), 181.0 (C2). FTMS+cESI: m/z. 438.95 [M+1]⁺.

### Example 28

6′-Methoxy-1-(4-fluorobenzyl)-3′,4′-dihydro-2′H-spiro[indoline-3,1′-isoquinolin]-2-one (4d).

Method G. Prepared from 1-(4-fluorobenzyl)indoline-2,3-dione, 8a,(1.5 g, 6.0 mmol) and 3-methoxyphenethylamine (0.9 g, 6.0 mmol) in polyphosphoric acid (3 g). The crude product was purified by flash chromatography (hexane: ethyl acetate—70:30). Yield, 1.7 g, 73% (Yellow oil). ¹H NMR (CD₃OD, 600 MHz): δppm 2.93 (dt, J=16.5, 4.5 Hz, 1H, H4′a), 3.06-3.11 (m, 1H, H4′b), 3.22 (dt, J=12.8, 5.0 Hz, 1H, H3′a), 3.76 (s, 3H, 6′-OCH₃), 3.87 (ddd, J=13.3, 9.1, 4.6 Hz, 1H, H3′b), 4.90 (d, J=15.6, 1H, —CH₂—Ar), 5.00 (d, J=15.6 Hz, 1H, CH₂—Ar), 6.32 (d, J=8.6 Hz, 1H, H8′), 6.59 (dd, J=8.6, 2.7 Hz, 1H, H7′), 6.79 (d, J=2.7 Hz, 1H, H5′), 6.97 (d, J=8.0 Hz, 1H, H7), 7.05 (td, J=7.5, 0.9 Hz, 1H, H5) 7.07-7.11 (m, 2H, H3″, H5″), 7.18 (dd, J=7.4, 1.2 Hz, 1H, H4), 7.28 (td, J=7.8, 1.3 Hz, 1H, H6), 7.42-7.46 (m, 2H, H2″, H6″). ¹³C NMR (CD₃OD, 150 MHz): δppm 28.4 (C4′), 38.3 (C3′), 42.4 (—CH₂—Ar), 54.1 (OCH₃), 63.3 (C3/C1′), 109.3 (C7), 112.6 (C7′), 113.6 (C5′), 115.0 (2C, C3″, C5″), 123.2 (C5), 124.3 (C4), 126.1 (C8′a), 127.0 (C8′), 128.9 (C6), 129.2 (2C, C2″, C6″), 132.1 (C1″), 134.5 (C3a), 137.5 (C4′a), 142.6 (C7a), 158.9 (C6′), 163.2 (C4″), 179.0 (C2). FTMS+cESI: m/z 389.17 [M+1]⁺

### Example 29

6′-Methoxy-1-(4-methylbenzyl)-3′,4′-dihydro-2′H-spiro[indoline-3,1′-isoquinolin]-2-one (4e).

Method G. Prepared from 8e (1.5 g, 6.0 mmol) and 3-methoxyphenethylamine (0.9 g, 0.9 mL, 6.0 mmol) in PPA (3 g), and purified by flash chromatography (hexane: ethyl acetate—70:30). Yield 2.0 g, 87%. (Yellow oil). ¹H NMR (CD₃OD, 600 MHz): δppm 2.32 (s, 3H, 4″—CH₃-Bz), 2.94 (dt, J=16.5, 4.6 Hz, 1H, H4′a), 3.06-3.12 (m, 1H, H4′b), 3.21-3.26 (m, 1H, H3′a), 3.75 (s, 3H, 6′-OCH₃), 3.88 (ddd, J=13.3, 9.0, 4.7 Hz, 1H, H3′b), 4.81 (d, J=15.5, 1H, —CH₂—Ar), 5.00 (d, J=15.5 Hz, 1H, CH₂—Ar), 6.34 (d, J=8.7 Hz, 1H, H8′), 6.58 (dd, J=8.6, 2.7 Hz, 1H, H7′), 6.79 (d, J=2.7 Hz, 1H, H5′), 6.95 (d, J=7.9 Hz, 1H, H7), 7.03 (td, J=7.5, 1.0 Hz, 1H, H5) 7.15-7.18 (m, 3H, H4, H3″, H5″), 7.25 (td, J=7.7, 1.3 Hz, 1H, H6), 7.28 (d, J=7.9 Hz, 2H, H2″, H6″). ¹³C NMR (CD₃OD, 150 MHz): δppm 19.8 (4″-CH₃), 28.3 (C4′), 38.3 (C3′), 42.9 (—CH₂—), 54.3 (OCH₃), 63.3 (C3/C1′), 109.5 (C7), 112.7 (C7′), 113.6 (C5′), 123.2 (C5), 124.2 (C4), 126.0 (C8′a), 127.1 (C8′), 127.2 (2C, C2″, C6″), 128.9 (C6), 129.1 (2C, C3″, C5″), 133.0 (C1″), 134.2 (C3a), 137.2 (C4″), 137.3 (C4′a), 142.7 (C7a), 158.9 (C6′), 178.8 (C2). FTMS+cESI: m/z 385.19 [M+1]⁺

### Example 30

6′-Methoxy-1-phenyl-3′,4′-dihydro-2′H-spiro[indoline-3,1′-isoquinolin]-2-one (4g).

Method G. Prepared from 1-phenylisatin (0.5 g, 2.2 mmol), 3-methoxyphenethylamine (0.4 g, 2.7 mmol) and polyphosphoric acid (2 g). The crude product was purified by flash chromatography (hexane:ethyl acetate—80:20). Yield, 0.63 g, 79% (brown solid), M.p. 113-115° C.

¹H NMR (DMSO-d₆, 700 MHz): δppm 2.78 (dt, J=16.05, 3.72 Hz, 1H, H4′a), 2.96-3.02 (m, 1H, H4′b), 3.05 (dt, J=9.3, 3.1 Hz, 1H, H3′a), 3.69-3.75 (m, 4H, OCH₃, H3′b), 6.50 (d, J=8.6 Hz, 1H, H8′), 6.63 (dd, J=8.6, 2.7 Hz, 1H, H7′), 6.77 (d, J=2.7 Hz, 1H, H5′), 6.78 (dt, J=7.85, 0.7 Hz, 1H, H7), 7.04 (d, J=7.5, 1.0 Hz, 1H, H5), 7.14 (dd, J=7.4, 1.3 Hz, 1H, H4), 7.26 (td, J=7.7, 1.3 Hz, 1H, H6), 7.46 (dd, J=8.4, 7.2, Hz, 3H, H3″, H4″, H5″), 7.56-7.60 (m, 2H, H2″, H6″). ¹³C NMR (DMSO-d₆, 175 MHz): δppm 29.5 (C4′), 38.5 (C3′), 55.5 (6′-OCH₃), 63.4 (C3/C1′), 109.3 (C7), 113.2 (C7′), 114.1 (C5′), 123.6 (C5), 125.3 (C4), 127.3 (C3″, C5″), 127.7 (C4″), 127.7 (C8′), 128.4 (C8′a), 129.2 (C6), 130.1 (C2″, C6″), 134.9 (C1″), 135.5 (C3a), 138.4 (C4′a), 144.0 (C7a), 158.4 (C6′), 178.3 (C2). FTMS+cESI: m/z 357.16 [M+1]⁺.

### Example 31

6′-Methoxy-5-methyl-3′,4′-dihydro-2′H-spiro[indoline-3,1′-isoquinolin]-2-one (4i).

Method G. Prepared from 5-methylisatin (2.8 g, 17 mmol), 3,4-dimethoxyphenethylamine (2.6 g 17 mmol) and polyphosphoric acid (3 g). The crude product was purified by flash chromatography (hexane:ethyl acetate—80:20). Yield, 4.6 g, 92% (brown solid), M.p. 208-209° C.

¹H NMR (DMSO-d₆, 600 MHz): δppm 1.46 (s, 3H, 5—CH₃), 2.07-2.13 (m, 1H, H4′a), 2.23 (ddd,J=16.5, 8.7, 5.3 Hz, 1H, H4′b), 2.39 (dt, J=12.8. 5.2 Hz, 1H, H3′a), 2.96 (d, J=5.1 Hz, 4H, H3′b, m, 4H, 7′-OCH₃), 5.64 (d, J=8.6 Hz, 1H, H8′), 5.80 (dd, J=8.6, 2.7 Hz, 1H, H7′), 5.95 (d, J=2.7 Hz, 1H, H5′), 6.07 (d, J=7.87 Hz, 1H, H7), 6.14-6.17 (m, 1H, H4), 6.29 (ddd, J=7.9, 1.7, 0.8 Hz, 1H, H6). ¹³C NMR (DMSO-d₆, 150 MHz): δppm 18.9 (5-CH₃), 27.6 (C4′), 37.5 (C3′), 53.4 (6′-OCH₃), 62.9 (C3/C1′), 108.7 (C7), 111.8 (C7′), 112.6 (C5′), 124.3 (C4), 125.4 (C8′a), 126.4 (C8′), 128.3 (C6), 131.5 (C3a), 134.5 (C7a), 136.4 (C4′a), 138.4 (C5), 158.0 (C6′), 180.3 (C2). FTMS+cESI: m/z 295.14 [M+1]⁺.

### Example 32

6′,7′-Dimethoxy-3′,4′-dihydro-2′H-spiro[indoline-3,1′-isoquinolin]-2-one (5a).

Method G. Prepared from isatin (1.0 g, 6.8 mmol) and 3,4-dimethoxyphenethylamine, 11a, (1 g, 6.6 mmol)] in PPA (3 g). The crude product was purified by flash chromatography (hexane: ethyl acetate—70:30) Yield 2.07 g, 99% (yellow solid). M.p. 188-191° C.

¹H NMR (CD₃OD, 600 MHz): δppm 2.86 (dt, J=16.3, 4.9 Hz, 1H, H4′a), 2.95-3.03 (m, 1H, H4′b), 3.21 (dt, J=12.9, 5.2 Hz, 1H, H3′a), 3.52 (s, 3H, 7′-OCH₃, H3′b), 3.76 (ddd, J=13.1, 8.5, 4.8 Hz, 1H, H3′b), 3.83 (s, 3H, 6-OCH₃), 6.03 (s, 1H, H8′), 6.79 (s, 1H, H5′), 7.01 (dt, J=7.8, 0.8 Hz, H7), 7.03 (td, J=7.6, 1.1 Hz, 1H, H5), 7.15 (ddd, J=7.5, 1.3, 0.6 Hz, 1H, H4), 7.30 (td, J=7.7, 1.3 Hz, 1H, H6). ¹³C NMR (CD₃OD, 150 MHz): δppm 27.6 (C4′), 38.4 (C3′), 54.9 (7′-OCH₃), 55.0 (6′-OCH₃), 63.8 (C3/C1′), 109.2 (C8′), 109.8 (C7), 112.2 (C5′), 122.6 (C5), 124.5 (C4), 125.8 (C8′a), 128.9 (C4′a), 129.0 (C6), 135.0 (C3a), 141.7 (C7a), 147.7 (C7′), 148.0 (C6′), 180.9 (C2). FTMS+cESI: m/z 311.14 [M+1]⁺

### Example 33

5-Chloro-6′,7′-dimethoxy-3′,4′-dihydro-2′H-spiro[indoline-3,1′-isoquinolin]-2-one (5b).

Method G. From 5-chloroisatin (1.5 g, 8.3 mmol), 3,4-dimethoxyphenethylamine, 11a, (1.5 g, 8.3 mmol) in polyphosphoric acid (3 g). The crude product was purified by flash chromatography (hexane: ethyl acetate—70:30); Yield 1.8 g, 63% (white solid). M.p. 138-140° C.

¹H NMR (CD₃OD, 600 MHz): δppm 2.83-288 (m, 1H, H4′a), 2.96-3.02 (m, 1H, H4′b), 3.18 (dt, J=12.7, 5.1 Hz, 1H, H3′a), 3.56 (s, 3H, 7′-OCH₃), 3.75 (ddd, J=13.1, 8.6, 4.7 Hz, 1H, H3′b), 3.84 (s, 6′-OCH₃), 6.03 (s, 1H, H8′), 6.81 (s, 1H, H5′), 6.99 (dt, J=8.3, 1H, H7), 7.16 (d, J=2.1 Hz, 1H, H4), 7.30-7.32 (m, 1H, H6). ¹³C NMR (CD₃OD 150 MHz): δppm 27.5 (C4′), 38.3 (C3′), 55.0 (7′-OCH₃), 55.1 (6′-OCH₃), 64.0 (C3/C1′), 109.0 (C8′), 111.0 (C7), 112.3 (C5′), 124.8 (C4), 125.1 (C8′a), 127.7 (C3a), 128.9 (C6), 129.0 (C4′a), 136.8 (C5), 140.6 (C7a), 147.8 (C7′), 148.9 (C6′), 180.6 (C2). FTMS+cESI: m/z 345.10 [M+1]⁺

### Example 34

5,7-dibromo-6′,7′-dimethoxy-3′,4′-dihydro-2′H-spiro[indoline-3,1′-isoquinolin]-2-one (5c).

Method G. Prepared from 5,7-dibromoisatin, 7c, (1.0 g, 3.3 mmol), 3,4-dimethoxyphenethylamine, 11a, (0.6 g, 3.3 mmol) and polyphosphoric acid (3 g). The crude product was purified by flash chromatography (hexane: ethyl acetate—70:30). Yield, 1.8 g, 63% (white solid). M.p. 180-183° C.

¹H NMR (CD₃OD D, 600 MHz): δppm 2.84 (dt, J=16.3, 4.7, 1H, H4′a), 2.96-3.03 (m, 1H, H4′b), 3.16 (dt, J=12.8, 5.1 Hz, 1H, H3′a), 3.58 (s, 3H, 7′-OCH₃), 3.73 (ddd, J=13.1, 8.7, 4.7 Hz, 1H, H3′b), 3.84 (s, 3H, 6′-OCH₃), 6.04 (s, 1H, H8′), 6.81 (s, 1H, H5′), 7.26 (d, J=1.8 Hz, 1H, H4), 7.66 (d, J=1.8 1H, H6). ¹³C NMR (CD₃OD, 150 MHz): δppm 27.5 (C4′), 38.2 (C3′), 55.0 (7′-OCH₃), 55.2 (6′-OCH₃), 64.9 (C3/C1′), 103.0 (C7), 109.0 (C8′), 112.4 (C5′), 114.9 (C5), 124.6 (C8′a), 126.7 (C4), 129.1 (C4′a), 133.9 (C6), 138.2 (C5), 141.0 (C7a), 147.9 (C7′), 149.1 (C6′), 179.7 (C2). FTMS+cESI: m/z 468.96 [M+1]⁺

### Example 35

1-(4-fluorobenzyl)-6′,7′-dimethoxy-3′,4′-dihydro-2′H-spiro[indoline-3,1′-isoquinolin]-2-one (5d).

Method G. Prepared from 1-(4-fluorobenzyl)indoline-2,3-dione, 8a, (2.0 g, 7.8 mmol) and 3,4-dimethoxyphenethylamine, 11a, (1.4 g, 7.8 mmol) in polyphosphoric acid (2.5 g). The crude product was purified by flash chromatography (hexane: ethyl acetate—90:10). Yield, 2.2 g, 88% (brown solid). M.p. 145-149° C. ¹H NMR (CD₃OD, 600 MHz): δppm 2.88 (dt, J=16.2, 4.6 Hz, 1H, H4′a), 2.99-3.06 (m, 1H, H4′b), 3.21-3.26 (m, 1H, H3′a), 3.38 (s, 3H, 7′-OCH₃), 3.82 (s, 4H, H3′b, 6′-OCH₃), 4.82 (d, J=15.5 Hz, 1H, —CH₂—Ar), 5.09 (d, J=15.5 Hz, 1H, CH₂—Ar), 5.82 (s, 1H, H8′), 6.80 (s, 1H, H5′), 7.04-7.11 (m, 4H, H5, H7, H3″, H5″), 7.20 (dd, J=7.5, 1.2 Hz, 1H, H4), 7.31 (td, J=7.8, 1.3 Hz, 1H, H6), 7.45-7.48 (m, 2H, H2″, H6″). ¹³C NMR (CD₃OD, 150 MHz): δppm 27.6 (C4′), 38.5 (C3′), 42.3 (—CH₂—Ar), 54.8 (7′-OCH₃), 55.0 (6′-OCH₃), 63.3 (C3/C1′), 108.8 (C8′), 109.3 (C7), 112.2 (C5′), 115.2 (2C, C3″, C5″), 123.2 (C5), 124.3 (C4), 125.8 (C8′a), 128.9 (C6), 129.4 (2C, C2″,C6″), 132.4 (C1″), 134.3 (C3a), 137.4 (C4″), 142.4 (C7a), 147.7 (C7′), 148.8 (C6′), 163.2 (C4″), 178.8 (C2). FTMS+cESI: m/z 419.18 [M+1]⁺

### Example 36

6′,7′-Dimethoxy-1-(4-methylbenzyl)-3′,4′-dihydro-2′H-spiro[indoline-3,1′-isoquinolin]-2-one (5e).

Method G. Prepared from 1-(4-methylbenzyl)indoline-2,3-dione, 8e, (1.5 g. 6.0 mmol) and 3,4-dimethoxyphenethylamine, 11a, (1 g 6.0 mmol) in polyphosphoric acid (2.5 g). Purified by flash chromatography (hexane: ethyl acetate—90:10). Yield, 2.2 g, 88% (brown solid). M.p. 175-177° C. ¹H NMR (CD₃OD, 600 MHz): δppm 2.32 (s, 3H, 4″—CH₃-Bz), 2.89 (dt, J=16.2, 4.7 Hz, 1H, H4′a), 3.03 (ddd, J=16.2, 8.7, 5.3 Hz, 1H, H4′b), 3.24 (dt, J=12.8, 5.1 Hz, 1H, H3′a), 3.37 (s, 3H, 7′-OCH₃), 3.83 (s, 4H, H3′b, 6′-OCH₃), 4.75 (d, J=15.4 Hz, 1H, —CH₂—Ar), 5.13 (d, J=15.4 Hz, 1H, CH₂—Ar), 5.82 (s, 1H, H8′), 6.81 (s, 1H, H5′), 7.04 (d, J=7.9 Hz, 1H, H7), 7.06 (td, J=7.6, 1.0 Hz, 1H, H5), 7.17 (d, J=7.9 Hz, 2H, H3″, H5″), 7.20 (dd, J=7.5, 1.2 Hz, 1H, H4), 7.30 (td, J=7.8, 1.3 Hz, 1H, H6), 7.32 (m, 2H, H2″, H6″). ¹³C NMR (CD₃OD, 150 MHz): δppm 19.7 (4″-CH₃), 27.5 (C4′), 38.5 (C3′), 42.8 (—CH₂—Ar), 54.7 (7′-OCH₃), 55.0 (6′-OCH₃), 63.4 (C3/C1′), 108.8 (C8′), 109.5 (C7), 112.2 (C5′), 123.2 (C5), 124.2 (C4), 125.8 (C8′a), 127.4 (2C, C2″, C6″), 128.8 (C4′a), 128.9 (C6), 129.0 (2C, C3″, C5″), 133.2 (C1″), 134.3 (C3a), 137.4 (C4″) 142.6 (C7a), 147.7 (C7′), 148.7 (C6′), 178.9 (C2). FTMS+cESI: m/z 415.20 [M+1]⁺

### Example 37

1-(4-Bromobenzyl)-5-chloro-6′,7′-dimethoxy-3′,4′-dihydro-2′H-spiro[indoline-3,1′-isoquinolin]-2- one (5f).

Method G. Prepared from 1-(4-bromobenzyl)-5-chloroindoline-2,3-dione, 8c, (0.7 g, 4.1 mmol) and 3,4-dimethoxyphenethylamine, 11a, (1.2 g 3.4 mmol) in polyphosphoric acid (2.0 g). Product purified by flash chromatography (hexane: ethyl acetate—(80:20). Yield, 1.2 g, 69% (brown solid). M.p. 198-201° C. ¹H NMR (CD₃OD, 600 MHz): δppm 2.84-2.90 (m, 1H, H4′a), 3.02 (ddd, J=9.7, 5.3, 4.5 Hz, 1H, H4′b), 3.18-3.23 (m, 1H, H3′a), 3.42 (s, 3H, 7′-OCH₃), 3.82 (d, J=5.9 Hz, 1H, H3′b), 3.84 (s, 3H, 6′-OCH₃), 4.83 (d, J=15.7 Hz, 1H, —CH₂—Ar), 5.06 (d, J=15.7 Hz, 1H, CH₂—Ar), 5.80 (s, 1H, H8′), 6.82 (s, 1H, H5′), 7.04 (d, J=8.4 Hz, 1H, H7), 7.21 (d, J=72.2 Hz, 1H, H4), 7.33 (dd, J=8.4, 2.2 Hz, 1H, H6), 7.37 (d, J=8.4, 2H, H2″, H6″), 7.50-7.54 (m, 2H, H3″, H5″). ¹³C NMR (CD₃OD, 150 MHz): δppm 27.5 (C4′), 38.4 (C3′), 42.5 (—CH₂—Ar), 54.9 (7′-OCH₃), 55.0 (6′-OCH₃), 63.4 (C3/C1′), 108.6 (C8′), 110.5 (C7), 112.4 (C5′), 121.4 (C4″), 124.7 (C4), 125.0 (C8′a), 127.8 (C4′a), 128.5 (C6), 129.0 (C5), 129.4 (2C, C2″, C6″), 131.6 (2C, C3″, C5″), 135.3 (C1″), 136.2 (C3a), 141.1 (C7a), 147.8 (C7′), 149.0 (C6′), 178.5 (C2). FTMS+cESI: m/z 513.06 [M+1]⁺

### Example 38

6′,7′-Dimethoxy-1-phenyl-3′,4′-dihydro2′H-spiro[indoline3,1′-isoquinolin]-2-one (5g).

Method G. Prepared from 1-phenylisatin (0.5 g, 2.2 mmol), 3,4-dimethoxyphenethylamine (0.5 g, 2.6 mmol) and polyphosphoric acid (2 g). The crude product was purified by flash chromatography (hexane:ethyl acetate—60:40). Yield, 0.72 g, 85% (brown solid), M.p. 227-229° C. (HCl salt).

¹H NMR (DMSO-d₆, 600 MHz): δppm 1.94 (ddd, J=16.4, 5.8. 3.6 Hz, 1H, H1′), 2.13 (ddd, J=15.8, 9.1, 5.8 Hz, 1H, H4′a), 2.31 (dt, J=12.6, 4.6 Hz, 1H, H3′a), 2.62 (m, 3H, 7′-OCH₃), 2.88-2.96 (m, 5H, H3′b, H4′b, 6′-OCH₃), 5.21 (d, J=2.5 Hz, 1H, H8′), 5.90 (d, J=2.5 Hz, 1H, H5′), 5.95 (dd, J=7.9, 2.6 Hz, 1H, H7), 6.21 (td, J=7.6, 2.7 Hz, 1H, H5), 6.35 (dd, J=7.6, 2.8 Hz, 1H, H4), 6.37-6.41 (m, 1H, H4″), 6.54-6.59 (m, 3H, H6, H2″, H6″), 6.65-6.69 (m, 2H, H3″, H5″). ¹³C NMR (DMSO-d₆, 150 MHz): δppm 26.8 (C4′), 37.6 (C3′), 54.2 (7′-OCH₃), 54.3 (6′-OCH₃), 62.6 (C3/C1′), 108.4 (C8′), 108.5 (C7), 111.5 (C5′), 122.9 (C5), 123.9 (C4), 124.9 (C8′a), 125.8 (C2″, C6″), 127.4 (C3a), 128.3 (C4″), 128.3 (C4′a), 128.7 (C3″, C5″), 133.1 (C6), 133.5 (C1″), 143.0 (C7a), 147.0 (C7′) 148.1 (C6′), 177.3 (C2). FTMS+cESI: m/z 387.17 [M+1]⁺.

### Example 39

5,7-Dibromo-6′,7′-dimethoxy-1-(4-methylbenzyl)-3′,4′-dihydro-2′H-spiro[indoline-3,1′-isoquinolin]-2-one (5h).

Method G. Prepared from 5,7-dibromo-1-(4-chlorobenzyl)indoline-2,3-dione (1.8 g, 4.4 mmol), 3,4-dimethoxyphenethylamine (0.8 g, 4.4 mmol) and polyphosphoric acid (2 g). The crude product was purified by flash chromatography (hexane: ethyl acetate—60:40). Yield, 1.8 g, 71% (yellow oil), M.p. 239-240° C. (HCl salt)

1H NMR (DMSO-d₆, 600 MHz): δppm 1.93 (dt, J=16.2, 4.3 Hz, 1H, H4′a), 2.11 (ddt, J=15.3, 9.3, 4.9 Hz, 1H, H4′b), 2.27 (ddd, J=12.7, 5.4, 4.0 Hz, 1H, H3′a), 2.58 (s, 3H, 7′-OCH₃), 2.85-2.89 (m, 1H, H3′b), 2.92 (s, 3H, 6′-OCH₃), 4.39 (d, J=16.1 Hz, 1H, CH₂—Ar) 4.51 (d, J=16.3 Hz, 1H, CH₂—Ar), 5.00 (s, 1H, H8′), 5.91 (s, 1H, H5′), 6.23 (d, J=7.8 Hz, 2H, H3″, H5″), 6.30 (m, 2H, H2″, H6″), 6.40 (d, J=2.0 Hz, 1H, H4), 6.77 (d, J=1.9 Hz, 1H, H6). ¹³C NMR (DMSO-d₆, 150 MHz): δppm 18.8 (4″—CH₃), 28.6 (C4′), 37.5 (C3′), 43.0 (CH₂—Ar), 54.2 (7′-OCH₃), 54.2 (6′OCH₃), 62.0 (C3/C1′), 101.9 (C7), 107.9 (C8′), 111.6 (C5′), 115.0 (C5), 123.8 (C8′a), 125.5 (C2″, C6″), 126.3 (C4), 128.1 (C3″, C5″), 128.3 (C4′a), 133.5 (C1″), 135.8 (C6), 136.0 (C3a), 138.5 (C4″), 139.0 (C7a), 147.1 (C7′), 148.3 (C6′), 178.3 (C2), FTMS+cESI: m/z 573.02 [M+1]⁺.

### Example 40

1-(4-Fluorobenzyl)-6′,7′-dimethoxy-5-methyl-3′,4′-dihydro-2′H-spiro[indoline-3,1′-isoquinolin]-2-one (5j).

Method G. Prepared from 5-methyl-1-(4-fluorobenzyl)indoline-2,3-dione (1 g, 3.7 mmol), 3,4-dimethoxyphenethylamine (0.8 g, 4.4 mmol) and polyphosphoric acid (3 g). The crude product was purified by flash chromatography (hexane: ethyl acetate—60:40). Yield, 1.4 g, 90% (brown solid), M.p. 99-101° C.

¹H NMR (DMSO-d₆, 600 MHz): δppm 2.19 (s, 3H, 5-CH₃), 2.71 (dt, J=15.9, 4.1 Hz, 1H, H4′a), 2.88 (ddd, J=15.1, 9.3, 5.4 Hz, 1H, H4′b), 3.05 (ddd, J=12.5, 5.4, 4.1 Hz, 1H, H3′a), 3.29 (s, 3H, 7′-OCH₃), 3.65 (ddd, J=12.4, 9.3, 4.3 Hz, 1H, H3′b), 3.74 (s, 3H, 6′-OCH₃), 4.76 (d, J=15.6 Hz, 1H, CH₂—Ar), 4.96 (d, J=15.6 Hz, 1H, CH₂—Ar), 5.72 (s, 1H, H8′), 6.76 (s, 1H, H5′), 6.92 (dd, J=4.8, 3.1 Hz, 2H, H4, H7), 7.05 (ddd, J=8.0, 1.8, 0.9 Hz, 1H, H6), 7.16-7.18 (m, H3″, H5″), 7.42 (dd, J=8.6, 5.5 Hz, 2H, H2″, H6″). ¹³C NMR (DMSO-d₆, 150 MHz): δppm 21.0 (5-CH₃), 28.7 (C4′), 38.9 (C3′), 42.2 (CH₂—Ar), 55.8 (7′-OCH₃), 56.0 (6′-OCH₃), 63.6 (C3/C1′), 109.2 (C7), 109.4 (C8′), 113.0 (C5′), 115.8 (C3″, C5″), 125.5 (C4), 127.2 (C8′a), 129.3 (C6), 129.5 (C4′a), 129.9 (C2″, C6″), 132.2 (C3a), 133.5 (C1″), 135.5 (C5), 140.4 (C7a), 148.5 (C7′), 148.5 (C6′), 161.2 (C4″), 178.8 (C2). FTMS+cESI: m/z 433.191 [M+1]⁺.

### Example 41

6′,7′-Dimethoxy-5-methyl-1-(4-methylbenzyl)-3′,4′-dihydro-2′H-spiro[indoline-3,1′-isoquinolin]-2-one (5k).

Method G. Prepared from 5-methyl-1-(4-methylbenzyl)indoline-2,3-dione (1 g, 3.8 mmol), 3,4-dimethoxyphenethylamine (0.82 g, 4.5 mmol) and polyphosphoric acid (2 g). The crude product was purified by flash chromatography (hexane: ethyl acetate—60:40). Yield, 0.9 g, 56% (brown solid), M.p. 111-112° C.

¹H NMR (DMSO-d₆, 600 MHz): δppm 2.18 (s, 3H, 5-CH₃), 2.26 (s, 3H, 4″-CH₃), 2.70 (dt, J=15.9, 4.2 Hz, 1H, H4′a), 2.87 (ddd, J=15.2, 9.3, 5.4 Hz, 1H, H4′b), 3.04 (dt, J=12.5, 4.9 Hz, 1H, H3′a), 3.29 (s, 3H, 7′-OCH₃), 3.64 (m, 1H, H3′b), 3.73 (s, 3H, 6′-OCH₃), 4.69 (d, J=15.5 Hz, 1H, CH₂—Ar), 4.95 (d, J=15.5 Hz, 1H, CH₂—Ar), 5.73 (s, 1H, H8′), 6.75 (s, 1H, H5′), 6.86 (d, J=8.0 Hz, 1H, H7), 6.90 (d, J=1.8 Hz, 1H, H4), 7.03 (ddd, J=8.0, 1.8, 0.9 Hz, 1H, H6), 7.13 (d, J=7.7 Hz, 2H, H3″, H5″), 7.23-7.27 (m, 2H, H2″, H6″). ¹³C NMR (DMSO-d₆, 150 MHz): δppm 21.0 (5-CH₃), 21.1 (4″-CH₃), 28.7 (C4′), 38.9 (C3′), 42.8 (CH₂—Ar), 55.7 (7′-OCH₃), 55.9 (6′-OCH₃), 63.5 (C3/C1′), 109.3 (C7), 109.4 (C8′), 112.9 (C5′), 125.4 (C4), 127.3 (C8′a), 127.9 (C2″, C6″), 129.3 (C6), 129.5 (C4′a), 129.6 (C3″, C5″), 132.1 (C3a), 134.2 (C1″), 135.6 (C5), 137.1 (C4″), 140.6 (C7a), 147.5 (C7′), 148.4 (C6′), 178.7 (C2). FTMS+cESI: m/z, 429.21 [M+1]⁺.

### Example 42

6′,7′-Dimethoxy-5-nitro-3′,4′-dihydro-2′H-spiro[indoline-3,1′-isoquinolin]-2-one (5l).

Method G. Prepared from 5-nitroisatin (2.0 g, 10.4 mmol), 3,4-dimethoxyphenethylamine (2.3 g, 12.5 mmol) and polyphosphoric acid (5 g). The crude product was purified by flash chromatography (hexane:ethyl acetate—70:30). Yield, 2.9 g, 78% (brown solid), M.p. 174-175° C.

¹H NMR (DMSO-d₆, 600 MHz): δppm 2.69 (dt, J=15.9, 3.8 Hz, 1H, H4′a), 2.92 (ddd, J=15.5, 9.8, 5.5 Hz, 1H, H4′b), 2.97-3.04 (m, 1H, H3′a), 3.59.3.63 (m, 1H, H3′b), 3.34 (s, 3H, 7′-CH₃), 3.75 (s, 3H, 6′-CH₃), 5.88 (s, 1H, H8′), 6.79 (s, 1H, H5′), 7.11 (d, J=8.7 Hz, 1H, H7), 7.81 (d, J=2.4 Hz, 1H, H4), 8.22 (dd, J=8.7, 2.4 Hz, 1H, H6), 10.99 (s, 1H, H1), ¹³C NMR (DMSO-d₆, 150 MHz): δppm 28.5 (C4′), 38.5 (C3′), 55.9 (7′-OCH₃), 56.1 (6′-OCH₃), 63.6 (C3/C1′), 109.4 (C8′), 110.4 (C7), 113.1 (C5′), 120.5 (C4), 125.6 (C8′a), 126.7 (C6), 129.8 (C4′a), 137.0 (C3a), 142.8 (C7a), 147.6 (C7′), 148.8 (C6′), 149.4 (C5), 180.8 (C2). FTMS+cESI: m/z 356.12 [M+1]⁺.

### Example 43

2-(5,7-Dibromo-6′,7′-dimethoxy-2-oxo-3′,4′-dihydro-2′H-spiro[indoline-3,1′-isoquinolin]-1-yl)-N-phenylacetamide (5m).

Method G. Prepared from 2-(5,7-dibromo-2,3-dioxoindolin-1-yl)-N-phenylacetamide (2 g, 4.6 mmol), 3,4-dimethoxyphenethylamine (0.83 g, 4.6 mmol) and polyphosphoric acid (3 g). The crude product was purified by flash chromatography (hexane: ethyl acetate—60:40). Yield, 1.34 g, 49% (black solid); M.p. 202-203° C. (: MCl salt).

¹NMR (CD₃OD, 700 MHz): δppm 2.84 (dt, J=16.1, 4.6 Hz, 1H, H4′a), 3.02 (ddd, J=16.2, 8.9, 5.3 Hz, 1H, H4′b), 3.23 (dt, J=12.7, 4.8 Hz, 1H, H3′a), 3.67-3.74 (m, 4H, H3′b, 7′-OCH₃), 3.82 (s, 3H, 6′-OCH₃), 497 (m, 1H, CH₂—Ar), 5.02 (m, 1H, CH₂—Ar), 6.54 (s, 1H, H8′), 6.78 (s, 1H, H5′), 7.10 (m, 1H, H4″), 7.29-7.33 (m, 3H, H4, H3″, H5″), 7.54-7.56 (nn, 2H, H2″, H6″), 7.67(d, J=1.9 Hz, 1H, H6). ¹³C NMR (CD₃OD, 175 MHz): δppm 27.5 (C4′), 38.6 (C3′), 44.1 (CH₂—C═O), 55.0 (7′-OCH₃), 55.2 (6′-OCH₃), 63.1 (C3/C1′), 103.1 (C7), 109.7 (C8′), 111.9 (C5′), 115.7 (C5), 119.6 (C2″, C6″), 123.8 (C4″), 124.8 (C8′a), 127.0 (C4), 128.4 (C4′a), 128.5 (C3″, C5″), 136.1 (C6), 138.3 (C1″), 139.1 (C3a), 139.9 (C7a), 148.1 (C7′), 148.8 (C6′), 166.1 (CH₂—C═O), 179.3 (C2). FTMS+cESI: m/z 602.01 [M+1]⁺.

### Example 44

2′-(4-Fluorobenzyl)-6′,7′-dimethoxy-1-(4-methylbenzyl)-3′,4′-dihydro-2′H-spiro[indoline-3,1′-isoquinolin]-2-one (5n).

Method H. Prepared from previously synthesised 6′,7′-dimethoxy-1-(4-methylbenzyl)-3′,4′-dihydro-2′H-spiro[indoline-3,1′-isoquinolin]-2-one (5e) (1 g, 2.4 mmol) and 4-fluorobenzylchloride (0.7 g, 4.8 mmol). An acetonitrile (10 mL) solution of 5e and K₂CO₃ (0.7 g, 4.8 mmol) was stirred at room temperature for 1 hour. Thereafter, 4-fluorobenzylchloride (0.51 g, 3.6 mmol) and KI (0.08 g, 0.48 mmol) was added and reaction mixture heated at 80° C. for 2 hours. Upon completion of the reaction mixture, the acetonitrile was removed under reduced pressure; Viscous mass obtained was made basic to pH 10 by the addition of an aqueous solution of Na₂CO₃. Product was extracted into dichloromethane (30 mL×3), combined organic extracts dried over anhydrous sodium sulphate and concentrated under reduced pressure. The crude product was purified by flash chromatography (hexane: ethyl acetate—70:30). Yield, 1 g, 75% (yellow oil).

¹H NMR (DMSO-d₆, 700 MHz): δppm 2.26 (s, 3H, 4″-CH₃), 2.71 (dt, J=15.7, 3.3 Hz, 1H, H4′a), 2.75 (ddd, J=11.9, 5.4, 2.9 Hz, 1H, H3′a), 2.88 (ddd, J=16.0, 10.7 5.5, Hz, 1H, H4′b), 3.22 (m, 4H, N1′-CH₂, 7′-OCH₃), 3.29 (d, J=4.0 Hz, 1H, N1′-CH₂), 3.54 (td, J=11.1, 3.8 Hz, 1H, H3′b), 3.73 (s, 3H, 6′-OCH₃), 4.79 (d, J=15.2 Hz, 1H, N1-CH₂), 5.01 (d, J=15.2 Hz, 1H, N1-CH₂), 5.69 (s, 1H, H8′), 6.77 (s, 1H, H5′), 7.05 (td, J=7.52, 1.0 Hz, 1H, H5), 7.09-7.12 (m, 3H, H7, H3″′, H5′″), 7.19 (dd, J=7.4, 1.3 Hz, 1H, H3″, H5″), 7.29 (ddd, J=8.5, 3.9, 2.6 Hz, 3H, H6, H2″′, H6″′), 7.30-7.32 (m, 2H, H2″, H6″). ¹³C NMR (DMSO-d₆, 175 MHz): δppm 21.0 (4″-CH₃), 28.9 (C4′), 42.7 (C3′), 42.9 (N1-CH₂), 54.0 (N1′-CH₂), 55.6 (7′-OCH₃), 55.9 (6′-OCH₃), 68.9 (C3/C1′), 109.7 (C8′), 109.8 (C7), 112.4 (C5′), 115.4 (2C, C3″′, C5″′), 123.8 (C5), 124.7 (C4), 126.7 (C8′a), 128.2 (2C, C2″, C6″), 128.5 (C4′a), 129.6 (C6), 129.7 (2C, C3″, C5″), 130.2 (2C, C2″′, C6″′), 133.5 (C3a), 134.2 (C1″), 135.5 (C1″′), 137.4 (C4″), 143.7 (C7a), 147.5 (C7′), 148.5 (C6′), 161.1 (C4″′), 177.2 (C2). FTMS+cESI: m/z 523.24 [M+1]⁺.

### Example 45

6′,7′-Dimethoxy-1,2′-bis(4-methylbenzyl)-3′,4′-dihydro-2′H-spiro[indoline-3,1′-isoquinolin]-2-one (5o)

Method H. Prepared from 6′,7′-dimethoxy-1-(4-methylbenzyl)-3′,4′-dihydro-2′H-spiro[indoline-3,1′-isoquinolin]-2-one (5e) (1 g, 2.4 mmol) and 4-methylbenzylchloride (0.51 g, 3.6 mmol). The crude product was purified by flash chromatography (hexane: ethyl acetate—70:30). Yield, 0.8 g, 65% (yellow oil).

¹H NMR (DMSO-d₆, 700 MHz): δppm 2.25 (s, 6H, 4″-CH₃, 4′″-CH3), 2.69 (dt, J=15.8, 3.4 Hz, 1H, H4′a), 2.75 (dt, J=5.7, 3.0 Hz, 1H, H3′a), 2.86 (ddd, J=16.0, 10.7 5.6, Hz, 1H, H4′b), 3.21 (m, 5H, N2′-CH₂, 7′-OCH₃), 3.51-3.55 (m, 1H, H3′b), 3.72 (s, 3H, 6′-OCH₃), 4.79 (d, J=15.2 Hz, 1H, N1-CH₂), 5.01 (d, J=15.3 Hz, 1H, N1-CH₂), 5.70 (s, 1H, H8′), 6.76 (s, 1H, H5′), 7.05 (td, J=7.5, 1.0 Hz, 1H, H5), 7.09 (t, J=8.0 Hz, 3H, H7, H3′″, H5′″), 7.11-7.15 (m, 4H, H3″, H5″, H2″′, H6′″), 7.18-7.20 (m, 1H, H4), 7.29 (td, J=7.7, 1.3 Hz, 1H, H6), 7.30-7.33 (m, 2H, H2″′, H6′″). ¹³C NMR (DMSO-d₆, 175 MHz): δppm 21.0 (2C, 4″-CH₃, 4″′-CH₃), 28.9 (C4′), 42.5 (C3′), 42.9 (N1-CH₂), 54.5 (N2′-CH₂), 55.6 (7′-OCH₃), 55.9 (6′-OCH₃), 68.9 (C3/C1′), 109.7 (C8′), 109.8 (C7), 112.4 (C5′), 123.8 (C5), 124.7 (C4), 126.7 (C8′a), 127.0 (C4′a), 128.2 (2C, C2″, C6″), 128.4 (2C, C3″′, C5″′), 129.1 (C6), 129.3 (2C, C2′″, C6′″), 129.6 (C4″′), 129.7 (2C, C3″, C5″), 133.6 (C3a), 134.2 (C1″), 136.6 (C1′″), 137.4 (C4″), 143.7 (C7a), 147.5 (C7′), 148.5 (C6′), 177.2 (C2). FTMS+cESI: m/z 519.26 [M+1]⁺.

### Example 46

6′,7′-Dimethoxy-2′-methyl-1-(4-methylbenzyl)-3′,4′-dihydro-2′H-spiro[indoline-3,1′-isoquinolin]-2-one (5p). Method I

Prepared from previously synthesized 6′,7′-dimethoxy-1-(4-methylbenzyl)-3′,4′-dihydro-2′H-spiro[indoline-3,1′-isoquinolin]-2-one (5e) (1 g, 2.4 mmol) and formaldehyde (0.3 mL, of 37% formalin, 3.6 mmol, 1.5 eq). To a formic acid solution of 5e was added drop wise formaldehyde. reaction mixture was heated at 60° C. for 3 hours. Upon completion of the reaction mixture, reaction mixture was allowed to cool to room temperature, made basic by slow addition of 2 M aqueous sodium hydroxide to pH 10. Product was extracted into ethyl acetate (30 mL×3), combined organic extracts dried over anhydrous sodium sulphate and concentrated under reduced pressure. The crude product was purified by flash chromatography (hexane: ethyl acetate—50:50). Yield, 0.8 g, 78% (yellow oil).

¹H NMR (DMSO-d₆, 700 MHz): δppm 2.06 (s, 3H, N2′-CH₃), 2.26 (s, 3H, 4″-CH₃), 2.78 (dt, J=15.8, 3.6 Hz, 1H, H4′a), 2.88 (ddd, J=11.3, 5.7, 2.9 Hz, 1H, H3′a), 3.03 (ddd, J=16.0, 10.5, 5.6 Hz, 1H, 1H, H4′b), 3.22 (s, 3H, 7′-OCH₃), 3.63 (td, J=10.9, 4.1 Hz, 1H, H3′b), 3.73 (s, 3H, 6′-OCH₃), 4.77 (d, J=15.4 Hz, 1H, N1-CH₂), 4.97 (d, J=15.5 Hz, 1H, N1-CH₂), 5.65 (s, 1H, H8′), 6.77 (s, 1H, H5′), 6.99-7.01 (m, 2H, H4, H5), 7.06 (d, J=7.9 Hz, 1H, H7), 7.14 (d, J=7.8 Hz, 2H, H3″, H5″), 7.26-7.29 (in, 3H, H6, H2″, H6″). ¹³C NMR (DMSO-d₆, 175 MHz): δppm 21.0 (4″-CH₃), 28.7 (C4′), 39.6 (N2′-CH₃), 42.7 (N1-CH₂), 46.9 (C3′), 55.6 (7′-OCH₃), 55.9 (6′-OCH₃), 69.0 (C3/C1′), 109.6 (C8′), 109.6 (C7), 112.4 (C5′), 123.5 (C5), 124.8 (C4), 126.7 (C8′a), 128.0 (C4′a), 128.1 (2C, C2″, C6″), 129.4 (2C, C3″, C5″), 129.7 (C6), 133.2 (C3a), 134.2 (C1″), 137.3 (C4″), 143.5 (C7a), 147.4 (C7′), 148.5 (C6′), 177.3 (C2). FTMS+cESI: m/z 429.21 [M+1]⁺.

### Example 47

N-Ethyl-6′,7′-dimethoxy-1-(4-methylbenzyl)-2-oxo-3′,4′-dihydro-2′H-spiro[indoline-3,1′-isoquinoline]-2′-carboxamide (5q). (Method J)

New Method 3: Prepared from 6′,7′-dimethoxy-1-(4-methylbenzyl)-3′,4′-dihydro-2′H-spiro[indoline-3,1′-isoquinolin]-2-one (5e) (1 g, 2.4 mmol) and ethyl isocyanate (0.21 g, 0.23 mL, 2.9 mmol, 1.2 eq). An acetonitrile solution of 5e and ethylisocyanate was heated to 60° C. for 2 hours. Upon completion of the reaction mixture, reaction mixture was allowed to cool to room temperature, made basic by slow addition of aqueous sodium bicarbonate to pH 10. Product was extracted into ethyl acetate (30 mL×2), combined organic extracts dried over anhydrous sodium sulphate and concentrated under reduced pressure. The crude product was purified by flash chromatography (hexane: ethyl acetate—70:30). Yield, 0.6 g, 50% (white solid). M.p. 193-194° C.

¹H NMR (DMSO-d₆, 700 MHz): δppm 0.96 (t, J=7.17 Hz, 3H, N1′″-CH₂CH₃), 2.27 (s, 3H, 4″-CH₃), 2.90 (ddd, J=15.4, 4.8, 3.4 Hz, 1H, H4′a), 2.92-3.01 (m, 3H, 1H, H4′b), N1′″-CH₂CH₃), 3.11 (s, 3H, 7′-OCH₃), 3.55-3.60 (m, 1H, H3′a), 3.72 (s, 3H, 6′-OCH₃), 3.97 (td, J=12.2, 4.6 Hz, 1H, H3′b), 4.62 (d, J=15.5 Hz, 1H, N1-CH₂), 4.96 (d, J=15.5 Hz, 1H, N1-CH₂), 5.76 (s, 1H, H8′), 6.84 (s, 1H, H5′), 6.89 (td, J=7.5, 1.0 Hz, 1H, H5), 6.93 (dt, J=7.9, 0.7 Hz, 1H, H7), 7.03 (dd, J=7.3, 1.25 Hz, 1H, H4), 7.10-7.13 (d, J=7.8 Hz, 2H, H3″, H5″), 7.17 (td, J=7.7, 1.3 Hz, 1H, H6), 7.31-7.34 (m, 2H, H2″, H6″). ¹³C NMR (DMSO-d₆, 175 MHz): δppm 15.8 (N1′″-CH₂CH₃), 21.1 (4″-CH₃), 30.0 (C4′), 35.4 (N1′″-CH₃), 42.2 (C3′), 43.4 (N1-CH₂), 55.4 (7′-OCH₃), 55.9 (6′-OCH₃), 65.5 (C3/C1′), 108.9 (C7), 109.1 (C8′), 112.3 (C5′), 122.3 (C4), 122.7 (C5), 126.5 (C8′a), 128.2 (C4′a), 128.3 (2C, C2″, C6″), 128.9 (C6), 129.5 (2C, C3″, C5″), 134.5 (C1″), 135.7 (C3a), 137.0 (C4″), 143.5 (C7a), 147.7 (C7′), 148.3 (C6′), 156.7 (C2′″) 177.3 (C2). FTMS+cESI: m/z 486.24 [M+1]⁺.

### Example 48

6′,7′-Dimethoxy-2′-methyl-5-nitro-3′,4′-dihydro-2′H-spiro[indoline-3,1′isoquinolin]-2-one (5r).

Method I: Prepared from 6′,7′-dimethoxy-5-nitro-3′,4′-dihydro-2′H-spiro[indoline-3,1′-isoquinolin]-2-one (5l). (1 g, 2.8 mmol) and formaldehyde (0.5 mL of 37% formalin, 4.2 mmol, 1.5 eq). The crude product was purified by flash chromatography (hexane: ethyl acetate—50:50). Yield, 0.6 g, 58% . (brown solid). M.p. 137-138° C.

¹H NMR (DMSO-d₆, 700 MHz): δppm 2.10 (s, 3H, N2′-CH₃), 2.80 (dt, J=16.7, 3.7 Hz, 1H, H4′a), 2.91 (m, 1H, H3′a), 3.02-3.10 (m, 1H, H4′b), 3.39 (s, 3H, 7′-OCH₃), 3.49-3.543.63 (m, 1H, H3′b), 3.74 (s, 3H, 6′-OCH₃), 5.84 (s, 1H, H8′), 6.81 (s, 1H, H5′), 7.14 (d, J=8.7 Hz, 1H, H7), 7.70 (d, J=2.4 Hz, 1H, H4), 8.25 (dd, J=8.8, 2.4 Hz, 1H, H6), 11.12 (s, 1H, H1). ¹³C NMR (DMSO-d₆, 175 MHz): δppm 28.6 (C4′), 39.6 (N2′-CH₃), 46.94 (C3′), 55.9 (7′-OCH₃), 56.1 (6′-OCH₃), 69.2 (C3/C1′), 109.6 (C7), 110.6 (C8′), 112.5 (C5′), 120.3 (C4), 125.3 (C8′a), 126.6 (C6), 128.5 (C4′a), 135.0 (C3a), 143.2 (C7a), 147.7 (C7′), 148.8 (C6′), 149.6 (C5), 177.3 (C2). FTMS+cESI: m/z 370.14 [M+1]⁺.

### Example 49

5-Amino-6′,7′-dimethoxy-3′,4′-dihydro-2′H-spiro[indoline-3,1′-isoquinolin]-2-one (5s).

Method K. Compound 5s was obtained from the reduction of the 5-nitro group of previously synthesized 51 (1.0 g, 2.2.8 mmol) and zinc dust (0.64 g, 9.8 mmol, 3.5 eq). To a warm ethanolic solution of 5l were added portions of zinc dust and concentrated HCl over 2-minute intervals. Upon complete addition of the reagents, the reaction mixture was refluxed for 1 hour. During this time there was complete consumption of 5l as observed on TLC. Reaction mixture was concentrated under reduced pressure, made basic to pH 9 by the addition of saturated aqueous sodium bicarbonate. Within the process, insoluble zinc carbonate was precipitated out and filtered off by suction filtration. Product was extracted into ethyl acetate (30 mL×2), combined organic extracts dried over anhydrous sodium sulphate and concentrated under reduced pressure. The crude product was purified by flash chromatography (hexane: ethyl acetate—20:80). Yield, 0.8 g, 89% (brown solid). M.p. 186-187° C.

¹H NMR (DMSO-d₆, 700 MHz): δppm 2.63 2.70 (m, 1H, H4′a), 2.80 (ddd, J=15.9. 8.6, 5.3 Hz, 1H, H4′b), 2.98 (dt, J=12.4, 5.1 Hz, 1H, H3′a), 3.57 (ddd, J=12.7, 8.7, 4.4 Hz, 1H, H3′b), 3.42 (s, 3H, 7′-OCH₃), 3.73 (s, 3H, 6′-OCH₃), 5.92 (s, 1H, H8′), 6.34 (d, J=2.3 Hz, 1H, H4), 6.42 (dd, J=8.2, 2.3 Hz, 1H, H6), 6.59 (d, J=8.2 Hz, 1H, H7), 6.71 (s, 1H, H5′), 9.87 (s, 1H, H1). ¹³C NMR (DMSO-d₆, 175 MHz): δppm 28.8 (C4′), 38.8 (C3′), 55.9 (7′-OCH₃), 56.1 (6′-OCH₃), 64.1 (C3/C1′), 110.0 (C8′), 110.41 (C7), 112.0 (C4), 112.8 (C5′), 113.8 (C6), 128.0 (C8′a), 129.3 (C4′a), 132.0 (C7a), 137.2 (C3a), 144.4 (C5), 147.4 (C7′), 148.4 (C6′), 180.2 (C2). FTMS+cESI: m/z 324.13 [M−1]⁺.

### Example 50

5,6-Difluoro-6′,7′-dimethoxy-3′,4′-dihydro-2′H-spiro[indoline-3,1′-isoquinolin]-2-one(5t).

Method G. Prepared from 5,6-difluoroisatin (1.0 g, 5.5 mmol, 1 eq), 3,4-dimethoxyphenethylamine (1.2 g, 6.6 mmol) and polyphosphoric acid (5 g). The crude product was purified by flash chromatography (hexane: ethyl acetate—70:30). Yield, 1.3 g, 68% (white solid), M.p. 220-221° C.

¹H NMR (DMSO-d₆, 700 MHz): δppm 2.69 (dt, J=15.9, 4.1 Hz, 1H, H4′a), 2.84 (ddd, J=15.2, 9.3, 5.4 Hz, 1H, H4′b), 2.98 (dt, J=11.4, 4.7 Hz, 1H, H3′a), 3.45 (s, 3H, 7′-OCH₃), 3.57-3.64 (m, 1H, H3′b), 3.74 (s, 3H, 6′-OCH₃), 5.88 (s, 1H, H5′), 6.75 (s, 1H, H8′), 6.93 (dd, J=10.5, 6.6 Hz, 1H, H7), 7.09 (dd, J=9.8, 7.9 Hz, 1H, H4), 10.40 (s, 1H, H1). ¹³C NMR (DMSO-d₆, 175 MHz): δppm 28.5 (C4′), 38.5 (C3′), 55.9 (7′-OCH₃), 56.1 (6′-OCH₃), 63.8 (C3/C1′), 99.8 (C7), 109.5 (C5′), 113.0 (C8′), 114.6 (C4), 126.3 (C8′a), 129.6 (C4′a), 132.1 (C3a), 139.3 (C7a), 147.6 (C7′), 148.8 (C6′), 145.2 (C5), 149.4 (C6), 180.5 (C2). FTMS+cESI: m/z 347.12 [M+1]⁺.

### Example 51

5-Fluoro-6′,7′-dimethoxy-3′,4′-dihydro-2″H-spiro[indoline-3,1′-isoquinolin]-2-one (5u)

Method G. Prepared from 5-fluoroisatin (1.0 g, 6.1 mmol, 1 eq), 3,4-dimethoxyphenethylamine (1.3 g, 7.3 mmol, 1.2 eq) and polyphosphoric acid (2 g). The crude product was purified by flash chromatography (hexane: ethyl acetate—70:30). Yield, 1.1 g, 55% (brown solid), M.p. 98-99° C.

¹H NMR (DMSO-d₆, 600 MHz): δppm 2.67 (dt, J=15.9, 4.2 Hz, 1H, H4′a), 2.84 (ddd, J=15.2, 9.1, 5.3 Hz, 1H, H4′b), 2.99 (ddd, J=12.5, 5.4, 4.1 Hz, 1H, H3′a), 3.57-3.63 (m, 1H, H3′b), 3.43 (s, 3H, 7′-OCH₃), 3.74 (s, 3H, 6′-OCH₃), 5.887 (s, 1H, H8′), 6.75 (s, 1H, H5′), 6.85-6.91 (m, 2H, H4, H7), 7.06 (dd, J=9.6, 8.5, 2.7 Hz, 1H, H6), 10.31 (s, 1H, H1). ¹³C NMR (DMSO-d₆, 150 MHz): δppm 28.6 (C4′), 38.6 (C3′), 55.9 (7′-OCH₃), 56.1 (6′-OCH₃), 64.2 (C3/C1′), 109.5 (C8′), 110.8 (C7), 112.6 (C5′), 113.0 (C4), 115.5 (C6), 126.6 (C8′a), 129.6 (C4′a), 138.8 (C7a), 147.5 (C7′), 148.6 (C6′), 157.8 (C3a), 159.3 (C5), 180.5 (C2). FTMS+cESI: m/z 329.13 [M+1]⁺.

### Example 52

6′,7′,8′-Trimethoxy-3′,4′-dihydro-2′H-spiro[indoline-3,1′-isoquinolin]-2-one (6a).

Method G. Prepared from isatin (1.6 g, 10.9 mmol) and 3,4,5-trimethoxyphenethylamine, 11b, (2.8 g, 13 mmol) in polyphosphoric acid (3 g). The crude product was purified by flash chromatography (hexane: ethyl acetate—80:20). Yield, 1.24 g, 34% (white solid). M.p. 238-240° C.

¹H NMR (CD₃OD, 600 MHz): δppm 2.85-2.97 (m, 2H, H4′a, H4′b), 3.21 (s, 3H, 8′-OCH₃), 3.26-3.30 (m, 2H, H3′a, H3′b), 3.69 (s, 3H, 7′-OCH₃), 3.86 (s, 3H, 6′-OCH₃), 6.65 (s, 1H, H5′), 6.96 (dt, J=7.8, 0.8 Hz, H7), 6.99 (dt, td, J=7.8, 0.8 Hz, 1H, H4), 7.06 (ddd, J=7.4, 1.2, 0.6 Hz, 1H, H5), 7.25 (td, J=7.7, 1.3 Hz, 1H, H6). ¹³C NMR (CD₃OD, 150 MHz): δppm 28.0 (C4′), 38.6 (C3′), 55.0 (6′-OCH₃), 58.5 (8′-OCH₃), 59.5 (7′-OCH₃), 62.4 (C3/C1′), 107.5 (C5′), 109.7 (C7), 120.4 (C8′a), 121.7 (C4), 121.7 (C5), 128.4 (C6), 132.5 (C4′a), 135.1 (C3a), 140.0 (C7a), 142.2 (C6′), 150.1 (C8′), 153.2 (C7′), 181.0 (C2). FTMS+cESI: m/z 341.15 [M+1]⁺

### Example 53

5-Chloro-6′,7′,8′-trimethoxy-3′,4′-dihydro-2′H-spiro[indoline-3,1′-isoquinolin]-2-one (6b).

Method G. From 5-chloroisatin (1.5 g, 8.3 mmol) and 3,4,5-trimethoxyphenethylamine, 11b, (2.1 g, 9.9 mmol)] in polyphosphoric acid (3 g). Purified by flash chromatography (hexane: ethyl acetate—80:20). Yield, 1.8 g, 58% (brown solid). M.p. 102-105° C.

¹H NMR (CD₃OD, 600 MHz): δppm 2.86-2.98 (m, 2H, H4′a, H4′b), 3.18-3.24 (m, 1H, H3′a,), 3.28-3.32 (m, 4H, H3′b , 8′-OCH₃), 3.70 (s, 3H, 7′-OCH₃), 3.87 (s, 3H, 6′-OCH₃), 6.65 (s, 1H, H5′), 6.96 (dt, J=8.3, 1H, H7), 7.05 (d, td, J=2.1 Hz, 1H, H4), 7.26 (dd J=8.3, 2.1 Hz, 1H, H6).

¹³C NMR (CD₃OD, 150 MHz): δppm 27.9 (C4′), 38.5 (C3′), 55.1 (6′-OCH₃), 58.6 (8′-OCH₃), 59.5 (7′-OCH₃), 62.4 (C3/C1′), 107.5 (C5′), 110.7 (C7), 119.6 (C8′a), 123.9 (C4), 126.8 (C5), 128.2 (C6), 132.6 (C4′a), 137.0 (C3a), 140.0 (C8′), 140.9 (C7a), 150.0 (C6′), 153.4 (C7′), 181.0 (C2). FTMS+cESI: m/z 375.11 [M+1]⁺

### Example 54

5,7-Dibromo-6′,7′,8′-trimethoxy-3′,4′-dihydro-2′H-spiro[indoline-3,1′-isoquinolin]-2-one (6c).

Method G. From 5,7-dibromoisatin, 7c, (1.5 g, 5.0 mmol) and 3,4,5-trimethoxyphenethylamine, 11b, (1.3 g, 6 mmol) in polyphosphoric acid (2 g). The crude product was purified by flash chromatography (hexane: ethyl acetate—80:20). Yield, 2.1 g, 84% (brown solid). M.p. 200-203° C.

¹H NMR (CD₃OD, 600 MHz): δppm 2.89 (td, J=6.5, 5.5, 2H, H4′a, H4′b), 3.18 (ddd, J=13.1, 7.4, 5.6, 1H, H3′a,), 3.27-3.31 (m, 1H, H3′b), 3.37 (s, 8′-OCH₃), 3.70 (s, 3H, 7′-OCH₃), 3.87 (s, 3H, 6′-OCH₃), 6.65 (s, 1H, H5′), 7.15 (d, J=1.8 Hz, 1H, H4), 7.60 (d, J=1.8, 1H, H6). ¹³C NMR (CD₃OD, 150 MHz): δppm 27.8 (C4′), 38.4 (C3′), 55.1 (6′-OCH₃), 58.6 (8′-OCH₃), 59.5 (7′-OCH₃), 61.3 (C3/C1′), 102.8 (C7), 107.5 (C5′), 114.0 (C5), 119.2 (C8a), 125.6 (C4), 132.7 (C4′a), 133.1 (C6), 138.4 (C3a), 139.9 (C8′), 141.2 (C7a), 150.0 (C6′), 153.6 (C7′), 179.8 (C2). FTMS+cESI: m/z 498.97 [M+1]⁺

### Example 55

1-(4-Fluorobenzyl)-6′,7′,8′-trimethoxy-3′,4′-dihydro-2′H-spiro[indoline-3,1′-isoquinolin]-2-one (6d).

Method G. Prepared from 1-(4-fluorobenzyl)indoline-2,3-dione, 8a,(2.0 g, 7.8 mmol), 3,4,5-trimethoxyphenethylamine, 11b, (1.7 g, 7.8 mmol] and polyphosphoric acid (2.5 g). The crude product was purified by flash chromatography (hexane: ethyl acetate—80:20). Yield, 3.0 g, 62% (white solid). M.p, 173-175° C. ¹H NMR (CD₃OD, 600 MHz): δppm 2.87 (s, 3H, 8′-OCH₃), 2.89-3.0 (m, 2H, H4′a, H4′b), 3.27 (ddd, J=13.2, 7.7, 5.1 Hz, 1H, H3′a), 3.36 (m. 1H, H3′b), 3.70 (s, 3H, 7′-OCH₃), 3.87 (s, 6′-OCH₃), 4.91 (d, J=15.6 Hz, 1H, —CH₂—Ar), 5.08 (d, J=15.6 Hz, 1H, CH₂—Ar), 6.67 (s, 1H, H5′), 6.95 (dt, J=7.8, 0.7 Hz, 1H, H7), 7.01 (td, J=7.5, 1.0 Hz, 1H, H5), 7.08-7.13 (m, 3H, H4, H3″, H5″), 7.25 (td, J=7.8, 1.3 Hz, 1H, H6), 7.54-7.58 (m, 2H, H2″, H6″). ¹³C NMR (CD₃OD, 150 MHz): δppm 28.1 (C4′), 38.7 (C3′), 42.7 (—CH₂—Ar), 55.1 (6′-OCH₃), 58.3 (8′-OC11.3),59.5 (7′-OCH₃) 62.0 (C3/C1′), 107.6 (C5′), 109.2 (C7), 114.9 (2C, C3″, C5″), 120.0 (C8′a), 122.4 (C4), 123.4 (C5), 128.4 (C6), 127.6 (2C, C2″, C6″), 132.2 (C3a), 132.8 (C4′a), 134.7 (C1″), 140.1 (C8′), 142.8 (C7a), 150.0 (C7′), 153.3 (C6′), 163.2 (C4″), 178.9 (C2). FTMS+cESI: m/z 430.19 [M+1]⁺.

### Example 56

6′,7′,8′-Trimethoxy-1-(4-methylbenzyl)-3′,4′-dihydro-2′H-spiro[indoline-3,1′-isoquinolin]-2-one (6e).

Method G. Prepared from 1-(4-methylbenzyl)indoline-2,3-dione, 8e, (1.6 g, 6.4 mmol), 3,4,5-trimethoxyphenethylamine, 11b, (1.6 g, δmmol)] and polyphosphoric acid (2 g). The crude product was purified by flash chromatography (hexane: ethyl acetate—80:20). Yield, 2.3 g, 80% (brown solid). M.p. 135-138° C. ¹H NMR (CD₃OD, 600 MHz): δppm 2.34 (s, 3H, 4″-CH₃-Bz), 2.89 (s, 3H, 8′-OCH₃), 2.90-2.96 (m, 2H, H4′a, H4′b), 3.24-3.30 (m, 1H, H3′a), 3.34-3.37 (m. 1H, H3′b), 3.71 (s, 3H, 7′-OCH₃), 3.87 (s, 4H, H3′b, 6′-OCH₃), 4.87 (d, J=15.5 Hz, 1H, —CH₂—Ar), 5.06 (d, J=15.5 Hz, 1H, CH₂—Ar), 6.67 (s, 1H, H5′), 6.92 (d, J=7.9 Hz, 1H, H7), 6.99 (td, J=7.6, 1.0 Hz, 1H, H5), 7.11 (dd, J=7.4, 1.2 Hz, 1H, H4), 7.17-7.20 (m, 2H, H3″H5″), 7.22 (td, J=7.8, 1.3 Hz, 1H, H6), 7.40-7.42 (m, 2H, H2″, H6″). ¹³C NMR (CD₃OD, 150 MHz): δppm 19.8 (4″-CH₃), 28.1 (C4′), 38.7 (C3′), 43.3 (—CH₂—Ar), 55.1 (6′-OCH₃), 58.3 (8′-OCH₃), 59.5 (7′-OCH₃) 62.0 (C3/C1′), 107.5 (C5′), 109.4 (C7), 120.1 (C8′a), 122.3 (C5), 123.3 (C4), 127.6 (2C, C2″, C6″), 128.8 (2C, C3″, C5″), 132.7 (C4′a), 133.1 (C3a), 134.1 (C1″), 137.1 (C4″), 140.1 (C8′), 143.0 (C7a), 150.1 (C7′), 153.2 (C6′), 178.9 (C2). FTMS+cESI: m/z 445.21 [M+1]⁺

### REFERENCES

Acikgoz E, Guven U, Duzagac F, Uslu R, Kara M, Soner B C, Oktem G (2015) Enhanced G2/M Arrest, Caspase Related Apoptosis and Reduced E-Cadherin Dependent Intercellular Adhesion by Trabectedin in Prostate Cancer Stem Cells. PLoS One. 10(10):e0141090. doi: 10.1371/journal.pone.0141090. eCollection 2015.

Brouwer W G, Craig W A, Jeffreys J A D & Munro A. (1972) The reaction between isatin and some amines. J. Chem, Soc., Perkin Trans. 1, 0, 124-129.

Chen D, Shen A, Fang G, Liu H, Zhang M, Tang S, Xiong B, Ma L, Geng M, Shen J. (2016) Tetrahydroisoquinolines as novel histone deacetylase inhibitors for treatment of cancer. Acta PharmaceuticaSinicaB. 6(1):93-99.

Chiba K, Adachi K. (2012) Sphingosine 1-phosphate receptor 1 as a useful target for treatment of multiple sclerosis. Pharmaceuticals 5: 514-528.

Chu W, Rothfuss J, d'Avignon A, Zeng C, Zhou D, Hotchkiss R S, Mach R H. (2007) Isatinsulfonamide analogs containing a Michael addition acceptor: A new class of caspase 3/7 inhibitors. J. Med. Chem. 2007, 50, 3751-3755.

Chu W, Zhang J, Zeng C, Rothfuss J, Tu Z, Chu Y, Reichert DE, Welch M J, Mach R H. (2005) N-Benzylisatinsulfonamide analogues as potent caspase-3 inhibitors: Synthesis, in vitro activity, and molecular modeling studies. J. Med. Chem. 48, 7637-7647.

Dandia A, Saini D, Saini D K, Mathur M (2014) A facile green synthesis process of 3-substituted-hydroxyindolin-2-ones and their in vitro antioxidant and antimicrobial potential. Eur. Chem. Bull. 3(1), 85-92.

Davidovich P, Aksenova V, Petrova V, Tentler D, Orlova D, Smirnov S, Gurzhiy V, Okorokov A L, Garabadzhiu A, Melino G, Barlev N, Tribulovich V (2015) Discovery of novel isatin-based p53 inducers ACS Med. Chem. Lett., 6, 856-860.

Davis H J, Kavanagh M E, Balan T, Abell C, Coyne AG (2016) Spirooxindoles as novel 3D-fragment scaffolds: Synthesis and screening against CYP121 from M. tuberculosis. Bioorg. Med. Chem. Lett. 26: 3735-3740.

DeBono A, Capuano B and Scammells P J (2015) Progress toward the development of noscapine and derivatives as anticancer agents. J. Med. Chem. 58, 5699-5727.

Evans B E, Riffle K E, Bock M G, DiPardo R M, Freidinger R M, Whitter W L, Lundell G F, Veber D F, Anderson P S, Chang R S (1988) Methods for drug discovery: development of potent, selective, orally effective cholecystokinin antagonists. J. Med. Chem. 31:2235-2246.

Gangapuram M, Eyunni S, Redda K K (2014) Synthesis and pharmacological evolution of tetrahydroisoquinolines as anti-breast cancer agents. J. Cancer Sci Ther. 6: 161-169.

Gao Y, Gao F, Chen K, Tian M-L, Zhao D-L. (2015) Sphingosine kinase 1 as an anticancer therapeutic target. Drug Des. Dev. Ther. 9:3239-3245.

Ghaly P E, Abou El-Magd R M, Churchill CDM, Tuszynski J A, West F G (2016) A new antiproliferative noscapine analogue: chemical synthesis and biological evaluation. Oncotarget 7:40518-40530.

GLOBOCAN 2012 (IARC) Section of Cancer Surveillance (Nov. 6, 2018.

Hientz K, Mohr A, Bhakta-Guha D, Efferth T (2017) The role of p53 in cancer drug resistance and targeted chemotherapy. Oncotarget 8 (5): 8921-8946.

Holzer P, Masuya M, Furet P, Kallen J, Valat-Stachyra T, Ferretti S, Berghausen J, Bouisset-Leonard M, Buschmann N, Pissot-Soldermann C, Rynn C, Ruetz S, Stutz S, Chène P, Jeay S, Gessier F. Discovery of a dihydroisoquinolinone derivative (NVP-CGM097): A highly potent and selective MDM2 inhibitor undergoing phase 1 clinical trials in p53wt tumors. J. Med. Chem. 2015, 58, 6348-6358.

Ishiba H, Noguchi T, Shu K, Ohno H, Honda K, Kondoh Y, Osada H, Fujii N, Oishi S. (2017) Investigation of the inhibitory mechanism of apomorphine against MDM2-p53 interaction. Bioorg Med. Chem. Lett. 27(11): 2571-2574.

Kametani T, Fukumoto K, Agui H, Yagi H, Kigasawa T K, Sugahara Hiiragi M, Hayasaka T, Ishimaru H. (1968) Phenolic Cyclisation. Part 1. Novel synthesis of 1-substituted isoquinoline and spiro[cycloalkane-1,1′-isoquinoline] derivatives and its application to the total synthesis of isoquinolinealkaloids. J. Chem. Soc.(C) 112-118.

Khan F A, Maalik A. (2015) Advances in Pharmacology of Isatin and its Derivatives: A Review. Trop. J. Pharm. Res. 14(10): 1937-1942.

Kumar D, Sharma V S, Kumar R, Singh T, Singh H, Singh A D, Roy R K (2013) Design, synthesis and anticonvulsant activity of some new 5,7-dibromoisatin semicarbazone derivatives. EXCLI J. 12: 628-640-ISSN 1611-2156.

Kumar R R, Perumal S, Senthilkumar P, Yogeeswari P, Sriram D. (2008) Discovery of antimycobacterial spiro-piperidin-4-ones: an atom economic, stereoselective synthesis, and biological intervention. J. Med. Chem: 51, 5731-5735.

Lazar C, Kluczyk A, Kiyota T, Konishi Y. (2004) Drug Evolution Concept in Drug Design: 1. Hybridization Method. J. Med. Chem. 47: 6973-6982.

Maiti A, Takabe K, Hait N C. (2017) Metastatic triple-negative breast cancer is dependent on SphKs/S1P signaling for growth and survival. Cell Signal. 32: 85-92.

Maresh JJ , Ralko A A, Speltz T E, Burke J L, Murphy C M, Gaskell Z, Girel J K, Terranova E, Richtscheidt C, Krzeszowiec M. (2014) Chemoselective Zinc/HCl Reduction of Halogenated β-Nitrostyrenes: Synthesis of Halogenated Dopamine Analogues. SYNLETT 25: 2891-2894.

Medvedev A, Buneeva O, Glover V. (2007) Biological targets for isatin and its analogues: Implications for therapy. Bioiogics: Targets & Therapy 2007:1(2) 151-162.

Misztal S, Paluchowska M H, Mokrosz M J, Bartyzel P, Mokrosz J L. (1993) Synthesis and stereochemistry of new trihydrodiazabicyclo[3.m.n.]alkano[4′,5′:1,2]pyrido[3,4-b]indole ring system [1]. J. Het. Chem. 30:1543-1548.

Modi N R, Shah R J, Patel M J, Suthar M, Cahauhan B F, Patel L J. (2011) Design, synthesis, and QSAR study of novel 2-(2,3-dioxo-2,3dihydro-1H-indol-1-yl)-N-phenylacetamide derivatives as cytotoxic agents. Med. Chem. Res. 20: 615-625.

Mohammadi S, Seyedhoseini F S, Asadi J, Yazdani Y. (2017) Effects of berberine on the secretion of cytokines and expression of genes involved in cell cycle regulation in THP-1 monocytic cell line. Iran J Basic Med Sci. 20(5):530-537.

Mohammed M M D, Ibrahim N A, Awad N E, Matloub A A, Mohamed-Ali A G, Barakat E E, Mohamed A E, Colla P L (2011) Anti-HIV-1 and cytotoxicity of the alkaloids of Erythrina abyssinica Lam. growing in Sudan. Nat. Prod. Res. 2011, 1-11.

Nesi G, Sestito S, Mey V, Ricciardi S, Falasca M, Danesi R, Lapucci A, Breschi M C, Fogli S, Simona Rapposelli S. (2013) Synthesis of novel 3,5-disubstituted-2-oxindole derivatives as antitumor agents against human non-small cell lung cancer. ACS Med. Chem. Lett. 4: 1137-1141.

Ngo Hanna J, Ntie-Kang F, Kaiser M, Brun R, Efange S M N. (2014) 1-Aryl-1,2,3,4-tetrahydroisoquinolines as potential antimalarials: synthesis, in vitro antiplasmodial activity and in silico pharmacokinetics evaluation. RSC Adv. 4, 22856. DOI: 10.1039/c3ra46791k.

Park S-J, Im D-S. (2017) Sphingosine 1-phosphate receptor modulators and drug discovery. BiomolTher 25(1), 80-90.

Patmanathan S N, Yap L F, Murray P G, Paterson I C (2015) The antineoplastic properties of FTY720: evidence for the repurposing of fingolimod, J. Cell. Mol. Med. 19(10):2329-2340.

Pawelczyk A, Sowa-Kasprzak K, Olender D, Zaprutko L. (2018) Molecular consortia—various structural and synthetic concepts for more effective therapeutics synthesis. Intl. J. Mol. Sci. 2018, 19, 1104; doi: 10.3390/ijms19041104.

Qing Z X, Huang J L, Yang X Y, Liu J H, Cao H L, Xiang F, Cheng P, Zeng J G (2017) Anticancer and reversing multidrug resistance activities of natural isoquinoline alkaloids and their structure-activity relationship. Curr Med Chem. 2017 Sep 20. doi: 10.2174/0929867324666170920125135.

Rosenberg A J, Liu H, Jin H, Yue X, Riley S, Brown S J, Tu Z. (2016) Design, synthesis, and in vitro and in vivo evaluation of an ¹⁸F-Labeled sphingosine 1-phosphate receptor 1 (S1P1) PET tracer. J. Med Chem. 59(13): 6201-6220.

Vermersch P. (2018) Sphingosine-1-phosphate receptor modulators in multiple sclerosis. Eur. Neurol. Rev. 13(1):25-30.

Viegas-Junior C, Danuello A, da Silva Bolzani V, Barreiro E J, Fraga CAM. (2007) Molecular Hybridization: A Useful Tool in the Design of New Drug Prototypes. Curr. Med. Chem. 14: 1829-1852.

Vine K L, Locke J M, Ranson M, Pyne S G, Bremner J B. (2007) An Investigation into the cytotoxicity and mode of action of some novel N-alkyl-substituted isatins. J. Med. Chem. 2007, 50 (21):5109-5117.

Vine, K. L., Matesic, L., Locke, J. M. , Skropeta, D. (2013). Recent highlights in the development of isatin-based anticancer agents. In M. Prudhomme (Eds.), Advances in Anticancer Agents in Medicinal Chemistry pp. 254-312.

Wang S, Liang Y, Chang W, Hu B, Zhang Y. (2018) Triple Negative Breast Cancer Depends on Sphingosine Kinase 1 (SphK1)/Sphingosine1-Phosphate (S1P)/Sphingosine 1-Phosphate Receptor 3 (S1PR3)/Notch Signaling for Metastasis. Med Sci Monit. 24: 1912-1923.

Watters R J, Wang H-G, Sung S-S, Loughran T P, Liu X (2011) Targeting Sphingosine-1-Phosphate Receptors in Cancer. Anticancer Agents Med Chem. 11(9): 810-817.

Welsch M E, Snyder S A, Stockwell B R (2010) Privileged scaffolds for library design and drug discovery. Curr. Opin. Chem. Biol. 14:1-15.

World Health Organization. Cancer Fact Sheet, February 2017.

Yu B, Yu D-Q, Liu H-M (2015) Spirooxindoles: Promising scaffolds for anticancer agents. Eur. J. Med. Chem. 97: 673-698.

Yu B, Zheng Y-Z, XShi X-J, Qi P-P, Liu H-M. (2016) Natural product-derived spirooxindolefragments serve as privileged substructures for discovery of new anticancer agents. Anti-Cancer AgentsMed. Chem. 16: 1315-1324.

Ze-hong MIAO, Jian-ming FENG, Jian DING (2009) Newly discovered angiogenesis inhibitors and their mechanisms of action. Acta PharmacologicaSinica (2012) 33: 1103-1111.

Zhao H & Dietrich J. (2015) Privileged scaffolds in lead generation. Expert Opin. Drug Discov. 10(7):781-790.

Zeng C, Rothfuss J M, Zhang J, Vangveravong S, Chu W, Li S, Tu Z, Xu J, Mach R H. (2014) Functional assays to define agonists and antagonists of the sigma-2 receptor. Anal. Biochem. 448: 68-74.

Zheng G-H, Shen J-J, Zhan Y-C, Yi H, Xue S-T, Wang Z, Ji X-Y, Li Z-R. (2014) Design, synthesis and in vitro and in vivo antitumour activity of 3-benzylideneindolin-2-one derivatives, a novel class of small-molecule inhibitors of the MDM2-53 interaction. European J. Med. Chem. 81: 277-288.

Zheng Y-J & Tice C M (2016) The utilization of spirocyclic scaffolds in novel drug discovery. Expert Opin. Drug Discov. 11(9): 831-834.

Zhou J, Gupta K, Aggarwal S, Aneja R, Chandra R, Panda D, And Joshi H C (2003) Brominated derivatives of noscapine are potent microtubule-interfering agents that perturb mitosis and inhibit cell proliferation. Mol. Pharmacol. 63:799-807.

Ziarani G M, Moradi R, Lashgari N (2016) Synthesis of spiro-fused heterocyclic scaffolds through multicomponent reactions involving isatin. ARKIVOC (i) 1-81.

Zinzi L, Capparelli E, Cantore M, Conlino M, Leopoldo M, Colabufo N A. (2014) Small and innovative molecules as new strategy to revert MDR. Front. Oncol.doi: 10.3389/fonc.2014.00002.

1. A compound according to the chemical structure I: 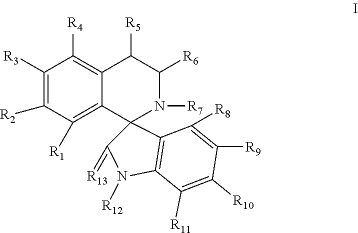
wherein
R₁ is H, OH, C₁-C₆ hydroxyalkyl, halo (F, Cl, Br, I), C₁-C₆ alkoxy (often C₁-C₃ alkoxy, more often OMe), (CH₂)ₙCOOH, (CH₂)ₙC(O)C₀-C₆ alkyl, (CH₂)ₙC(O)OC₁-C₆ alkyl, (CH₂)ₙOC(O)C₀-C₆ alkyl or O(CH₂)ₙaryl;
R₂ and R₃ are each independently H, OH, C₁-C₆ hydroxyalkyl, halo (F, Cl, Br, I), C₁-C₆ alkoxy (often C₁-C₃ alkoxy, more often OMe), (CH₂)ₙCOOH, (CH₂)ₙC(O)C₀-C₆ alkyl, (CH₂)ₙC(O)OC₁-C₆ alkyl, (CH₂)ₙOC(O)C₀-C₆ alkyl, O—(CH₂)ₙaryl, or R₂ and R₃ together form a 5- or 6-membered cycloalkyl or heterocyclic group containing 1, 2 or 3 heteroatoms (O, S, or N), preferably, the heterocyclic group formed is a dioxolanyl (3,4-methylenedioxy), dioxanyl (3,4-ethylenedioxy), dithiolanyl, tetrahydrofuranyl, tetrahydrothiophenyl, pyrrolidinyl, imidazolyl, imidazolinyl, imidazolidinyl, tetrahydropyranyl, thianyl, piperidinyl or piperazinyl;
R₄ is H, OH, C₁-C₆ hydroxyalkyl, halo (F, Cl, Br, I), C₁-C₆ alkoxy (often C₁-C₃ alkoxy, more often OMe), (CH₂)ₙCOOH, (CH₂)ₙC(O)C₀-C₆ alkyl, (CH₂)ₙC(O)OC₁-C₆ alkyl, (CH₂)ₙOC(O)C₀-C₆ alkyl, O(CH₂)ₙaryl, (CH₂)ₙAryl (often, phenyl or naphthyl, more often phenyl), (CH₂)ₙHeteroaryl;
R₅ is H, alkyl (preferably C₁-C₆ alkyl), C₁-C₆ alkoxy, (CH₂)ₙAryl (often, phenyl or naphthyl, more often phenyl), (CH₂)ₙHeteroaryl;
R₆ is H, alkyl (preferably C₁-C₆ alkyl), C₁-C₆ alkoxy, (CH₂)ₙAryl (often, phenyl or naphthyl, more often phenyl), (CH₂)ₙHeteroaryl;
R₇ is H, alkyl (preferably C₁-C₆ alkyl), (CH₂)ₙAryl (often, phenyl or naphthyl, more often phenyl), (CH₂)ₙC(O)C₀-C₆ alkyl, —(CH₂)ₙR^{N1}N—C(O)—NR^{N2}R^{N3}, (CH₂)ₙ—S(O)₂Aryl, —OC(O)NR^{N1}R^{N2};
R_{8 is} H, OH, Halo, Nitro, C₁-C₆ hydroxyalkyl, (CH₂)ₙNR^{N1}R^{N2}, —(CH₂)ₙ—NR^{N1}—(CH₂)ₙ-Aryl (often, phenyl or naphthyl, more often phenyl), —NR^{N1}SO₂Aryl (often, phenyl or naphthyl, more often phenyl), (CH₂)ₙC₃-C₈cycloalkyl-NR^{N1}R^{N2}, C₁-C₆ alkoxy, O(CH₂)ₙaryl, (CH₂)ₙAryl (often, phenyl or naphthyl, more often phenyl), (CH₂)ₙHeteroaryl, C₁-C₆ alkyl, C₂-C₆ vinyl, C₂-C₆ alkynyl, —SO₂NR^{N1}R^{N2}, —OC(O)NR^{N1}R^{N2}, CONR^{N1}R^{N2};
R₉, R₁₀ and R₁₁ are each independently H, OH, Halo, Nitro, C₁-C₆ hydroxyalkyl, (CH₂)ₙNR^{N1}R^{N2}, —(CH₂)ₙ-NR^{N1}—(CH₂)ₙ-Aryl (often, phenyl or naphthyl, more often phenyl), —NR^{N1}SO₂Aryl (often, phenyl or naphthyl, more often phenyl), (CH₂)ₙC₃-C₈cycloalkyl-NR^{N1}R^{N2}, C₁-C₆ alkoxy, O(CH₂)ₙaryl, (CH₂)ₙ-Aryl (often, phenyl or naphthyl, more often phenyl), (CH₂)ₙHeteroaryl, C₁-C₆ alkyl, C₂-C₆ vinyl, C₂-C₆ alkynyl, —SO₂NR^{N1}R^{N2}, —OC(O)NR^{N1}R^{N2}, (CH₂)ₙC(O)OC₀-C₆ alkyl, (CH₂)ₙOC(O)C₀-C₆ alkyl or CONR^{N1}R^{N2};
R₁₂ is H, OH, hydroxyalkyl (preferably C₁-C₆ hydroxyalkyl), an optionally substituted (CH₂)ₙAryl (often, phenyl, benzyl or naphthyl, more often benzyl or naphthyl, the Aryl group being optionally substituted with one or two Halo groups, preferably F, Cl or Br, a nitro, CN or a C₁-C₆, preferably a C₁-C₃ alkyl group, preferably R₁₂ is an optionally substituted benzyl group or naphthyl group), (CH₂)ₙC₃-C₈cycloalkyl,
(CH₂)ₙC(O)NR^{N1}Aryl,
(CH₂)ₙ—C(O)C₀-C₆ alkyl;
R₁₃ is O or S;
R^{N1}, R^{N2} and R^{N3} are each independently H or a C₁-C₆ alkyl group which is optionally substituted with one or two hydroxyl groups and up to three halo groups (preferably F);
n is 0-12, preferably 0-6, often 0, 1, 2 or 3, or
a pharmaceutically acceptable salt, stereoisomer, solvate or polymorph thereof.

2. A compound according to claim 1 wherein R₁₂ is a benzyl group or a naphthyl group, each of which is optionally substituted with a C₁-C₆ alkyl group, a nitro group, a cyano group or one or two halo groups (preferably F, Cl or Br).

3. A compound according to claim 1 wherein the compound is a chemical structure according to a compound (pharmacore) 1a-q, 2a-q, 3a,b, 4a-e, 5a-e or 6a-e of FIG. 1, wherein R₁ and R₂ are each independently H, halo (preferably F, Cl or Br) or methoxy and R₃ is a phenyl, benzyl or naphthyl group, each of which is optionally substituted with 1 or 2 halo groups (preferably F, Cl or Br), a nitro group, a CN group or a C₁-C₆ alkyl group, preferably a C₁-C₃ group, most often a methyl group.

4. A compound according to claim 1 as set forth in FIG. 1A.

5. A compound according to claim 1 as set forth in FIG. 1B.

6. A compound according to claim 1 which is a compound selected from the group of compounds 5a-u of FIG. 1B.

7. A compound according to claim 1 which is compound 1d, 1f, 1h, 1k, 1l, 1o, 2d, 2l, 2o, 5b or 6d of FIGS. 1A and 1B.

8. A compound according to claim 1 which is compound 2f, 2g, 2l, 4e, 5a, 5c, 5a, 5e, 5f or 6b of FIGS. 1A and 1B.

9. A pharmaceutical composition comprising an effective amount of a compound according to claim 1, further in combination with a pharmaceutically acceptable carrier, additive or excipient.

10. The composition according to claim 9 further in combination with at least one additional bioactive agent.

11. The composition according to claim 10 wherein said bioactive agent is an additional anticancer agent.

12. The composition according to claim 11 wherein said additional cancer agent is iseverolimus, trabectedin, abraxane, TLK 286, AV-299, DN-101 , pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulator, a Bcl-2 inhibitor, an HDAC inhbitor, a c-MET inhibitor, a PARP inhibitor, a Cdk inhibitor, an EGFR TK inhibitor, an IGFR-TK inhibitor, an anti-HGF antibody, a PI3 kinase inhibitors, an AKT inhibitor, a JAK/STAT inhibitor, a checkpoint-1 or 2 inhibitor, a focal adhesion kinase inhibitor, a Map kinase kinase (mek) inhibitor, a VEGF trap antibody, pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111 , 131-I-TM-601, ALT-110, BIO 140, CC 8490, cilengitide, gimatecan, IL13-PE38QQR, INO 1001, IPdR₁ KRX-0402, lucanthone, LY 317615, neuradiab, vitespan, Rta 744, Sdx 102, talampanel, atrasentan, Xr 311, romidepsin, ADS-100380, sunitinib, 5-fluorouracil, vorinostat, etoposide, gemcitabine, doxorubicin, irinotecan, liposomal doxorubicin, 5′-deoxy-5-fluorouridine, vincristine, temozolomide, ZK-304709, seliciclib; PD0325901 , AZD-6244, capecitabine, L-Glutamic acid, N-[4-[2-(2-amino-4,7-dihydro-4-oxo-1 H-pyrrolo[2,3-d]pyrimidin-5-yl)ethyl]benzoyl]-, disodium salt, heptahydrate, camptothecin, PEG-labeled irinotecan, tamoxifen, toremifene citrate, anastrazole, exemestane, letrozole, DES(diethylstilbestrol), estradiol, estrogen, conjugated estrogen, bevacizumab, IMC-1C11, CHIR-258,); 3-[5-(methylsulfonylpiperadinemethyl)-indolylj-quinolone, vatalanib, AG-013736, AVE-0005, the acetate salt of [D-Ser(Bu t) 6, Azgly 10] (pyro-Glu-His-Trp-Ser-Tyr-D-Ser(Bu t)-Leu-Arg-Pro-Azgly-NH₂ acetate [C₅₉H₈₄N₁₈Oi₄-(C₂H₄O₂)ₓ where x=1 to 2.4], goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, Ionafarnib, BMS-214662, tipifarnib; amifostine, NVP-LAQ824, suberoyl analide hydroxamic acid, valproic acid, trichostatin A, FK-228, SU11248, sorafenib, KRN951, aminoglutethimide, arnsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gemcitabine, gleevac, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, irinotecan, topotecan, doxorubicin, docetaxel, vinorelbine, bevacizumab (monoclonal antibody) and erbitux, cremophor-free paclitaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, PTK787/ZK 222584, VX-745, PD 184352, rapamycin, 40-O-(2-hydroxyethyl)-rapamycin, temsirolimus, AP-23573, RAD001 , ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa, darbepoetin alfa, ipilumumab, vemurafenib among others among others, including immunotherapy agents such as IDO inhibitors (an inhibitor of indoleamine 2,3-dioxygenase (IDO) pathway) such as Indoximod (NLG-8187), Navoximod (GDC-0919) and NLG802, PDL1 inhibitors (an inhibitor of programmed death-ligand 1) including, for example, nivolumab, durvalumab and atezolizumab, PD1 inhibitors such as pembrolizumab (Merck) and CTLA-4 inhibitors (an inhibitor of cytotoxic T-lymphocyte associated protein 4/cluster of differentiation 152), including ipilimumab and tremelimumab, or a mixture thereof.

13. A method of modulating a sphingosine-1-phosphate (S1P) receptor comprising exposing said receptor to a compound according to claim 1.

14. (canceled)

15. (canceled)

16. (canceled)

17. A method of modulating a sphingosine-1-phosphate (S1P) receptor in a subject comprising administering to said subject an effective amount of a compound according to claim 1.

18. (canceled)

19. (canceled)

20. (canceled)

21. A method of treating a disease state or condition which is mediated through a sphingosine-1-phosphate (S1P) receptor in a subject in need comprising administering to said subject an effective amount of a compound according to claim 1.

22. A method of treating a disease state or condition selected from the group consisting of cancer, diabetes, inflammation, neurodegeneration (Alzheimer's disease, Parkinson's disease, Huntington's disease), multiple sclerosis, autoimmune disease, cardiovascular disease, including ischemia/reperfusion injury and stroke, schizophrenia, psoriasis, ulcerative colitis and inflammatory bowel syndrome in a subject in need comprising administering to said subject an effective amount of a compound according to claim 1.

23. The method according to claim 22 wherein said disease state or condition is cancer.

24. The method according to claim 23 wherein said cancer is selected from the group consisting of carcinomas (e.g., squamous-cell carcinomas, adenocarcinomas, hepatocellular carcinomas, and renal cell carcinomas), including those of the bladder, bone, bowel, breast, cervix, colon (colorectal), esophagus, head, kidney, liver, lung, nasopharyngeal, neck, ovary, pancreas, prostate, and stomach; leukemias, including acute myelogenous leukemia, acute lymphocytic leukemia, acute promyelocytic leukemia (APL), acute T-cell lymphoblastic leukemia, adult T-cell leukemia, basophilic leukemia, eosinophilic leukemia, granulocytic leukemia, hairy cell leukemia, leukopenic leukemia, lymphatic leukemia, lymphoblastic leukemia, lymphocytic leukemia, megakaryocytic leukemia, micromyeloblastic leukemia, monocytic leukemia, neutrophilic leukemia and stem cell leukemia; benign and malignant lymphomas, including Burkitt's lymphoma, Non-Hodgkin's lymphoma and B-cell lymphoma; benign and malignant melanomas; myeloproliferative diseases; sarcomas, including Ewing's sarcoma, hemangiosarcoma, Kaposi's sarcoma, liposarcoma, myosarcomas, peripheral neuroepithelioma, and synovial sarcoma; tumors of the central nervous system (e.g., gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas); germ-line tumors (e.g., bowel cancer, breast cancer, prostate cancer, cervical cancer, uterine cancer, lung cancer (e.g., small cell lung cancer, mixed small cell and non-small cell cancer, pleural mesothelioma, including metastatic pleural mesothelioma small cell lung cancer and non-small cell lung cancer), ovarian cancer, testicular cancer, thyroid cancer, astrocytoma, esophageal cancer, pancreatic cancer, stomach cancer, liver cancer, colon cancer, and melanoma); mixed types of neoplasias, particularly carcinosarcoma and Hodgkin's disease; and tumors of mixed origin, such as Wilms' tumor and teratocarcinomas.

25. The method according to claim 23 wherein said cancer is selected from the group consisting of choriocarcinoma, testicular choriocarcinoma, non-seminomatous germ cell testicular cancer, placental cancer (trophoblastic tumor) and embryonal cancer.

26. (canceled)

27. The method according to claim 22 wherein said autoimmune disease is rheumatoid arthritis, antiphospholipid antibody syndrome, lupus, chronic urticaria, Sjogren's disease, autoimmune-related Type 1 diabetes, rheumatoid arthritis (RA), psoriasis/psoriatic arthritis, multiple sclerosis, inflammatory bowel disease (IBD) including Crohn's disease and ulcerative colitis, Addison's disease, Grave's disease, Hashimoto's thyroiditis, Myasthenia gravis, autoimmune vasculitis, pernicious anemia and celiac disease.

28-42. (canceled)
